(19) United States
(12) Patent Application Publication
(10) Pub. No.: US 2026/0231675 A1
(43) Pub. Date: Aug. 6, 2026

(54) ORGANIC COMPOUND AND ORGANIC ELECTROLUMINESCENT DEVICE USING SAME

(71) Applicant: SOLUS ADVANCED MATERIALS CO., LTD., Iksan-si, Jeollabuk-do (KR)

(72) Inventors: Areum CHO, Seongnam-si (KR); Sukwon CHO, Seongnam-si (KR); Jaeyi SIM, Seongnam-si (KR); Hyungchan BAE, Seongnam-si (KR)

(73) Assignee: SOLUS ADVANCED MATERIALS CO., LTD., Iksan-si, Jeollabuk-do (KR)

(21) Appl. No.: 19/141,539

(22) PCT Filed: Dec. 21, 2023
(86) PCT No.: PCT/KR2023/021220

§ 371 Date: Jun. 20, 2025

(30) Foreign Application Priority Data
Dec. 23, 2022 (KR) 10-2022-0183802

(51) Int. Cl. H10K 85/60; C07D 491/107; C07D 495/10; H10K 50/11; H10K 50/16

(52) U.S. Cl. CPC H10K 85/654; C07D 491/107; C07D 495/10; H10K 85/615; H10K 85/6572; H10K 85/6574; H10K 85/6576; H10K 50/11; H10K 50/16

(57) ABSTRACT

The present invention relates to: a novel compound having excellent carrier transport capacity, luminescence capacity, and thermal stability; and an organic electroluminescent device of which characteristics such as luminescence efficiency, drive voltage, and lifetime are improved due to the inclusion of the novel compound in one or more organic layers.

### TECHNICAL FIELD

The present invention relates to a novel organic compound and an organic electroluminescent device using the same and, more specifically, to a novel compound with excellent electron transporting ability, and an organic electroluminescent device exhibiting improved characteristics, such as luminous efficiency, driving voltages, and lifespan, by containing the novel compound in one or more organic layers.

### BACKGROUND ART

In an organic electroluminescent device, upon the application of voltage between two electrodes, holes from an anode and electrons from a cathode are injected into organic layers. The injected holes and electrons combine with each other to form excitons, and the excitons fall down to the ground state to emit light. Particularly, materials used for the organic layers may be classified into light emission materials, hole injection materials, hole transport materials, electron transport materials, electron injection materials, and the like according to the function thereof.

Materials for forming an emissive layer of the organic electroluminescent device may be classified into blue, green and red light emission materials according to the color of light emission. Additionally, yellow and orange light emission materials may be used as light emission materials for displaying better natural colors. Additionally, host/dopant-based light emission materials may be used as light emission materials to increase color purity and improve luminous efficiency through energy transfer.

Dopant materials may be classified into fluorescent dopants using organic materials and phosphorescent dopants using metal complex compounds containing heavy atoms, such as Ir and Pt. These phosphorescent materials can theoretically improve the luminous efficiency up to four times compared to fluorescent materials, so research has been conducted on phosphorescent host materials as well as phosphorescent dopants.

Until today, NPB, BCP, Alq₃, and the like have been widely known as materials for use in a hole injection layer, a hole transport layer, and an electron transport layer, and anthracene derivatives have been reported as the light emitting layer materials. In particular, among the light emitting layer materials, metal complex compounds containing Ir, such as Firpic, Ir(ppy)₃, (acac)Ir(btp)₂, and the like, which have advantages in terms of improving efficiency, are used as blue, green, and red phosphorescent dopant material, and 4,4-dicarbazollybiphenyl (CBP) is used as the phosphorescent host material.

However, conventional light emission materials have advantages in terms of light emission characteristics, but are not satisfactory in terms of lifespan of organic electroluminescent devices due to low glass transition temperatures and very poor thermal stability. Accordingly, there is a need to develop light emission materials with excellent performance.

### DISCLOSURE OF INVENTION

### Technical Problem

The present invention has been made to solve the above-mentioned problems, and an aspect of the present invention is to provide a novel compound, which can be used as a material for an organic layer of an organic electroluminescent device, specifically, a material for an electron transport layer, an auxiliary electron transport layer, or an emissive layer, due to excellent heat resistance, carrier transporting ability, and light emitting ability.

Another aspect of the present invention is to provide an organic electroluminescent device exhibiting a low driving voltage, high luminous efficiency, and improved lifespan by containing the above-described novel compound.

Other purposes and advantages of the present invention will be clarified by following detailed description and claims.

### Solution to Problem

To achieve the above objectives, the present invention provides a compound represented by the following Chemical Formula 1:

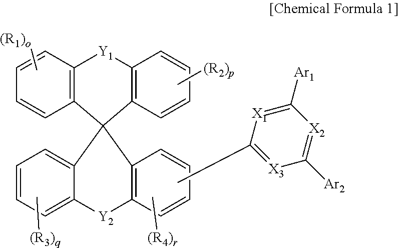

wherein in Chemical Formula 1,
X₁ to X₃ are the same as or different from each other and are each independently C(R₅) or N, provided that at least two of X₁ to X₃ are N;
Y₁ and Y₂ are the same as or different from each other and are each independently selected from the group consisting of O, S, and NR₁₁, provided that the case where both Y₁ and Y₂ are O is excluded;
Ar₁ and Ar₂ are the same as or different from each other and are each independently selected from the group consisting of a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 ring atoms, a C₆-C₆₀ aryl group, a heteroaryl group having 5 to 60 ring atoms, a C₁-C₄₀ alkyloxy group, a C₆-C₆₀ aryloxy group, a C₃-C₄₀ alkylsilyl group, a C₆-C₆₀ arylsilyl group, a C₁-C₄₀ alkylboron group, a C₆-C₆₀ arylboron group, a C₆-C₆₀ arylphosphine group, a C₆-C₆₀ arylphosphine oxide group, and a C₆-C₆₀ arylamine group, or combine with an adjacent group to form a fused ring;
R₁ to R₅ and R₁₁ are the same as or different from one another and are each independently selected from the group consisting of a hydrogen, a deuterium (D), a halogen, a cyano group, a nitro group, a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 ring atoms, a C₆-C₆₀ aryl group, a heteroaryl group having 5 to 60 ring atoms, a C₁-C₄₀ alkyloxy group, a C₆-C₆₀ aryloxy group, a C₃-C₄₀ alkylsilyl group, a C₆-C₆₀ arylsilyl group, a C₁-C₄₀ alkylboron group, a C₆-C₆₀ arylboron group, a C₆-C₆₀ aryl phosphine group, a C₆-C₆₀ arylphosphine oxide group, and a C₆-C₆₀ arylamine group, or combine with an adjacent group to form a fused ring;
o, p, and q are each independently an integer of 0 to 4, and r is an integer of 0 to 3; and
the alkyl group, alkenyl group, alkynyl group, aryl group, heteroaryl group, aryloxy group, alkyloxy group, cycloalkyl group, heterocycloalkyl group, alkylsilyl group, arylsilyl group, alkylboron group, arylboron group, arylphosphine group, arylphosphine oxide group, and arylamine group of Ar₁ to Ar₂, R₁ to R₅, and R₁₁ may be each independently substituted with at least one substituent selected from the group consisting of a deuterium (D), a halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 ring atoms, a C₆ to C₆₀ aryl group, a heteroaryl group having 5 to 60 ring atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, and wherein when the substituent is present in a plural number, they may be the same or different from each other.

In addition, the present invention also provides an organic electroluminescent device including: an anode, a cathode, and one or more organic layers disposed between the anode and the cathode, wherein at least one of the one or more organic layers includes the compound represented by Chemical Formula 1.

In such a case, the organic layer including the compound represented by Chemical Formula 1 may be selected from a light emitting layer, a light emitting auxiliary layer, a hole injection layer, a hole transport layer, an electron injection layer, a lifespan improvement layer, an electron transport layer, and an auxiliary electron transport layer. In such a case, the compound represented by Chemical Formula 1 may be included as a material for at least one of a phosphorescent host material of a light emitting layer, an electron transport layer and an auxiliary electron transport layer.

### Effects of the Invention

According to an embodiment of the present invention, a compound represented by Chemical Formula 1 has excellent characteristics such as an electron transport ability, luminescence ability, heat resistance, and the like, and thereby is applicable as an organic layer material of an organic electroluminescent device.

In particular, when the compound represented by Chemical Formula 1 of the present invention is used as an electron transport layer material or an auxiliary electron transport layer material, it may exhibit high thermal stability, low driving voltage, rapid mobility, high current efficiency and long lifespan characteristics compared to conventional host materials or electron transport materials.

Accordingly, the organic electroluminescent device including the compound of Chemical Formula 1 may be significantly improved in aspects such as excellent light emitting performance, low driving voltage, long lifespan, and high efficiency, and thus may be effectively applied to full color display panels and the like.

Effects according to the present invention are not limited by the description exemplified above, and more diverse effects are included in the present specification.

### DETAILED DESCRIPTION OF THE INVENTION

Hereinafter, the present invention will be described in detail.

### <Novel Organic Compound>

The present invention provides a novel compound having excellent thermal stability, carrier transport capability, and luminescence efficiency, for example, a polycyclic spiro-based compound.

The compound represented by Chemical Formula 1 has a polycyclic spiro binding structure including at least two hetero atoms (e.g., S—S/O—S/S—NR′/NR′—NR″/O—NR′), and an electron withdrawing group (EWG) having excellent electron transport ability is directly bonded to the core structure to form a basic skeleton.

The compound represented by Chemical Formula 1 has a core with a polycyclic spiro-linked structure containing at least two heteroatoms (e.g., S—S/O—S/S—NR′/NR′—NR″/O—NR′), and an electron withdrawing group (EWG) with excellent electron transport ability is directly bonded to the core structure to form a basic skeleton the compound.

Specifically, the compound of Chemical Formula 1 exhibits an electron-donating effect due to the lone pair electrons of the heteroatom (O or S) included in the polycyclic spiro-based core structure. Therefore, when such the compound is applied to an organic light-emitting device, the luminous efficiency of the device can be improved, and the durability and stability of the device are enhanced, thereby effectively increasing the lifespan of the device.

Furthermore, the introduction of triazine or pyrimidine moiety, which is an azine-based functional group having a strong electron withdrawing group (EWG), for the improvement of electron mobility leads to physicochemical properties that are more suitable for electron injection or electron transport. The application of such a compound of Chemical Formula 1 as a material for an electron transport layer or auxiliary electron transport layer enables the layers to favorably receive electrons from a cathode and thus smoothly transport the electrons to a light emitting layer, thereby lowering the driving voltage of the device and inducing high efficiency and long lifespan of the device.

Particularly, when an electron withdrawing group (EWG) (e.g., an azine group) is substituted on a nitrogen-containing ring in the polycyclic spiro-based core structure, the EWG effect can be further enhanced due to the influence of N atom with lower electronegativity than O, thereby lowering the driving voltage of the device and inducing high efficiency of the device. In addition, when an EWG is substituted on a ring other than a N- or S-containing ring in the polycyclic spiro-based core structure, such a structure can relatively lower the packing density during the manufacturing of the device since NR¹¹ has a bulkier structure than an oxygen atom and S atom also possesses a larger orbital region than O atom. The above-described effects lead to a reduction in mobility, thereby configuring a stable device with long lifespan.

Additionally, the compound represented by Chemical Formula 1 of the present invention has steric hindrance due to the spiro-type structure, and thus can prevent crystallization and maintain high thermal stability during the formation of films, thereby exhibiting stability at a high deposition temperature. In addition, the spiro-type core structure has superior electrochemical stability, a high glass transition temperature (Tg), and excellent carrier transport capability. Therefore, an increase in efficiency, a low driving voltage, and an improvement in lifespan characteristics can be achieved in the organic electroluminescent device.

As described above, the compound represented by Chemical Formula 1 of the present invention may be applicable as an organic layer material of an organic electroluminescent device, preferably a light-emitting layer material (a blue, a green and/or a red phosphorescent host material), an electron transport/injection layer material, a hole transport/injection layer material, a light-emitting auxiliary layer material, an auxiliary electron transport layer, and/or a lifespan enhancement layer material. In particular, when the compound of the present invention is used as a material for an electron transport layer or an electron transport auxiliary layer, excellent improvements in device efficiency, driving voltage, and lifespan characteristics can be expected. In addition, the organic electroluminescent device including the compound of the above chemical formula may be greatly improved in terms of the performance and lifespan characteristics, and the performance a full color organic electroluminescent panel to which the organic electroluminescent device is applied may also be maximized.

According to the present invention, the compound represented by Chemical Formula 1 has a polycyclic spirobixanthene group including at least two heteroatoms (e.g., S—S/O—S/S—NR′/NR′—NR″/O—NR′) as a core structure, and a nitrogen-containing heteroaromatic ring (e.g., an azine, a X₁ to X₃-containing ring) having excellent electron withdrawing group-(EWG) properties with excellent electron transport capability is directly bonded to the core structure to form a basic skeleton structure.

The polycyclic spiro-core structure includes at least two heteroatoms (e.g., a Y₁ and Y₂-containing ring). As one example of the polycyclic spiro core structure, Y₁ and Y₂ may be the same as or different from each other, and may each independently be selected from the group consisting of O, S and NR₁₁, provided that both Y₁ and Y₂ are not O. Specifically, Y₁ and Y₂ are preferably different from each other.

Herein, R₁₁ may each independently be selected from the group consisting of a hydrogen, a deuterium (D), a halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, or combine with an adjacent group to form a fused ring. In this case, when R₁₁ may be plural in number, the plurality of R₁₁ may be the same as or different from each other. Specifically, R₁₁ may preferably be selected from: a hydrogen, a deuterium (D), a C₁ to C₄₀ alkyl group, a C₆ to C₆₀ aryl group, and a heteroaryl group having 5 to 60 nuclear atoms.

In an embodiment of the present invention, the polycyclic spiro-core structure (e.g., Y₁~Y₂-containing ring) may be more specifically embodied as any one of Chemical Formulas 2 to Chemical Formula 5 below. However, it is not limited thereto.

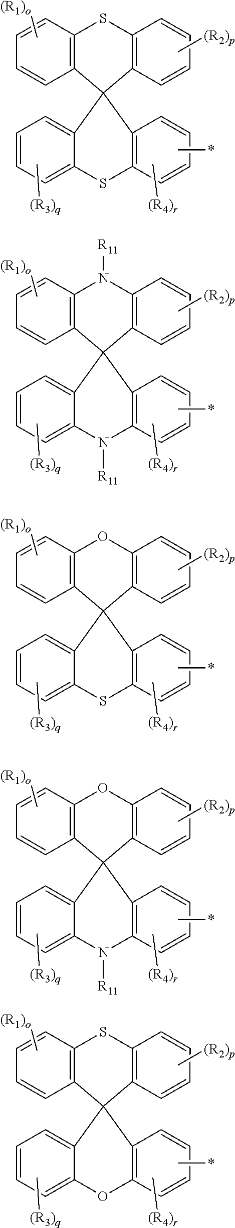

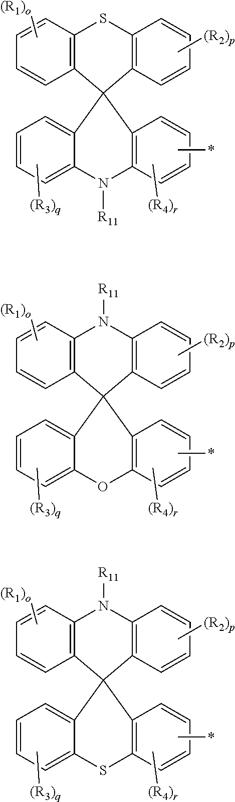

wherein the formulas,
* may indicate a site where a bond with Chemical Formula is made,
R₁₁ may be selected from the group consisting of a hydrogen, a deuterium (D), a C₁ to C₄₀ alkyl group, a C₆ to C₃₀ aryl group, and a heteroaryl group having 5 to 30 nuclear atoms,
R₁ to R₄, o, p, q and r are each as defined in Chemical Formula 1.
the polycyclic spiro-core structure (e.g., Y₁~Y₂-containing ring) according to the present invention may each be substituted with R₁ to R₄ as various substituents. Such R₁ to R₄ may be the same as or different from each other, and may each independently be selected from the group consisting of a hydrogen, a deuterium (D), a halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, or combine with an adjacent group to form a fused ring. In this case, when R₁ to R₄ may be plural in number, the plurality of R₁ to R₄ may be the same as or different from each other. Specifically, R₁ to R₄ may be the same as or different from each other, and may preferably be selected from: a hydrogen, a deuterium (D), a cyano group, a C₁ to C₄₀ alkyl group, a C₆ to C₆₀ aryl group, and a heteroaryl group having 5 to 60 nuclear atoms, or combine with adjacent group to form a fused ring.

Herein, o, p, and q may each independently be an integer of 0 to 4, and r may be an integer of 0 to 3. When o is 0, R₁ may be a hydrogen, and when o is an integer of 1 to 3, R₁ may have the aforementioned substituents other than hydrogen. p, q, and r may also be equally applied.

In Chemical Formula 1 according to the present invention, a nitrogen-containing heteroaromatic ring (e.g., an azine, X₁ to X₃-containing ring) having an EWG property with excellent electron transport ability is directly bonded to one side phenyl ring of the polycyclic spiro-co0re structure (e.g., Y₁~Y₂ containing ring).

the nitrogen-containing heterocycle (e.g., X₁ to X₃-containing ring) may be a monocyclic or polycyclic heteroaryl group (e.g., azine) containing at least two nitrogen atoms. For an example of the nitrogen-containing heteroaromatic ring (e.g., X₁ to X₃-containing ring), X₁ to X₃ may be the same as or different from each other, and may each independently be C(R₅) or N, provided that at least two of X₁ to X₃ are N. For a specific example, X₁ to X₃ includes two to three nitrogen atoms (N). As such, since the heterocycle containing two to three nitrogen atoms (N) is included, more excellent electron absorption characteristics may be exhibited, which is advantageous for electron injection and transport.

Herein, R₅ may be selected from the group consisting of a hydrogen, a deuterium (D), a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ monoarylphosphinyl group, a C₆ to C₆₀ diarylphosphinyl group, a C₆ to C₆₀ arylamine group, a C₅ to C₆₀ arylheteroarylamine group, and a heteroarylamine group having 5 to 60 nuclear atoms, or combine with an adjacent group to form a fused ring. In this case, when R₅ may be plural in number, the plurality of R₅ may be the same as or different from each other. Specifically, R₁₁ may preferably be selected from: a hydrogen, a deuterium (D), a C₁ to C₄₀ alkyl group, a C₆ to C₆₀ aryl group, and a heteroaryl group having 5 to 60 nuclear atoms.

In an embodiment of the present invention, the nitrogen-containing heterocycle (e.g., X₁ to X₃-containing ring) may be more specifically embodied as any one of the following structural formulas. However, it is not limited thereto.

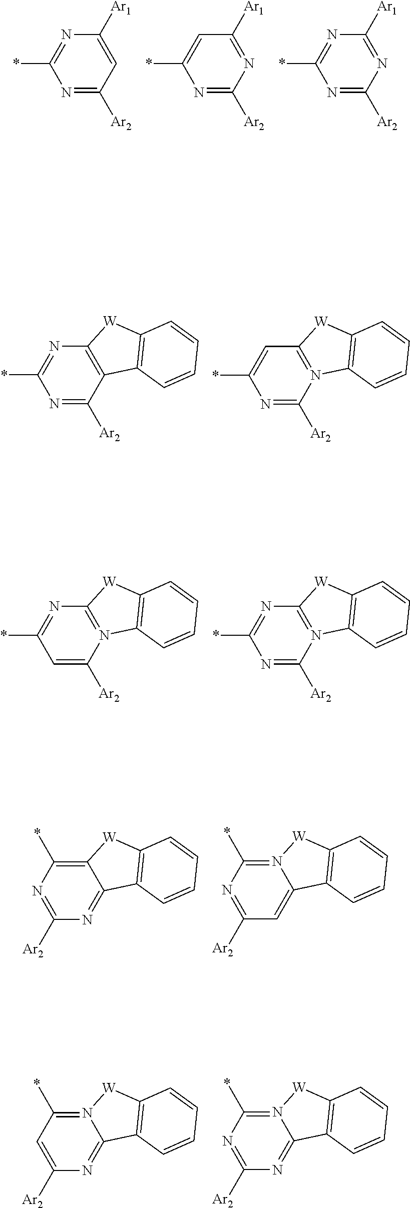

wherein the formulas,
* indicates a site where a bond with Chemical Formula 1 is made,
W is O or S, and
Ar₁ and Ar₂ are each as defined in Chemical Formula 1.
the nitrogen-containing heterocycle (e.g., X₁ to X₃-containing ring) according to the present invention may each be substituted with Ar₁ to Ar₂ as various substituents. Such Ar₁ and Ar₂ may be the same as or different from each other and may each independently be selected from: a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, or combine with an adjacent group to form a fused ring. specifically, Ar₁ and Ar₂ may be each independently selected from: a C₆ to C₁₈ aryl group, and a heteroaryl group having 5 to 18 nuclear atoms.

In an embodiment of the present invention, Ar₁ and Ar₂ may be the same as or different from each other and may each independently be more specifically embodied as any one of the following structural formulas. However, it is not limited thereto.

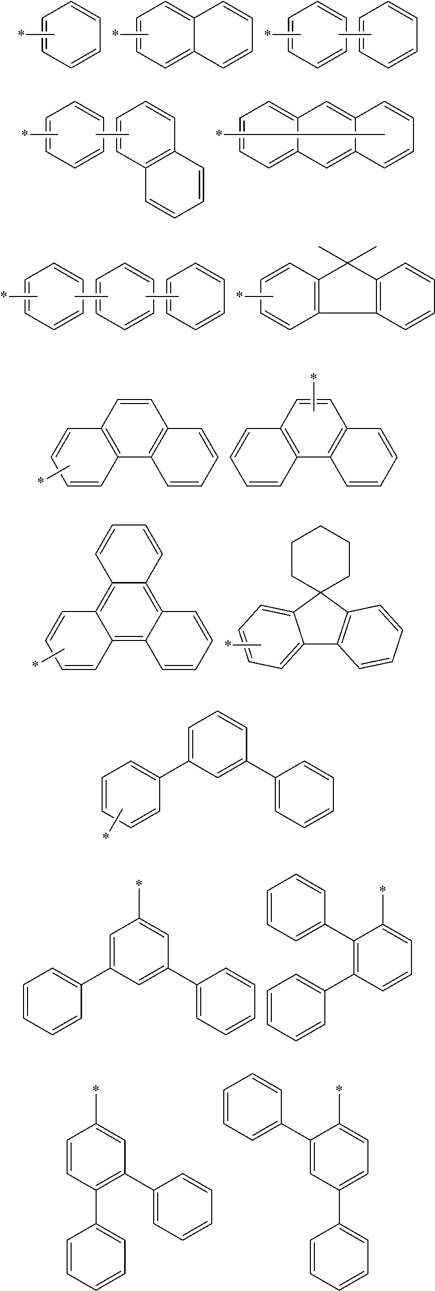

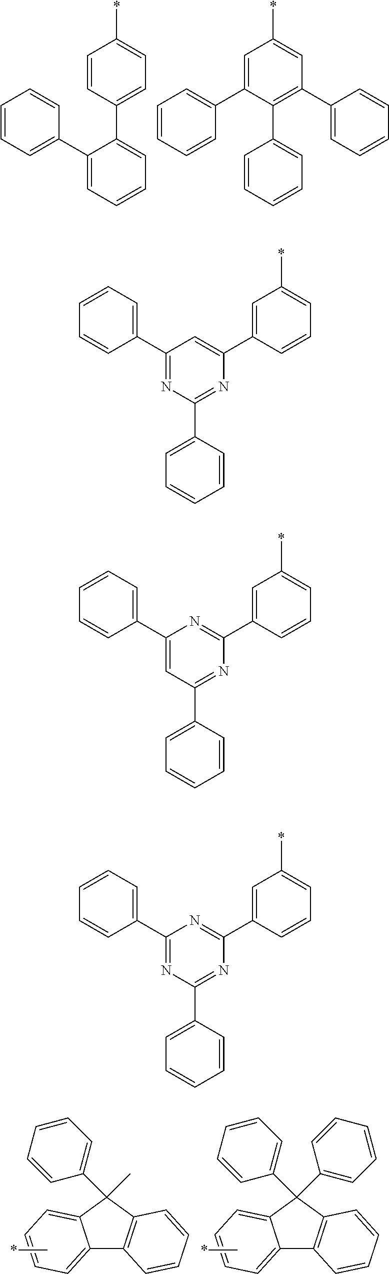

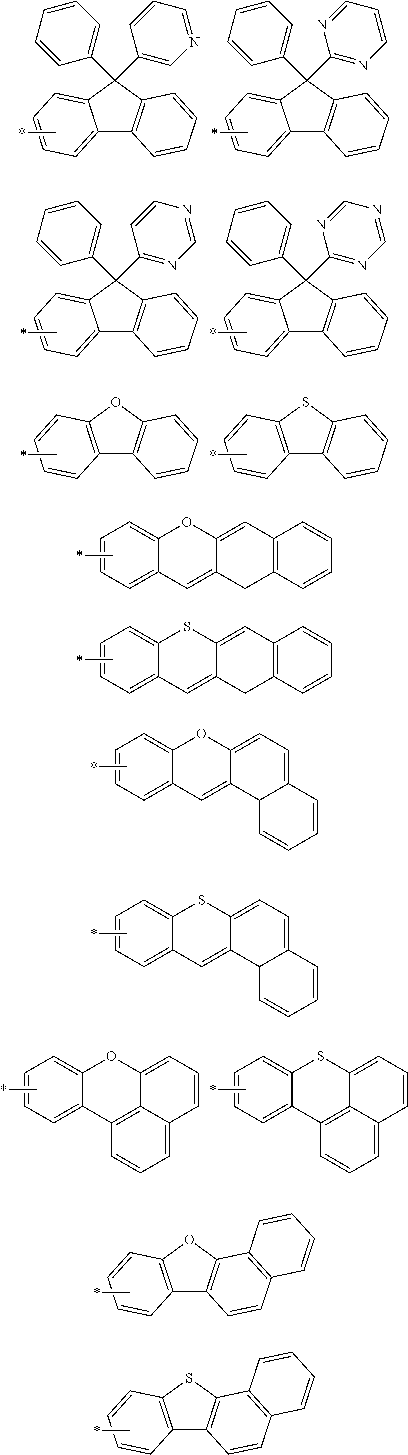

wherein the formulas,
* indicates a site where a bond with Chemical Formula 1 is made. In addition, although not shown in the above structural formulas, at least one substituent known in the art (e.g., the same as the definition of R₅) may be substituted.

In the above Chemical Formula 1, the alkyl, alkenyl, alkynyl, cycloalkyl, heterocycloalkyl, aryl, heteroaryl, alkyloxy, aryloxy, alkylsilyl, arylsilyl, alkylboron, arylboron, arylphosphine, arylphosphine oxide, and arylamine groups of Ar₁ to Ar₂, R₁ to R₅ and R₁₁ are optionally each independently unsubstituted or substituted with at least one selected from the group consisting of: a deuterium (D), a halogen, a cyano group, a nitro group, a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 nuclear atoms, a C₆-C₆₀ aryl group, a heteroaryl group having 5 to 60 nuclear atoms, a C₁-C₄₀ alkyloxy group, a C₆-C₆₀ aryloxy group, a C₁-C₄₀ alkylsilyl group, a C₆-C₆₀ arylsilyl group, a C₁-C₄₀ alkylboron group, a C₆-C₆₀ arylboron group, a C₁-C₄₀ arylphosphine group, a C₆-C₆₀ aryl phosphine oxide group, and a C₆-C₆₀ arylamine group, and provided that when the substituent is present in a plural number, they are optionally the same or different from each other.

In an embodiment of the present invention, the compound represented by Chemical Formula 1 may be more specifically represented by any one of the following Chemical Formulas 2 to 9 according to the type of the heteroatoms introduced into the polycyclic spiro core structure. However, it is not limited thereto.

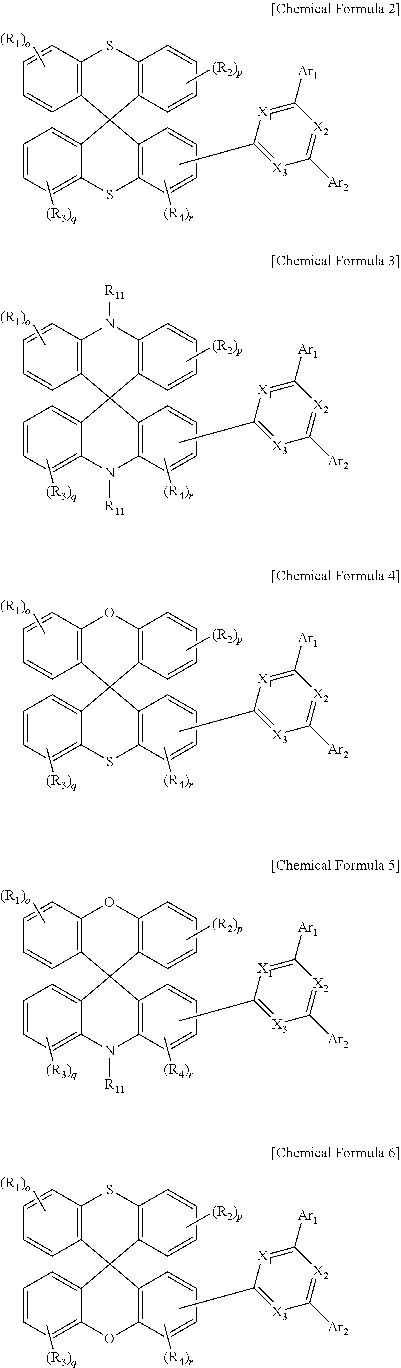

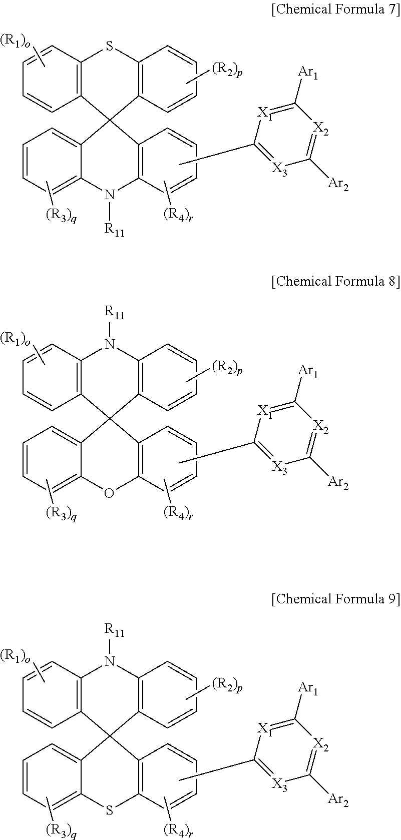

wherein the formulas,
X₁ to X₃, Ar₁ to Ar₂, R₁ to R₄, R₁₁, o, p, q and r are each as defined in Chemical Formula 1.

In another embodiment of the present invention, the compound represented by Chemical Formula 1 may be more specifically represented by any one of the following Chemical Formulas 10 to 20 according to the type of the nitrogen-containing heterocycle (e.g., X₁ to X₃-containing ring). However, it is not limited thereto.

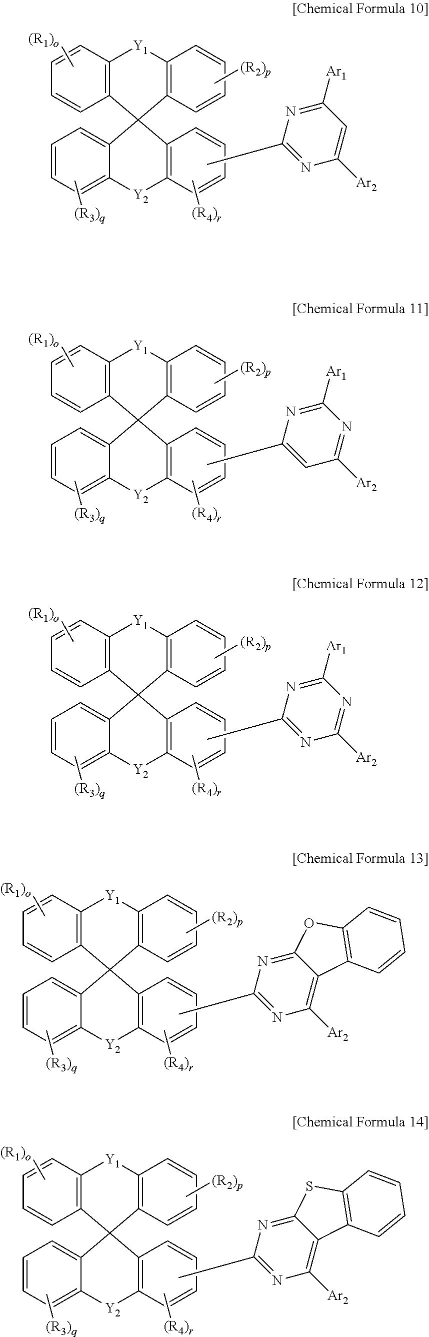

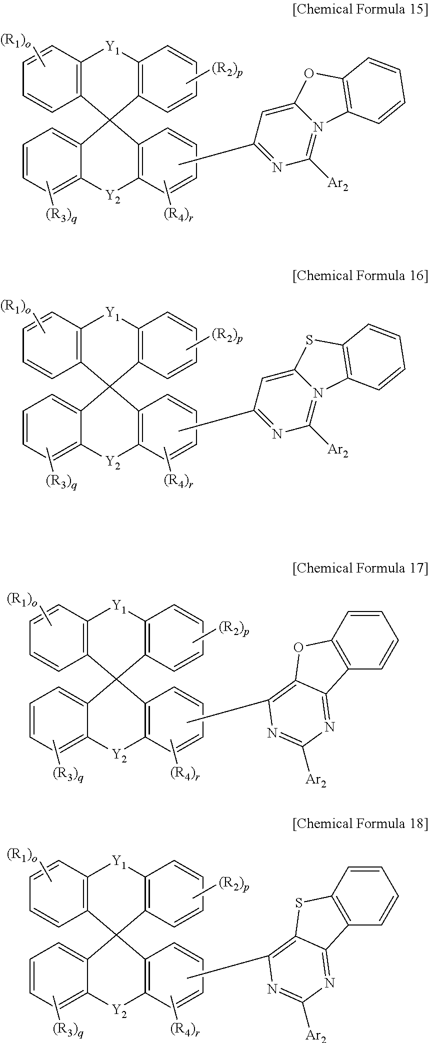

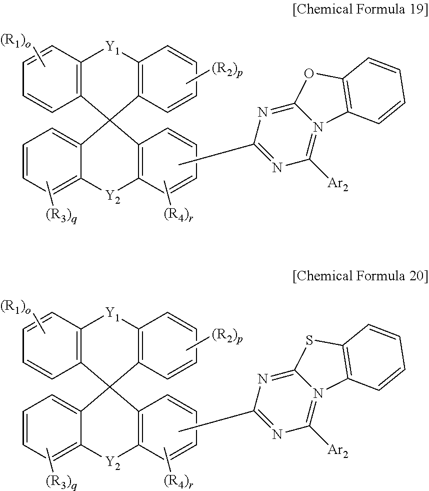

wherein the formulas,
W is O or S,
Y₁ to Y₂, Ar₁ to Ar₂, R₁ to R₄, o, p, q and r are the same as defined in Chemical Formula 1.

In another embodiment of the present invention, the compound represented by Chemical Formula 1 may be more specifically represented by any one of the following Chemical Formulas 21 to 28 according to the bonding position of the fused ring formed on the polycyclic spiro core. However, it is not limited thereto.

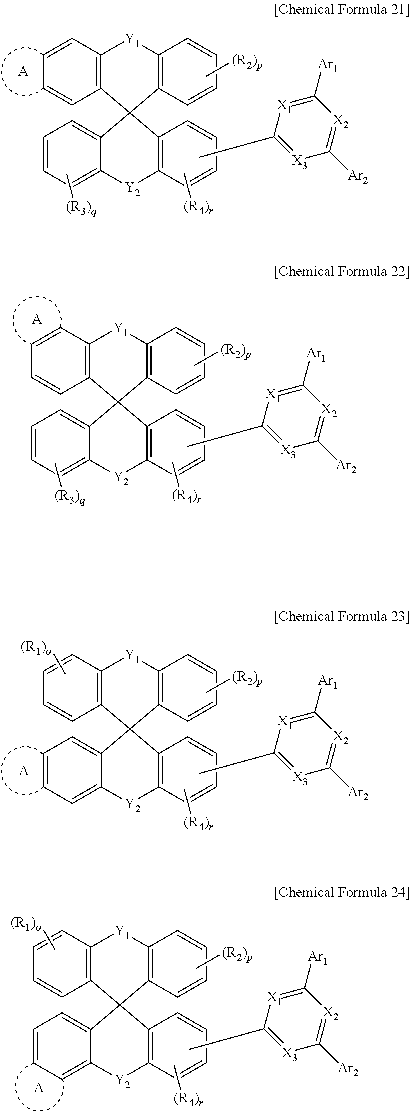

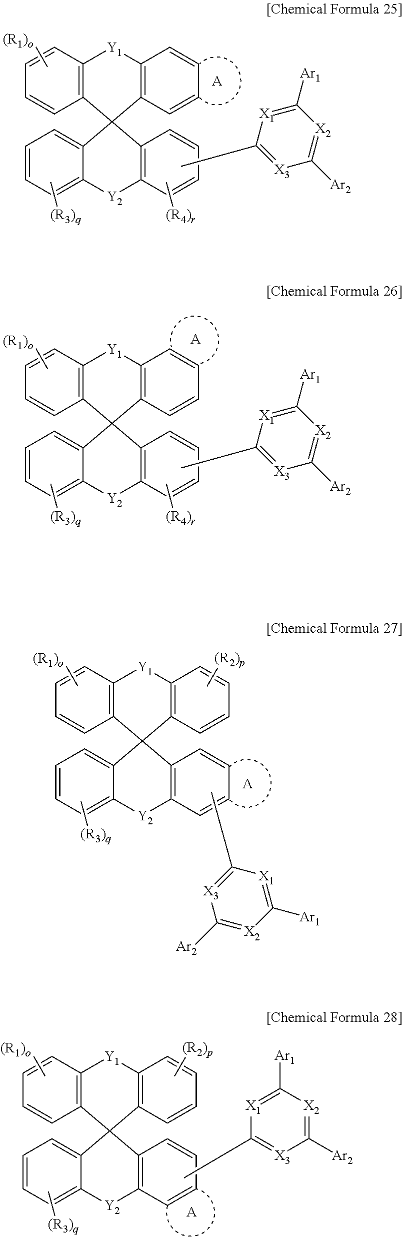

wherein the formulas,

Ring A may be a conventional hydrocarbon-based ring or a hydrocarbon-based ring containing at least one heteroatom known in the art, and may be in a form in which they are condensed, fused, bridged, or spirocyclic bonded to other rings (e.g., core structures) adjacent to each other. For example, the Ring A may be selected from the group consisting of: a monocyclic or polycyclic alicyclic ring, a monocyclic or polycyclic heteroalicyclic ring, a monocyclic or polycyclic aromatic ring, or a monocyclic or polycyclic heteroaromatic ring. Specifically, the Ring A may be preferably an aromatic ring of C₆ to C₁₈, or a heteroaromatic ring having 5 to 18 nuclear atoms.

X₁ to X₃, Y₁ to Y₂, Ar₁ to Ar₂, R₁ to R₄, o, p, q and r are each as defined in Chemical Formula 1.

In another embodiment of the present invention, the compound represented by Chemical Formula 1 may be more specifically represented by any one of the following Chemical Formulas 29 to 31 according to the bonding position of the nitrogen-containing heterocycle. However, it is not limited thereto.

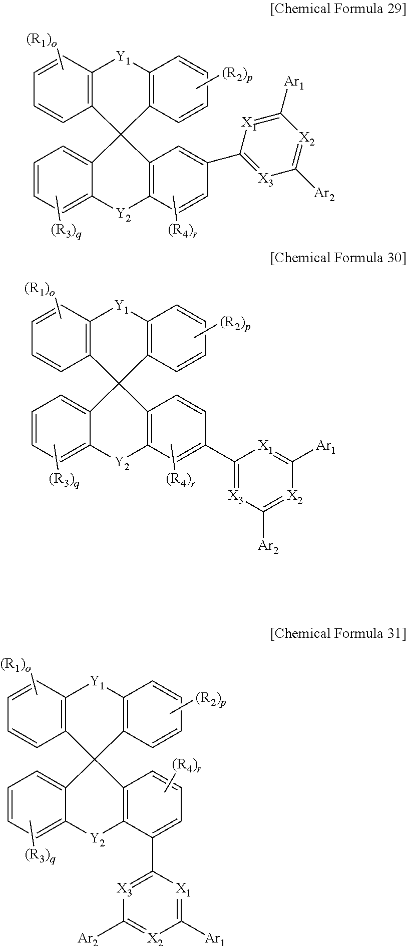

wherein the formulas,
X₁ to X₃, Y₁ to Y₂, Ar₁ to Ar₂, R₁ to R₄, o, p, q and r are the same as defined in Chemical Formula 1.

The compound represented by Chemical Formula 1 of the present invention described above may be further embodied as compounds represented by the following compounds represented by, for example, 1 to 120. However, the compound represented by Chemical Formula 1 of the present invention is not limited to those exemplified below.

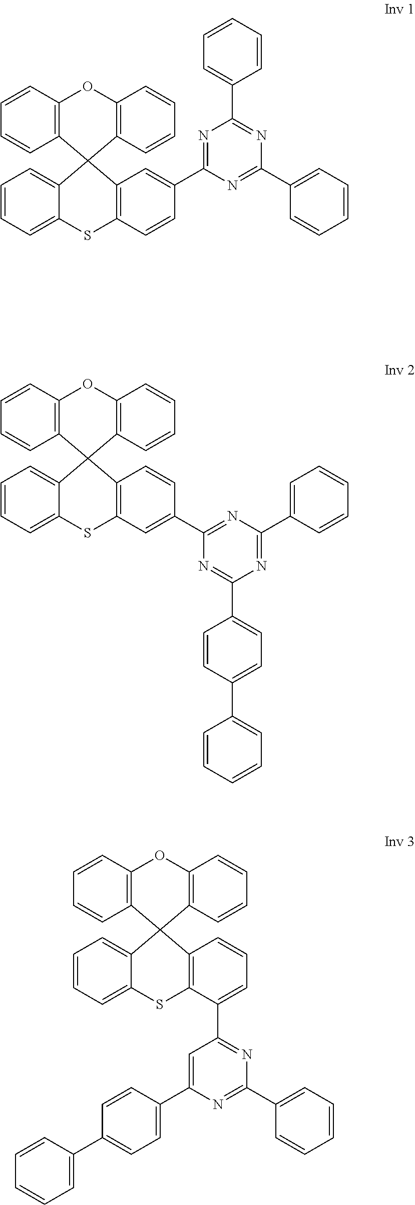 [SMILES: c1ccc(-c2nc(-c3ccccc3)nc(-c3ccc4c(c3)C3(c5ccccc5Oc5ccccc53)c3ccccc3S4)n2)cc1 ; c1ccc(-c2ccc(-c3nc(-c4ccccc4)nc(-c4ccc5c(c4)Sc4ccccc4C54c5ccccc5Oc5ccccc54)n3)cc2)cc1 ; c1ccc(-c2ccc(-c3cc(-c4cccc5c4Sc4ccccc4C54c5ccccc5Oc5ccccc54)nc(-c4ccccc4)n3)cc2)cc1]

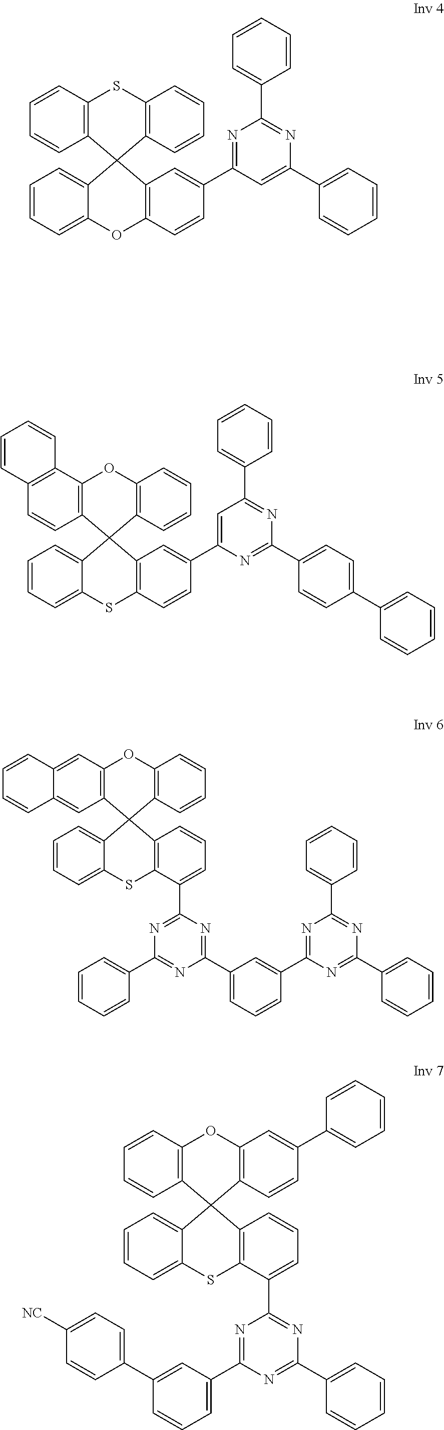 [SMILES: c1ccc(-c2cc(-c3ccc4c(c3)C3(c5ccccc5O4)c4ccccc4Sc4ccccc43)nc(-c3ccccc3)n2)cc1 ; c1ccc(-c2ccc(-c3nc(-c4ccccc4)cc(-c4ccc5c(c4)C4(c6ccccc6Oc6c4ccc4ccccc64)c4ccccc4S5)n3)cc2)cc1 ; c1ccc(-c2nc(-c3ccccc3)nc(-c3cccc(-c4nc(-c5ccccc5)nc(-c5cccc6c5Sc5ccccc5C65c6ccccc6Oc6cc7ccccc7cc65)n4)c3)n2)cc1 ; N#Cc1ccc(-c2cccc(-c3nc(-c4ccccc4)nc(-c4cccc5c4Sc4ccccc4C54c5ccccc5Oc5cc(-c6ccccc6)ccc54)n3)c2)cc1]

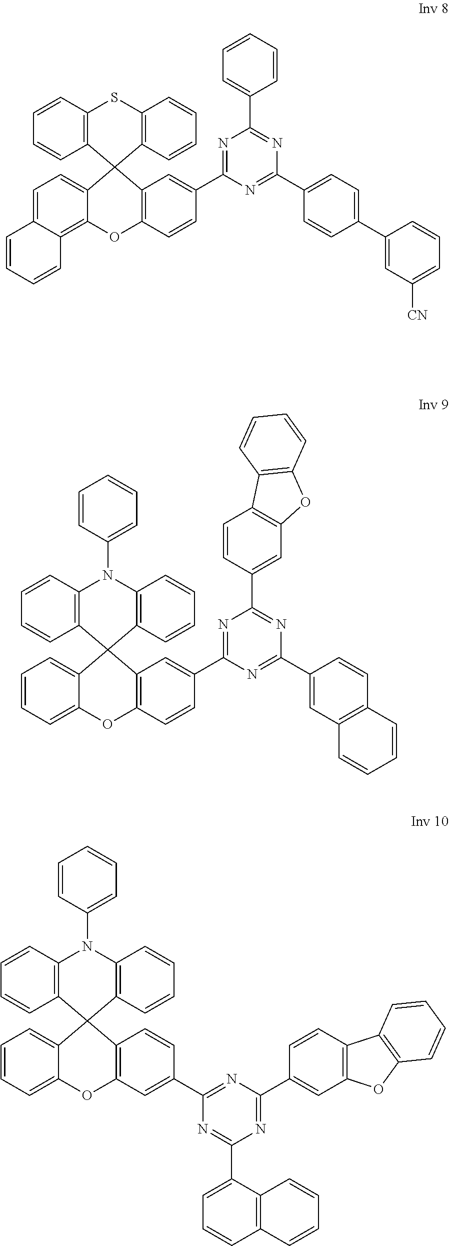 [SMILES: N#Cc1cccc(-c2ccc(-c3nc(-c4ccccc4)nc(-c4ccc5c(c4)C4(c6ccccc6Sc6ccccc64)c4ccc6ccccc6c4O5)n3)cc2)c1 ; c1ccc(N2c3ccccc3C3(c4ccccc4Oc4ccc(-c5nc(-c6ccc7ccccc7c6)nc(-c6ccc7c(c6)oc6ccccc67)n5)cc43)c3ccccc32)cc1 ; c1ccc(N2c3ccccc3C3(c4ccccc4Oc4cc(-c5nc(-c6ccc7c(c6)oc6ccccc67)nc(-c6cccc7ccccc67)n5)ccc43)c3ccccc32)cc1]

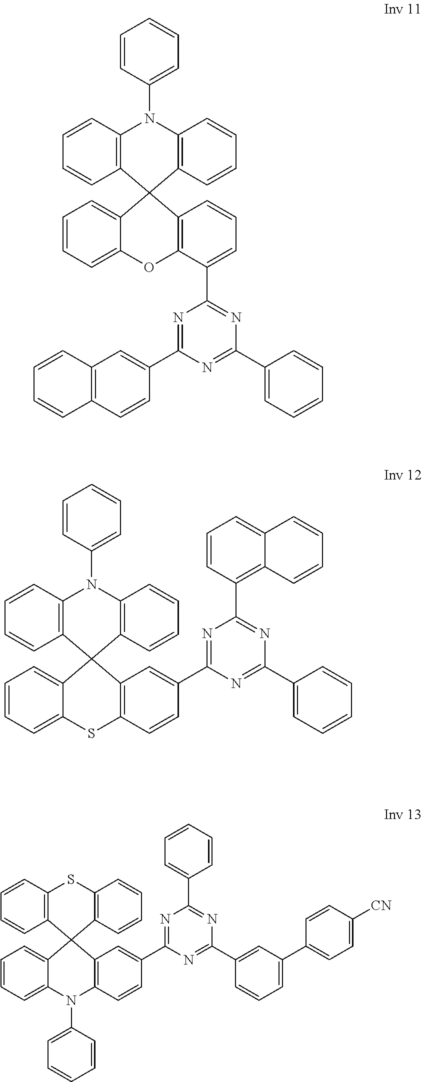 [SMILES: c1ccc(-c2nc(-c3ccc4ccccc4c3)nc(-c3cccc4c3Oc3ccccc3C43c4ccccc4N(c4ccccc4)c4ccccc43)n2)cc1 ; c1ccc(-c2nc(-c3ccc4c(c3)C3(c5ccccc5S4)c4ccccc4N(c4ccccc4)c4ccccc43)nc(-c3cccc4ccccc34)n2)cc1 ; N#Cc1ccc(-c2cccc(-c3nc(-c4ccccc4)nc(-c4ccc5c(c4)C4(c6ccccc6Sc6ccccc64)c4ccccc4N5c4ccccc4)n3)c2)cc1]

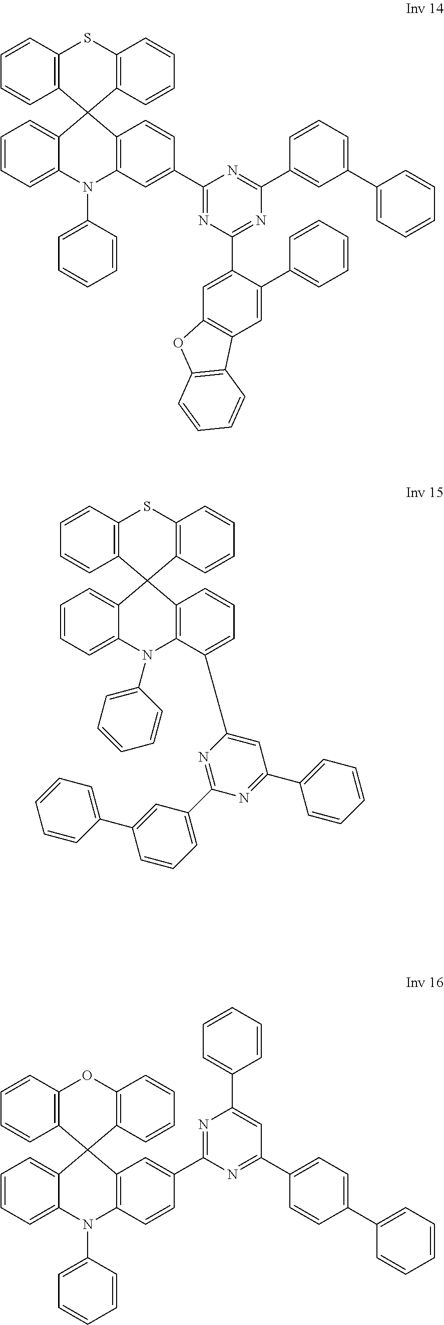 [SMILES: c1ccc(-c2cccc(-c3nc(-c4ccc5c(c4)N(c4ccccc4)c4ccccc4C54c5ccccc5Sc5ccccc54)nc(-c4cc5oc6ccccc6c5cc4-c4ccccc4)n3)c2)cc1 ; c1ccc(-c2cccc(-c3nc(-c4ccccc4)cc(-c4cccc5c4N(c4ccccc4)c4ccccc4C54c5ccccc5Sc5ccccc54)n3)c2)cc1 ; c1ccc(-c2ccc(-c3cc(-c4ccccc4)nc(-c4ccc5c(c4)C4(c6ccccc6Oc6ccccc64)c4ccccc4N5c4ccccc4)n3)cc2)cc1]

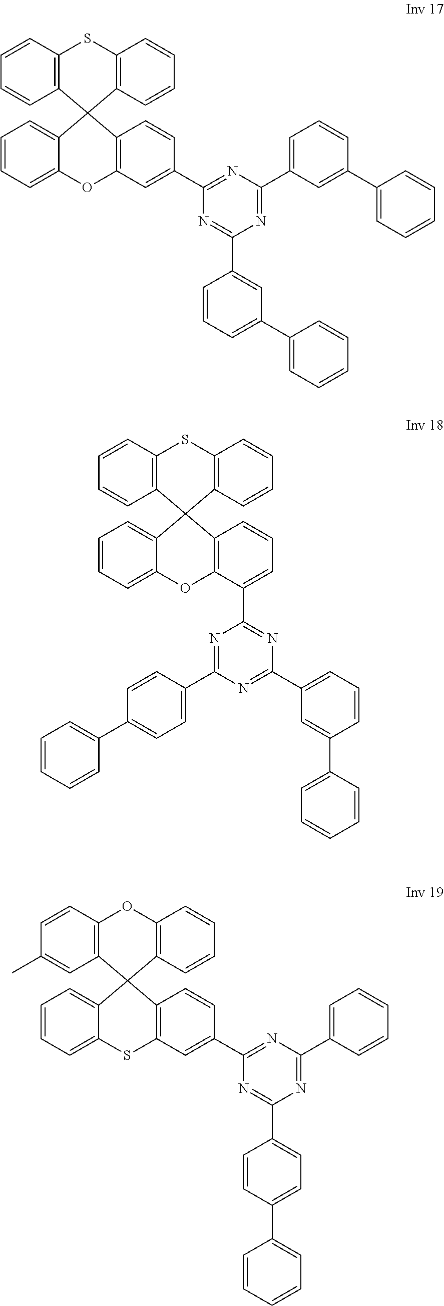 [SMILES: c1ccc(-c2cccc(-c3nc(-c4cccc(-c5ccccc5)c4)nc(-c4ccc5c(c4)Oc4ccccc4C54c5ccccc5Sc5ccccc54)n3)c2)cc1 ; c1ccc(-c2ccc(-c3nc(-c4cccc(-c5ccccc5)c4)nc(-c4cccc5c4Oc4ccccc4C54c5ccccc5Sc5ccccc54)n3)cc2)cc1 ; Cc1ccc2c(c1)C1(c3ccccc3O2)c2ccccc2Sc2cc(-c3nc(-c4ccccc4)nc(-c4ccc(-c5ccccc5)cc4)n3)ccc21]

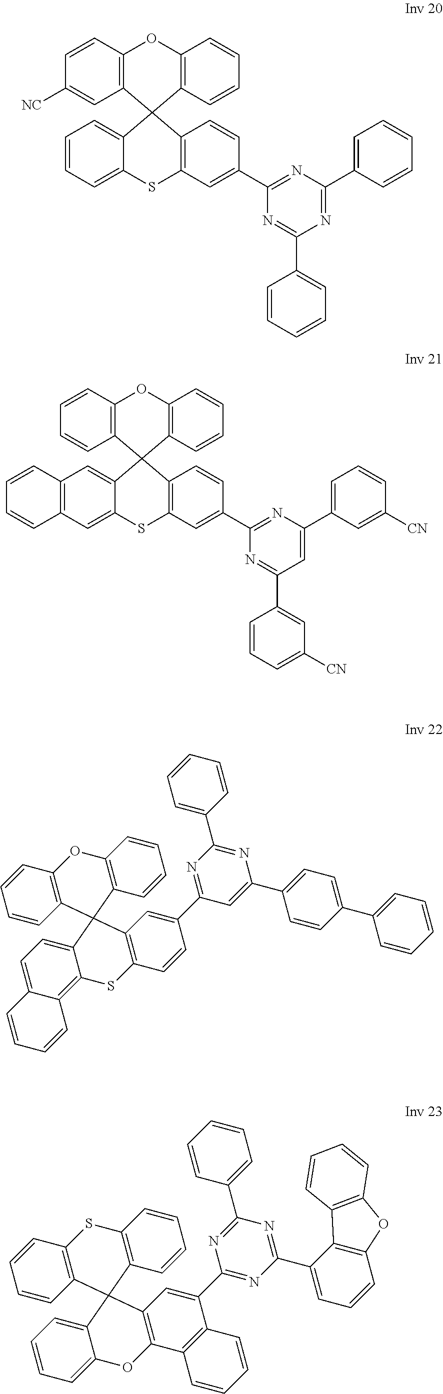 [SMILES: N#Cc1ccc2c(c1)C1(c3ccccc3O2)c2ccccc2Sc2cc(-c3nc(-c4ccccc4)nc(-c4ccccc4)n3)ccc21 ; N#Cc1cccc(-c2cc(-c3cccc(C#N)c3)nc(-c3ccc4c(c3)Sc3cc5ccccc5cc3C43c4ccccc4Oc4ccccc43)n2)c1 ; c1ccc(-c2ccc(-c3cc(-c4ccc5c(c4)C4(c6ccccc6Oc6ccccc64)c4ccc6ccccc6c4S5)nc(-c4ccccc4)n3)cc2)cc1 ; c1ccc(-c2nc(-c3cc4c(c5ccccc35)Oc3ccccc3C43c4ccccc4Sc4ccccc43)nc(-c3cccc4oc5ccccc5c34)n2)cc1]

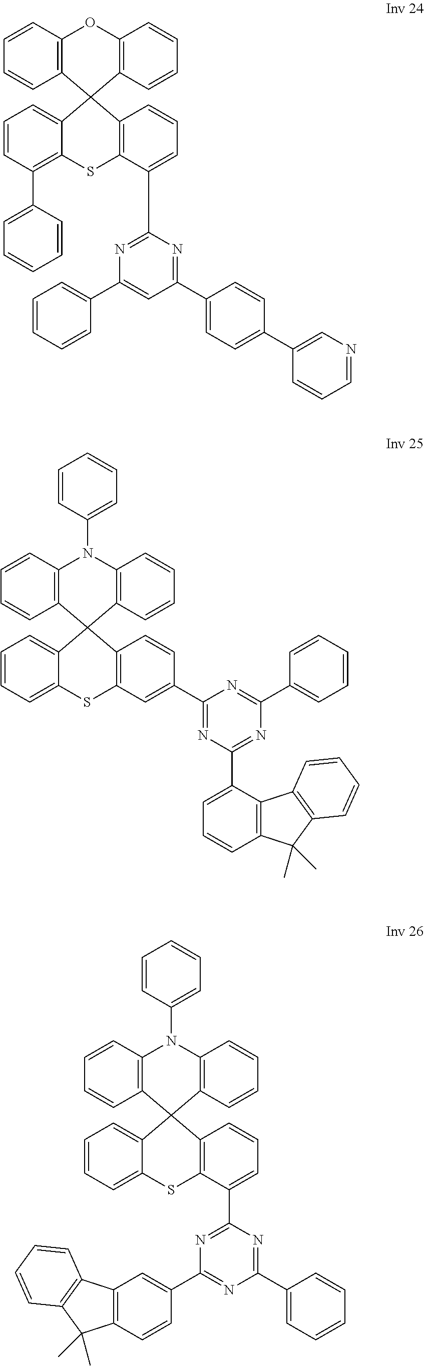 [SMILES: c1ccc(-c2cc(-c3ccc(-c4cccnc4)cc3)nc(-c3cccc4c3Sc3c(-c5ccccc5)cccc3C43c4ccccc4Oc4ccccc43)n2)cc1 ; CC1(C)c2ccccc2-c2c(-c3nc(-c4ccccc4)nc(-c4ccc5c(c4)Sc4ccccc4C54c5ccccc5N(c5ccccc5)c5ccccc54)n3)cccc21 ; CC1(C)c2ccccc2-c2cc(-c3nc(-c4ccccc4)nc(-c4cccc5c4Sc4ccccc4C54c5ccccc5N(c5ccccc5)c5ccccc54)n3)ccc21]

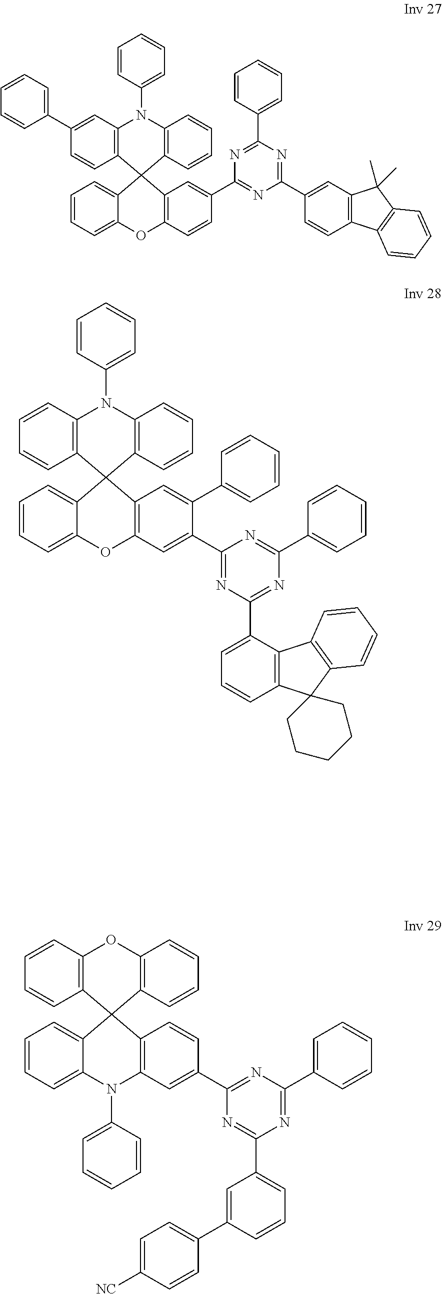 [SMILES: CC1(C)c2ccccc2-c2ccc(-c3nc(-c4ccccc4)nc(-c4ccc5c(c4)C4(c6ccccc6O5)c5ccccc5N(c5ccccc5)c5cc(-c6ccccc6)ccc54)n3)cc21 ; c1ccc(-c2nc(-c3cc4c(cc3-c3ccccc3)C3(c5ccccc5O4)c4ccccc4N(c4ccccc4)c4ccccc43)nc(-c3cccc4c3-c3ccccc3C43CCCCC3)n2)cc1 ; N#Cc1ccc(-c2cccc(-c3nc(-c4ccccc4)nc(-c4ccc5c(c4)N(c4ccccc4)c4ccccc4C54c5ccccc5Oc5ccccc54)n3)c2)cc1]

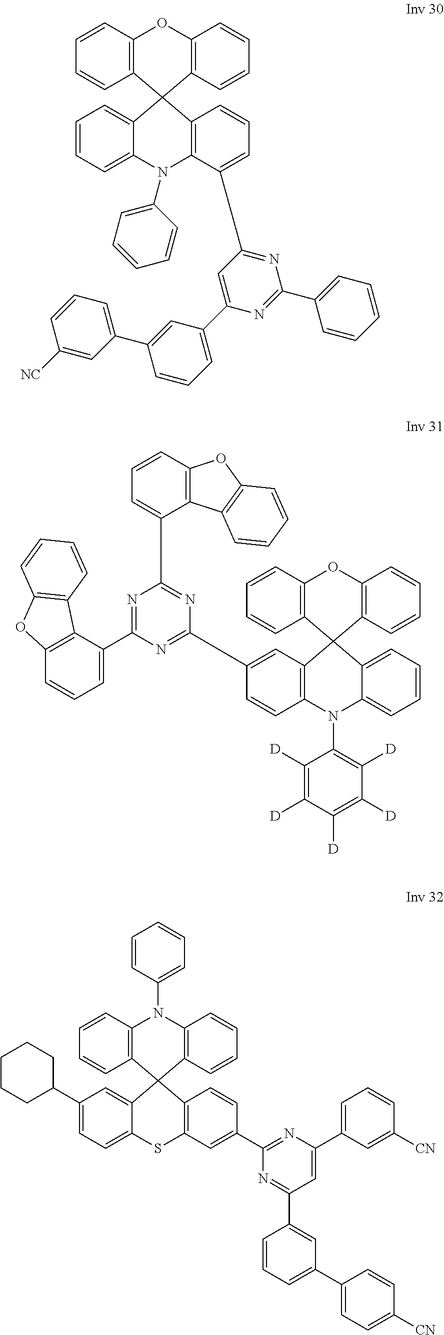 [SMILES: N#Cc1cccc(-c2cccc(-c3cc(-c4cccc5c4N(c4ccccc4)c4ccccc4C54c5ccccc5Oc5ccccc54)nc(-c4ccccc4)n3)c2)c1 ; [2H]c1c([2H])c([2H])c(N2c3ccccc3C3(c4ccccc4Oc4ccccc43)c3cc(-c4nc(-c5cccc6oc7ccccc7c56)nc(-c5cccc6oc7ccccc7c56)n4)ccc32)c([2H])c1[2H] ; N#Cc1ccc(-c2cccc(-c3cc(-c4cccc(C#N)c4)nc(-c4ccc5c(c4)Sc4ccc(C6CCCCC6)cc4C54c5ccccc5N(c5ccccc5)c5ccccc54)n3)c2)cc1]

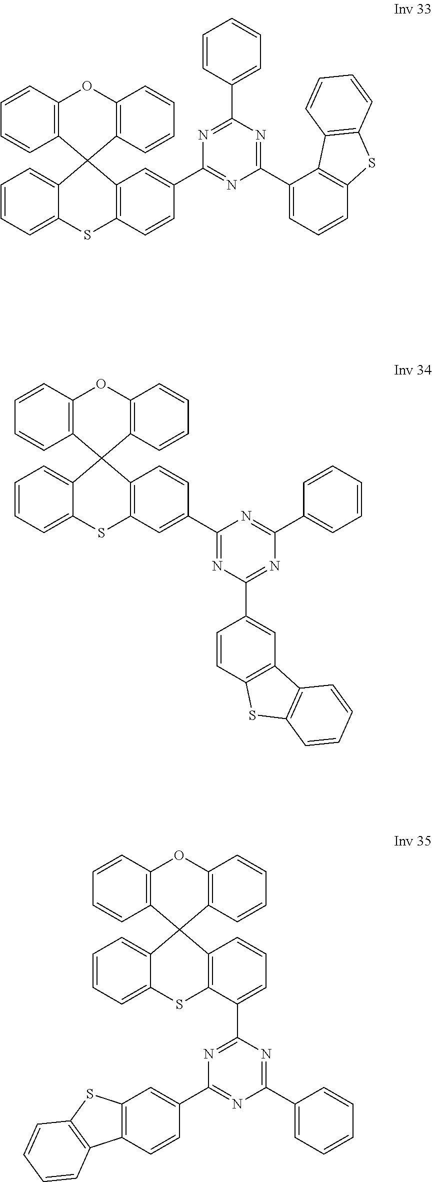 [SMILES: c1ccc(-c2nc(-c3ccc4c(c3)C3(c5ccccc5Oc5ccccc53)c3ccccc3S4)nc(-c3cccc4sc5ccccc5c34)n2)cc1 ; c1ccc(-c2nc(-c3ccc4c(c3)Sc3ccccc3C43c4ccccc4Oc4ccccc43)nc(-c3ccc4sc5ccccc5c4c3)n2)cc1 ; c1ccc(-c2nc(-c3ccc4c(c3)sc3ccccc34)nc(-c3cccc4c3Sc3ccccc3C43c4ccccc4Oc4ccccc43)n2)cc1]

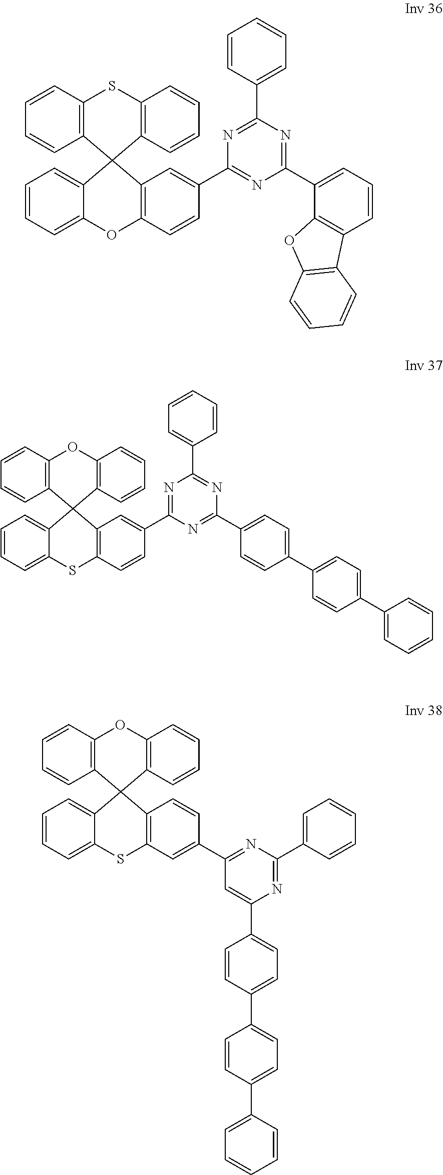 [SMILES: c1ccc(-c2nc(-c3ccc4c(c3)C3(c5ccccc5O4)c4ccccc4Sc4ccccc43)nc(-c3cccc4c3oc3ccccc34)n2)cc1 ; c1ccc(-c2ccc(-c3ccc(-c4nc(-c5ccccc5)nc(-c5ccc6c(c5)C5(c7ccccc7Oc7ccccc75)c5ccccc5S6)n4)cc3)cc2)cc1 ; c1ccc(-c2ccc(-c3ccc(-c4cc(-c5ccc6c(c5)Sc5ccccc5C65c6ccccc6Oc6ccccc65)nc(-c5ccccc5)n4)cc3)cc2)cc1]

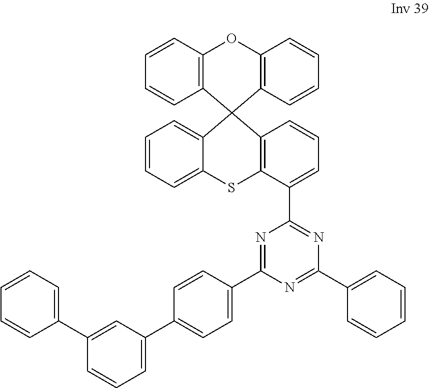 [SMILES: c1ccc(-c2cccc(-c3ccc(-c4nc(-c5ccccc5)nc(-c5cccc6c5Sc5ccccc5C65c6ccccc6Oc6ccccc65)n4)cc3)c2)cc1]

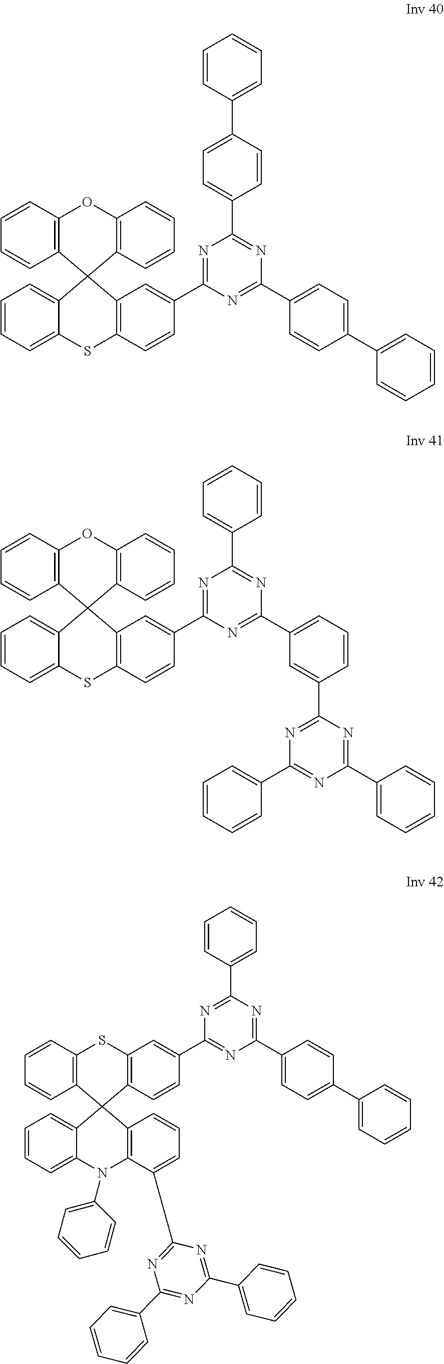 [SMILES: c1ccc(-c2ccc(-c3nc(-c4ccc(-c5ccccc5)cc4)nc(-c4ccc5c(c4)C4(c6ccccc6Oc6ccccc64)c4ccccc4S5)n3)cc2)cc1 ; c1ccc(-c2nc(-c3ccccc3)nc(-c3cccc(-c4nc(-c5ccccc5)nc(-c5ccc6c(c5)C5(c7ccccc7Oc7ccccc75)c5ccccc5S6)n4)c3)n2)cc1 ; c1ccc(-c2ccc(-c3nc(-c4ccccc4)nc(-c4ccc5c(c4)Sc4ccccc4C54c5ccccc5N(c5ccccc5)c5c(-c6nc(-c7ccccc7)nc(-c7ccccc7)n6)cccc54)n3)cc2)cc1]

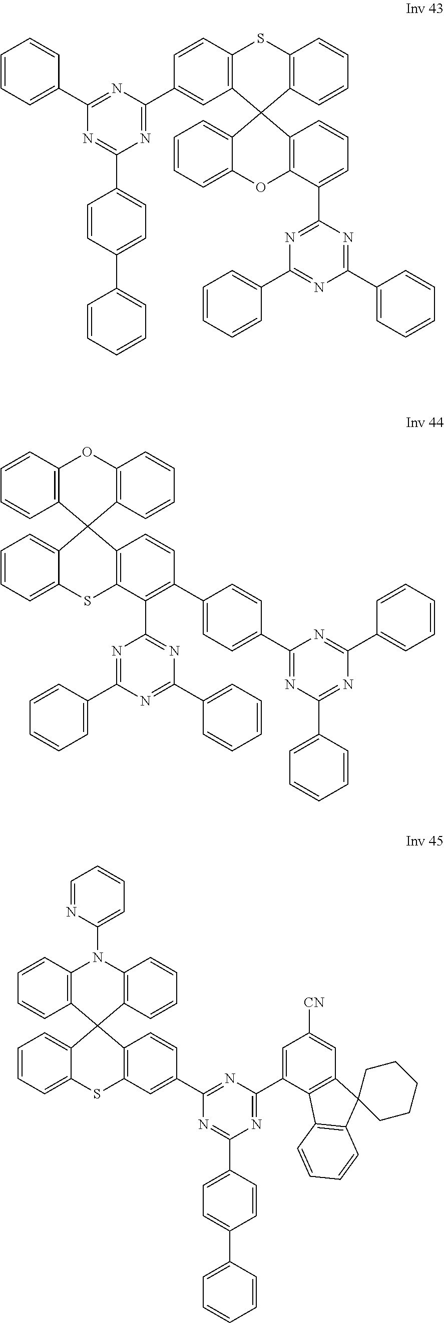 [SMILES: c1ccc(-c2ccc(-c3nc(-c4ccccc4)nc(-c4ccc5c(c4)C4(c6ccccc6Oc6c(-c7nc(-c8ccccc8)nc(-c8ccccc8)n7)cccc64)c4ccccc4S5)n3)cc2)cc1 ; c1ccc(-c2nc(-c3ccccc3)nc(-c3ccc(-c4ccc5c(c4-c4nc(-c6ccccc6)nc(-c6ccccc6)n4)Sc4ccccc4C54c5ccccc5Oc5ccccc54)cc3)n2)cc1 ; N#Cc1cc(-c2nc(-c3ccc(-c4ccccc4)cc3)nc(-c3ccc4c(c3)Sc3ccccc3C43c4ccccc4N(c4ccccn4)c4ccccc43)n2)c2c(c1)C1(CCCCC1)c1ccccc1-2]

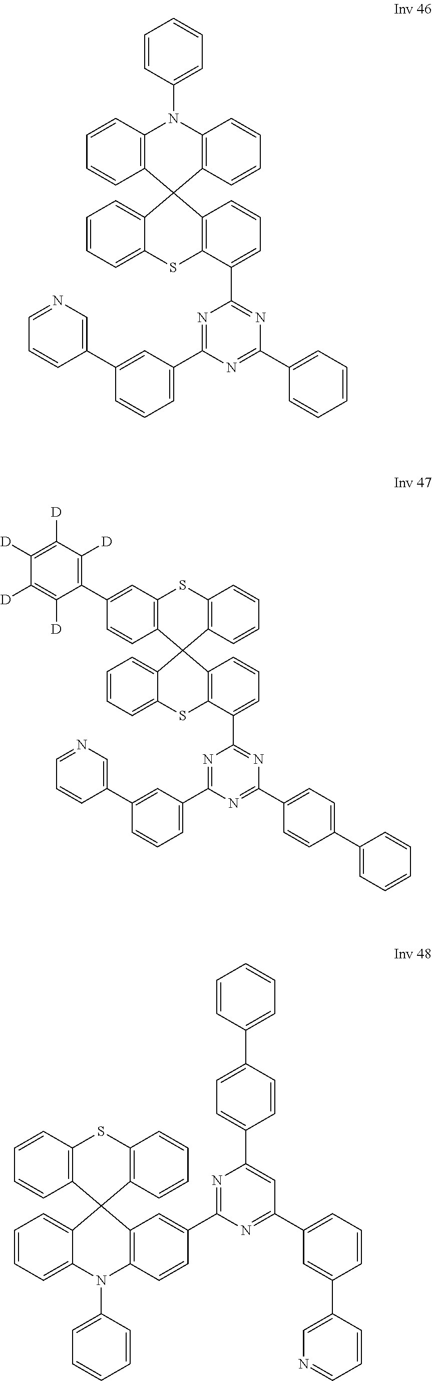 [SMILES: c1ccc(-c2nc(-c3cccc(-c4cccnc4)c3)nc(-c3cccc4c3Sc3ccccc3C43c4ccccc4N(c4ccccc4)c4ccccc43)n2)cc1 ; [2H]c1c([2H])c([2H])c(-c2ccc3c(c2)Sc2ccccc2C32c3ccccc3Sc3c(-c4nc(-c5ccc(-c6ccccc6)cc5)nc(-c5cccc(-c6cccnc6)c5)n4)cccc32)c([2H])c1[2H] ; c1ccc(-c2ccc(-c3cc(-c4cccc(-c5cccnc5)c4)nc(-c4ccc5c(c4)C4(c6ccccc6Sc6ccccc64)c4ccccc4N5c4ccccc4)n3)cc2)cc1]

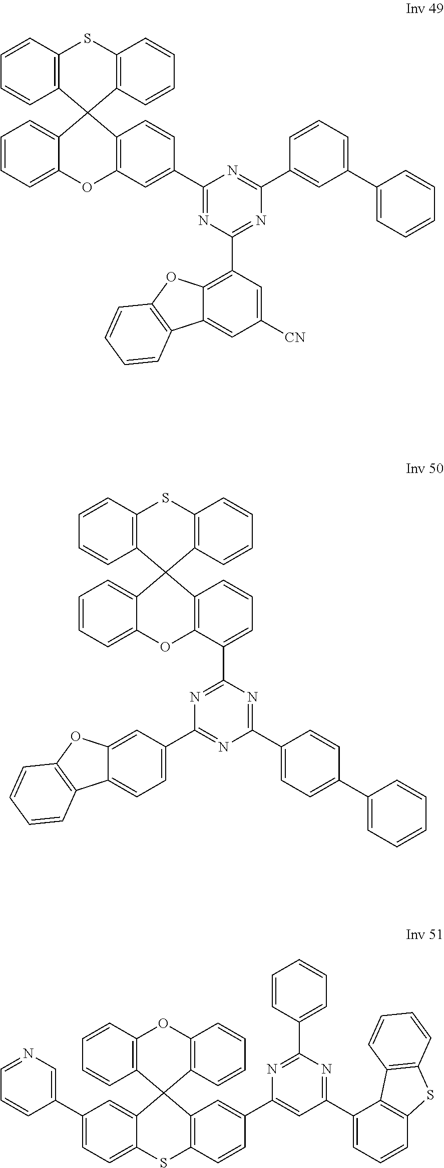 [SMILES: N#Cc1cc(-c2nc(-c3cccc(-c4ccccc4)c3)nc(-c3ccc4c(c3)Oc3ccccc3C43c4ccccc4Sc4ccccc43)n2)c2oc3ccccc3c2c1 ; c1ccc(-c2ccc(-c3nc(-c4ccc5c(c4)oc4ccccc45)nc(-c4cccc5c4Oc4ccccc4C54c5ccccc5Sc5ccccc54)n3)cc2)cc1 ; c1ccc(-c2nc(-c3ccc4c(c3)C3(c5ccccc5Oc5ccccc53)c3cc(-c5cccnc5)ccc3S4)cc(-c3cccc4sc5ccccc5c34)n2)cc1]

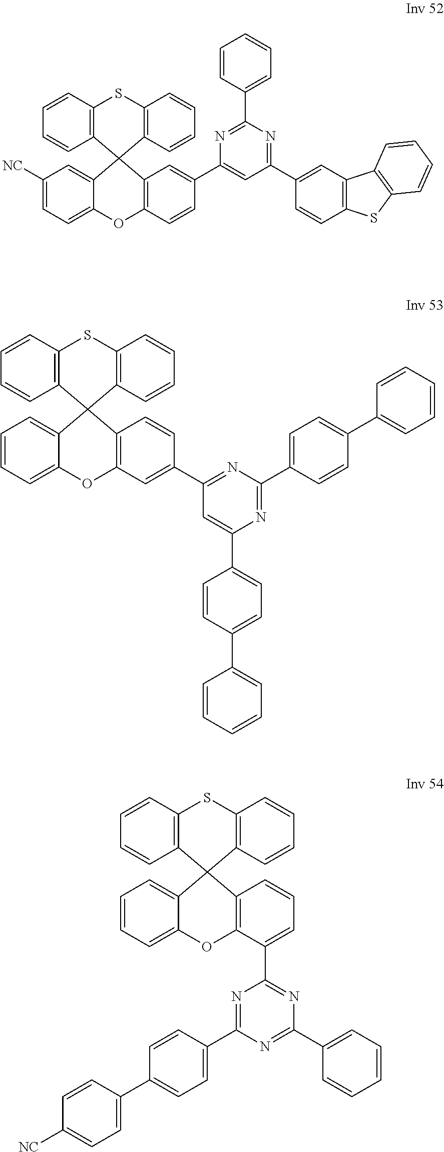 [SMILES: N#Cc1ccc2c(c1)C1(c3cc(-c4cc(-c5ccc6sc7ccccc7c6c5)nc(-c5ccccc5)n4)ccc3O2)c2ccccc2Sc2ccccc21 ; c1ccc(-c2ccc(-c3cc(-c4ccc5c(c4)Oc4ccccc4C54c5ccccc5Sc5ccccc54)nc(-c4ccc(-c5ccccc5)cc4)n3)cc2)cc1 ; N#Cc1ccc(-c2ccc(-c3nc(-c4ccccc4)nc(-c4cccc5c4Oc4ccccc4C54c5ccccc5Sc5ccccc54)n3)cc2)cc1]

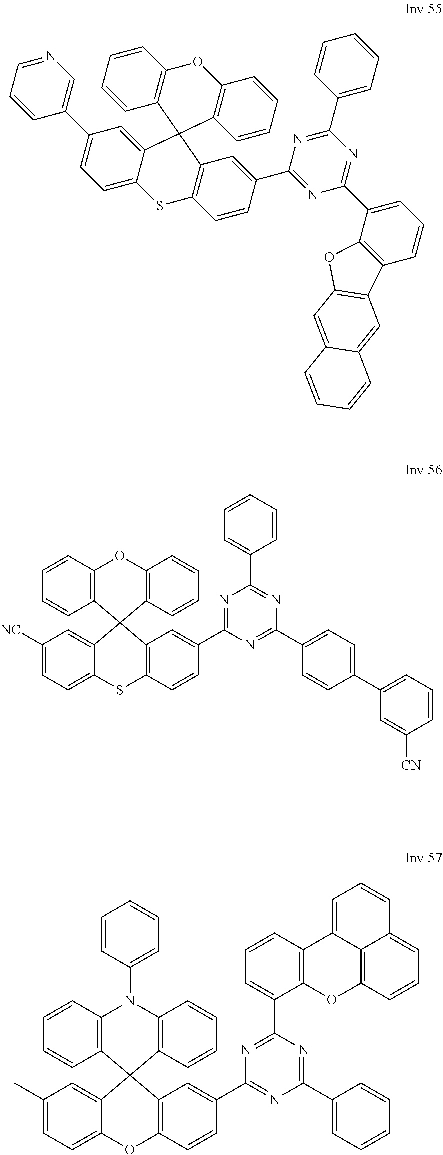 [SMILES: c1ccc(-c2nc(-c3ccc4c(c3)C3(c5ccccc5Oc5ccccc53)c3cc(-c5cccnc5)ccc3S4)nc(-c3cccc4c3oc3cc5ccccc5cc34)n2)cc1 ; N#Cc1cccc(-c2ccc(-c3nc(-c4ccccc4)nc(-c4ccc5c(c4)C4(c6ccccc6Oc6ccccc64)c4cc(C#N)ccc4S5)n3)cc2)c1 ; Cc1ccc2c(c1)C1(c3cc(-c4nc(-c5ccccc5)nc(-c5cccc6c5Oc5cccc7cccc-6c57)n4)ccc3O2)c2ccccc2N(c2ccccc2)c2ccccc21]

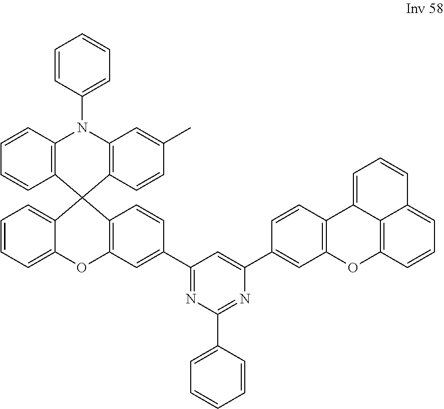 [SMILES: Cc1ccc2c(c1)N(c1ccccc1)c1ccccc1C21c2ccccc2Oc2cc(-c3cc(-c4ccc5c(c4)Oc4cccc6cccc-5c46)nc(-c4ccccc4)n3)ccc21]

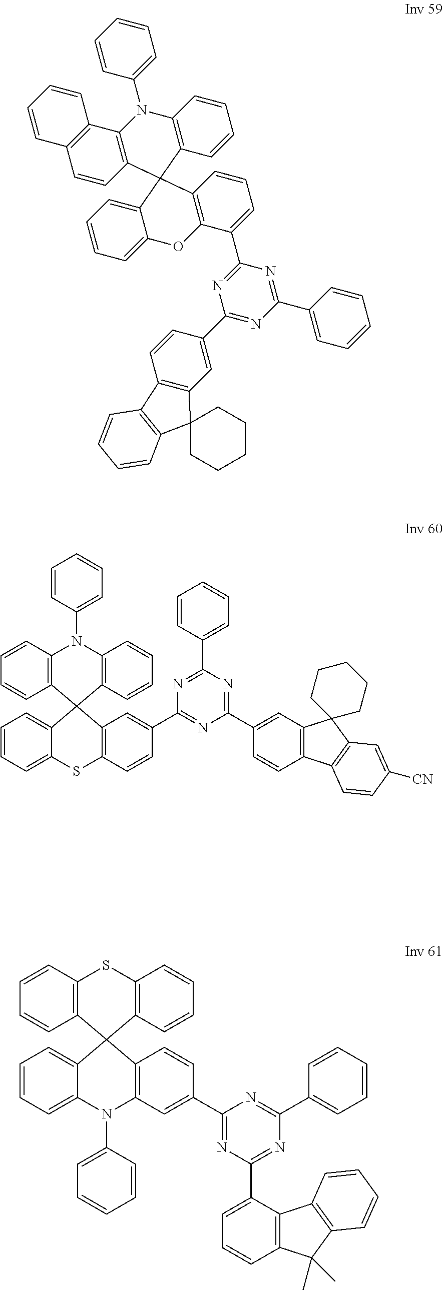 [SMILES: c1ccc(-c2nc(-c3ccc4c(c3)C3(CCCCC3)c3ccccc3-4)nc(-c3cccc4c3Oc3ccccc3C43c4ccccc4N(c4ccccc4)c4c3ccc3ccccc43)n2)cc1 ; N#Cc1ccc2c(c1)C1(CCCCC1)c1cc(-c3nc(-c4ccccc4)nc(-c4ccc5c(c4)C4(c6ccccc6S5)c5ccccc5N(c5ccccc5)c5ccccc54)n3)ccc1-2 ; CC1(C)c2ccccc2-c2c(-c3nc(-c4ccccc4)nc(-c4ccc5c(c4)N(c4ccccc4)c4ccccc4C54c5ccccc5Sc5ccccc54)n3)cccc21]

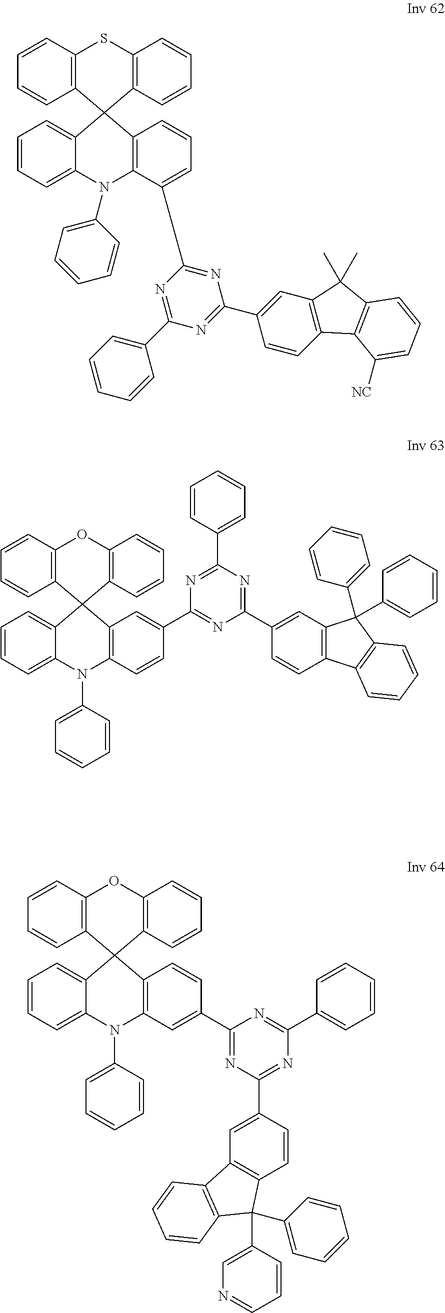 [SMILES: CC1(C)c2cc(-c3nc(-c4ccccc4)nc(-c4cccc5c4N(c4ccccc4)c4ccccc4C54c5ccccc5Sc5ccccc54)n3)ccc2-c2c(C#N)cccc21 ; c1ccc(-c2nc(-c3ccc4c(c3)C(c3ccccc3)(c3ccccc3)c3ccccc3-4)nc(-c3ccc4c(c3)C3(c5ccccc5Oc5ccccc53)c3ccccc3N4c3ccccc3)n2)cc1 ; c1ccc(-c2nc(-c3ccc4c(c3)-c3ccccc3C4(c3ccccc3)c3cccnc3)nc(-c3ccc4c(c3)N(c3ccccc3)c3ccccc3C43c4ccccc4Oc4ccccc43)n2)cc1]

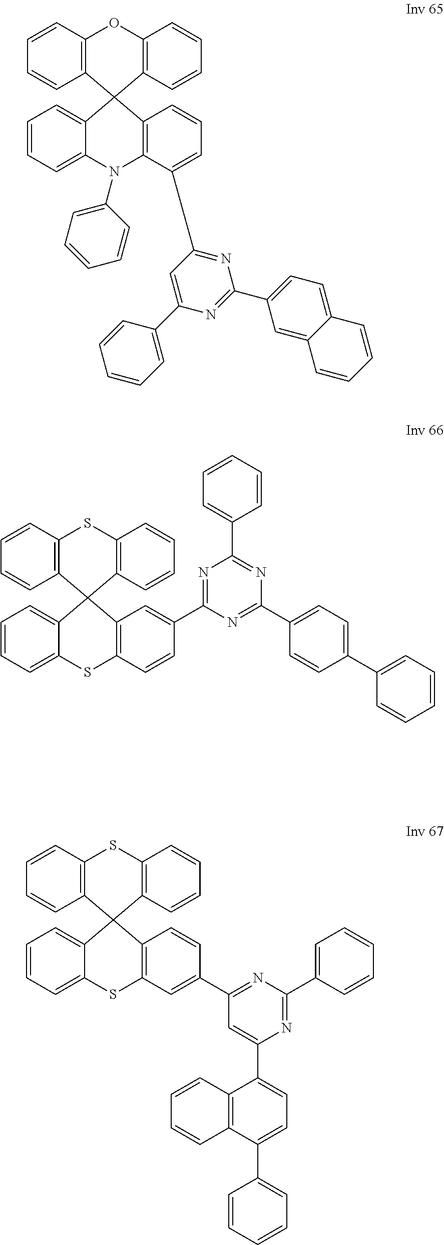 [SMILES: c1ccc(-c2cc(-c3cccc4c3N(c3ccccc3)c3ccccc3C43c4ccccc4Oc4ccccc43)nc(-c3ccc4ccccc4c3)n2)cc1 ; c1ccc(-c2ccc(-c3nc(-c4ccccc4)nc(-c4ccc5c(c4)C4(c6ccccc6Sc6ccccc64)c4ccccc4S5)n3)cc2)cc1 ; c1ccc(-c2nc(-c3ccc4c(c3)Sc3ccccc3C43c4ccccc4Sc4ccccc43)cc(-c3ccc(-c4ccccc4)c4ccccc34)n2)cc1]

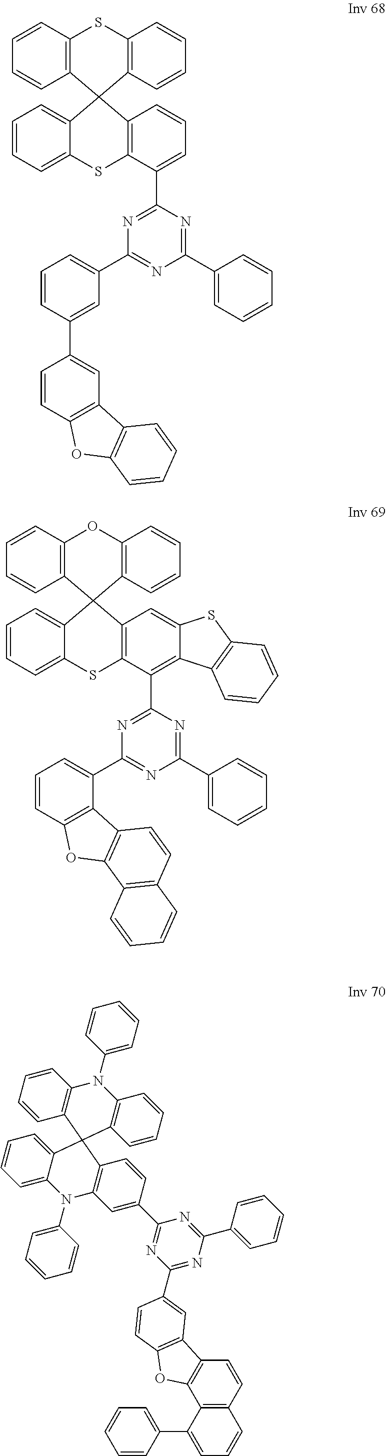

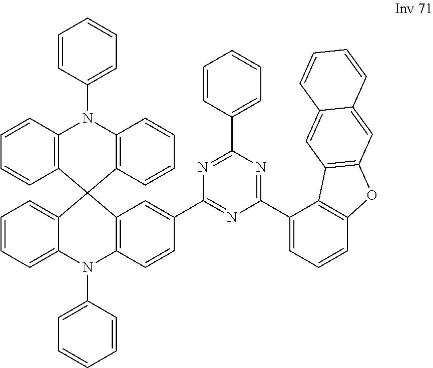 [SMILES: c1ccc(-c2nc(-c3ccc4c(c3)C3(c5ccccc5N(c5ccccc5)c5ccccc53)c3ccccc3N4c3ccccc3)nc(-c3cccc4oc5cc6ccccc6cc5c34)n2)cc1]

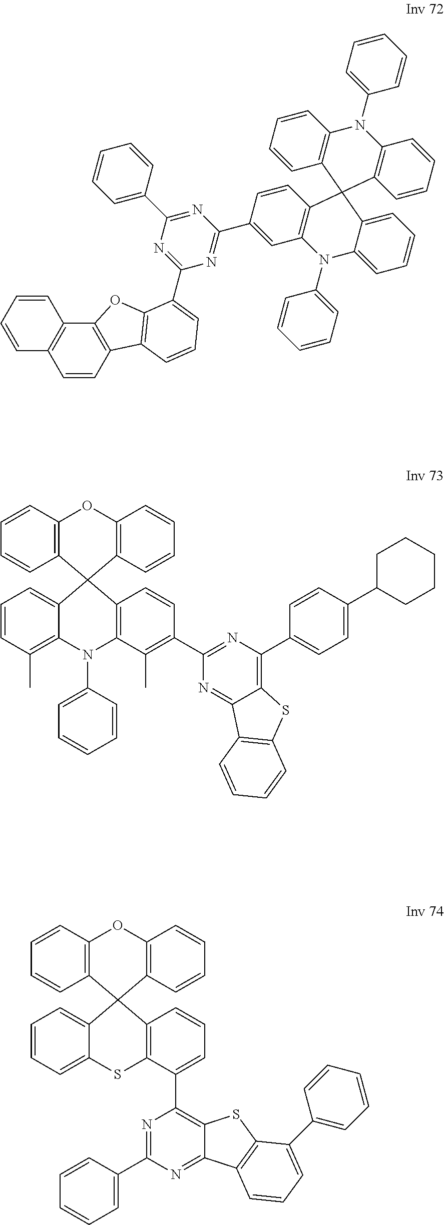 [SMILES: c1ccc(-c2nc(-c3ccc4c(c3)N(c3ccccc3)c3ccccc3C43c4ccccc4N(c4ccccc4)c4ccccc43)nc(-c3cccc4c3oc3c5ccccc5ccc43)n2)cc1 ; Cc1cccc2c1N(c1ccccc1)c1c(ccc(-c3nc(-c4ccc(C5CCCCC5)cc4)c4sc5ccccc5c4n3)c1C)C21c2ccccc2Oc2ccccc21 ; c1ccc(-c2nc(-c3cccc4c3Sc3ccccc3C43c4ccccc4Oc4ccccc43)c3sc4c(-c5ccccc5)cccc4c3n2)cc1]

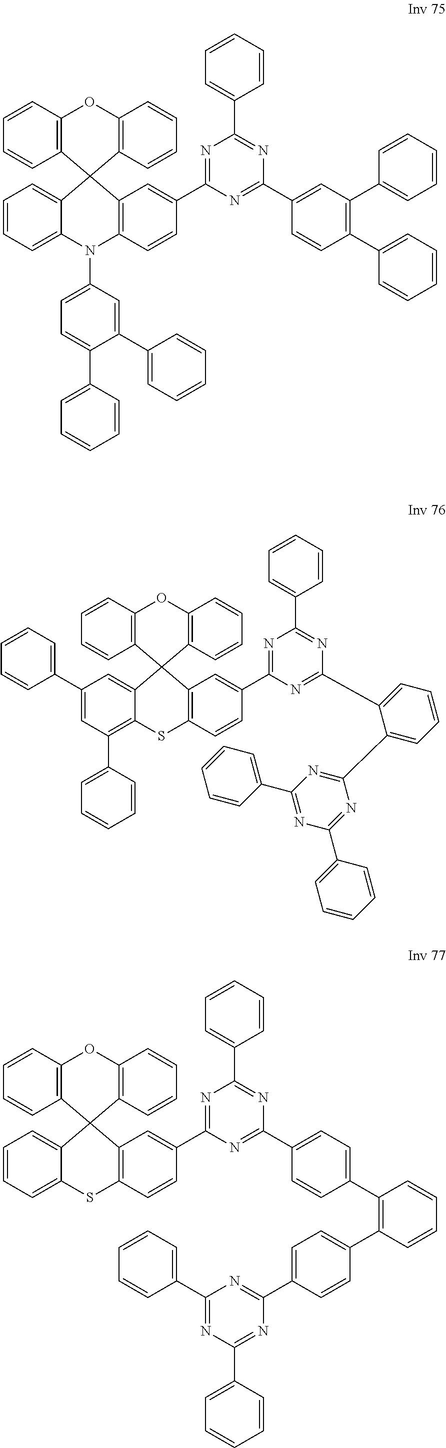 [SMILES: c1ccc(-c2nc(-c3ccc(-c4ccccc4)c(-c4ccccc4)c3)nc(-c3ccc4c(c3)C3(c5ccccc5Oc5ccccc53)c3ccccc3N4c3ccc(-c4ccccc4)c(-c4ccccc4)c3)n2)cc1 ; c1ccc(-c2cc(-c3ccccc3)c3c(c2)C2(c4ccccc4Oc4ccccc42)c2cc(-c4nc(-c5ccccc5)nc(-c5ccccc5-c5nc(-c6ccccc6)nc(-c6ccccc6)n5)n4)ccc2S3)cc1 ; c1ccc(-c2nc(-c3ccccc3)nc(-c3ccc(-c4ccccc4-c4ccc(-c5nc(-c6ccccc6)nc(-c6ccc7c(c6)C6(c8ccccc8Oc8ccccc86)c6ccccc6S7)n5)cc4)cc3)n2)cc1]

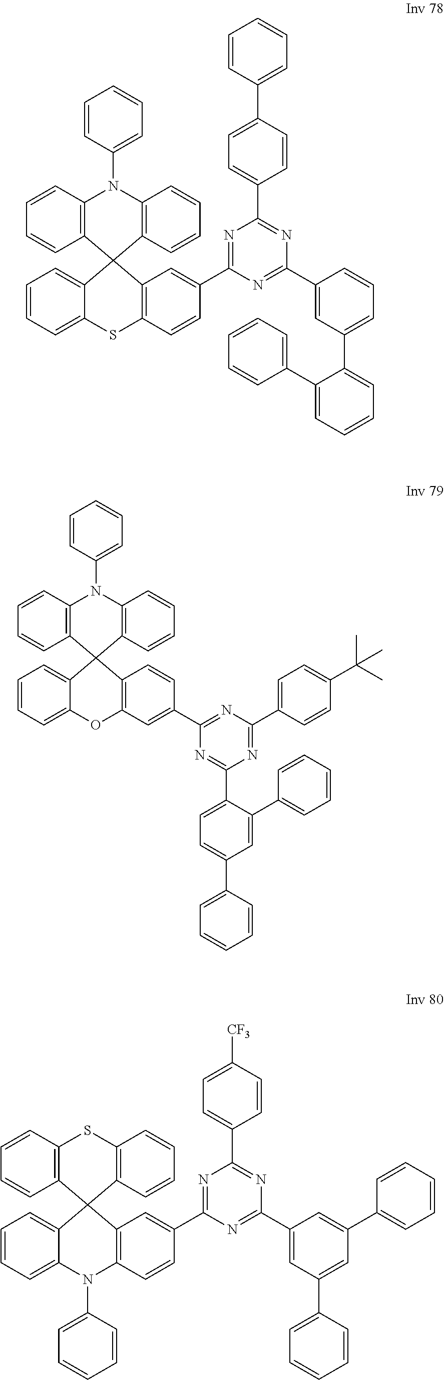 [SMILES: c1ccc(-c2ccc(-c3nc(-c4cccc(-c5ccccc5-c5ccccc5)c4)nc(-c4ccc5c(c4)C4(c6ccccc6S5)c5ccccc5N(c5ccccc5)c5ccccc54)n3)cc2)cc1 ; CC(C)(C)c1ccc(-c2nc(-c3ccc4c(c3)Oc3ccccc3C43c4ccccc4N(c4ccccc4)c4ccccc43)nc(-c3ccc(-c4ccccc4)cc3-c3ccccc3)n2)cc1 ; FC(F)(F)c1ccc(-c2nc(-c3cc(-c4ccccc4)cc(-c4ccccc4)c3)nc(-c3ccc4c(c3)C3(c5ccccc5Sc5ccccc53)c3ccccc3N4c3ccccc3)n2)cc1]

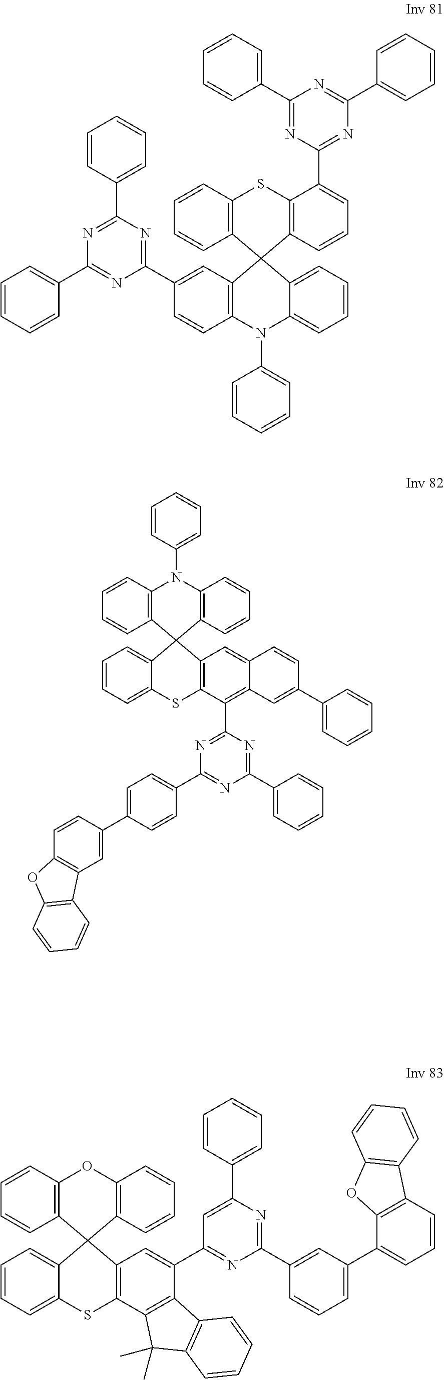 [SMILES: c1ccc(-c2nc(-c3ccccc3)nc(-c3ccc4c(c3)C3(c5ccccc5Sc5c(-c6nc(-c7ccccc7)nc(-c7ccccc7)n6)cccc53)c3ccccc3N4c3ccccc3)n2)cc1 ; c1ccc(-c2ccc3cc4c(c(-c5nc(-c6ccccc6)nc(-c6ccc(-c7ccc8oc9ccccc9c8c7)cc6)n5)c3c2)Sc2ccccc2C42c3ccccc3N(c3ccccc3)c3ccccc32)cc1 ; CC1(C)c2ccccc2-c2c(-c3cc(-c4ccccc4)nc(-c4cccc(-c5cccc6c5oc5ccccc56)c4)n3)cc3c(c21)Sc1ccccc1C31c2ccccc2Oc2ccccc21]

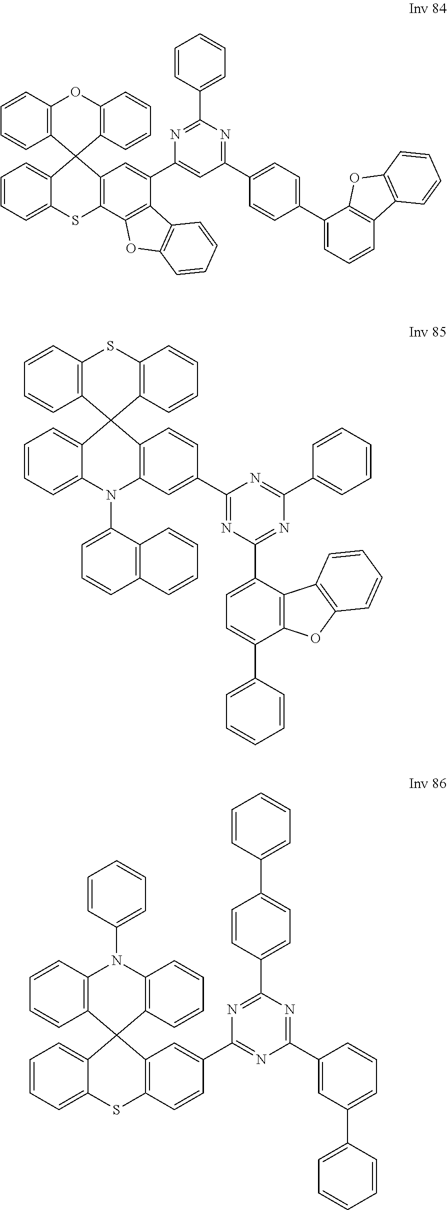 [SMILES: c1ccc(-c2nc(-c3ccc(-c4cccc5c4oc4ccccc45)cc3)cc(-c3cc4c(c5oc6ccccc6c35)Sc3ccccc3C43c4ccccc4Oc4ccccc43)n2)cc1 ; c1ccc(-c2nc(-c3ccc4c(c3)N(c3cccc5ccccc35)c3ccccc3C43c4ccccc4Sc4ccccc43)nc(-c3ccc(-c4ccccc4)c4oc5ccccc5c34)n2)cc1 ; c1ccc(-c2ccc(-c3nc(-c4cccc(-c5ccccc5)c4)nc(-c4ccc5c(c4)C4(c6ccccc6S5)c5ccccc5N(c5ccccc5)c5ccccc54)n3)cc2)cc1]

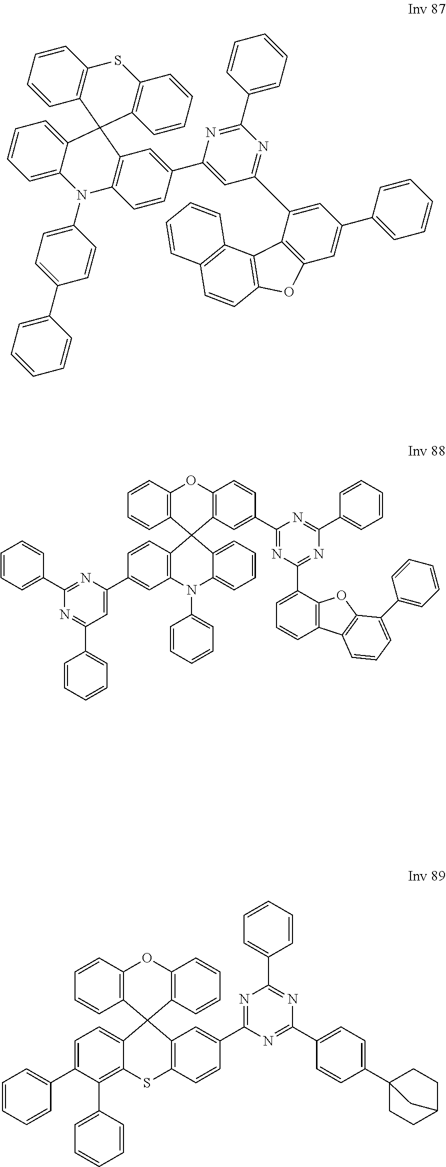 [SMILES: c1ccc(-c2ccc(N3c4ccccc4C4(c5ccccc5Sc5ccccc54)c4cc(-c5cc(-c6cc(-c7ccccc7)cc7oc8ccc9ccccc9c8c67)nc(-c6ccccc6)n5)ccc43)cc2)cc1 ; c1ccc(-c2cc(-c3ccc4c(c3)N(c3ccccc3)c3ccccc3C43c4ccccc4Oc4ccc(-c5nc(-c6ccccc6)nc(-c6cccc7c6oc6c(-c8ccccc8)cccc67)n5)cc43)nc(-c3ccccc3)n2)cc1 ; c1ccc(-c2nc(-c3ccc(C45CCC(CC4)C5)cc3)nc(-c3ccc4c(c3)C3(c5ccccc5Oc5ccccc53)c3ccc(-c5ccccc5)c(-c5ccccc5)c3S4)n2)cc1]

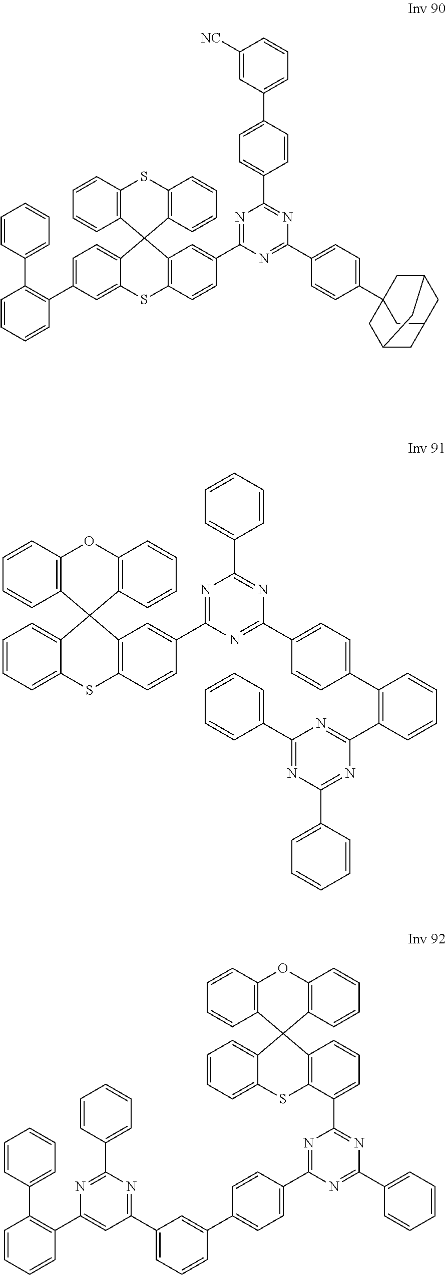 [SMILES: N#Cc1cccc(-c2ccc(-c3nc(-c4ccc(C56CC7CC(CC(C7)C5)C6)cc4)nc(-c4ccc5c(c4)C4(c6ccccc6Sc6ccccc64)c4ccc(-c6ccccc6-c6ccccc6)cc4S5)n3)cc2)c1 ; c1ccc(-c2nc(-c3ccc(-c4ccccc4-c4nc(-c5ccccc5)nc(-c5ccccc5)n4)cc3)nc(-c3ccc4c(c3)C3(c5ccccc5Oc5ccccc53)c3ccccc3S4)n2)cc1 ; c1ccc(-c2nc(-c3cccc(-c4ccc(-c5nc(-c6ccccc6)nc(-c6cccc7c6Sc6ccccc6C76c7ccccc7Oc7ccccc76)n5)cc4)c3)cc(-c3ccccc3-c3ccccc3)n2)cc1]

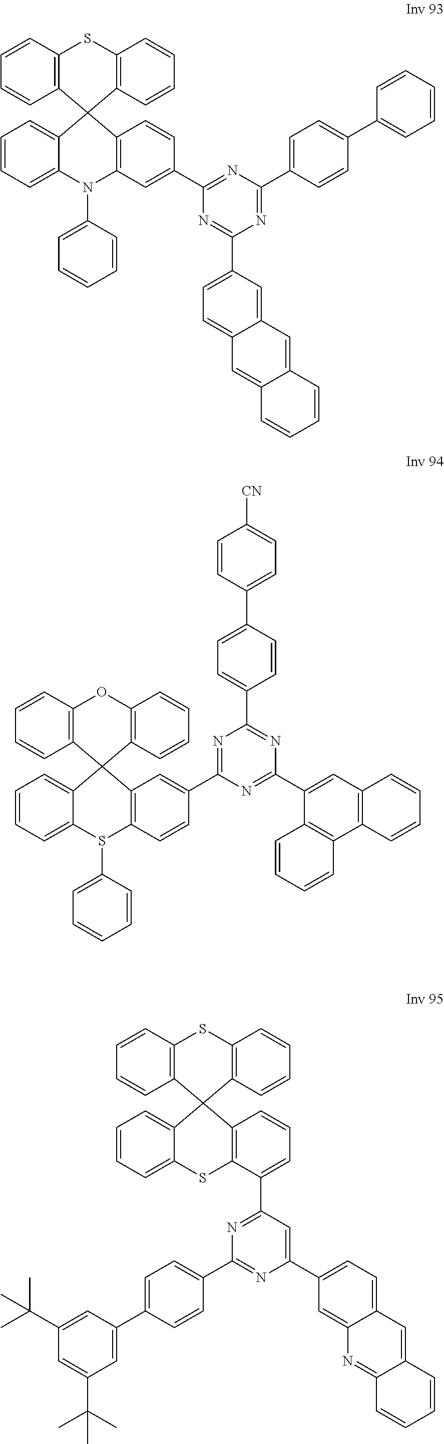

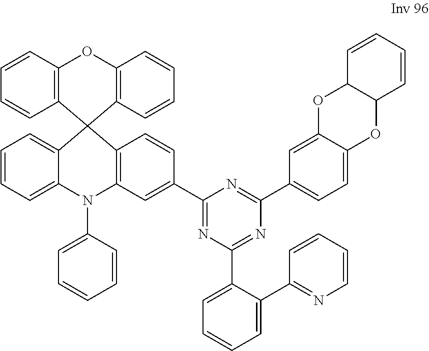 [SMILES: C1=CC2Oc3ccc(-c4nc(-c5ccc6c(c5)N(c5ccccc5)c5ccccc5C65c6ccccc6Oc6ccccc65)nc(-c5ccccc5-c5ccccn5)n4)cc3OC2C=C1]

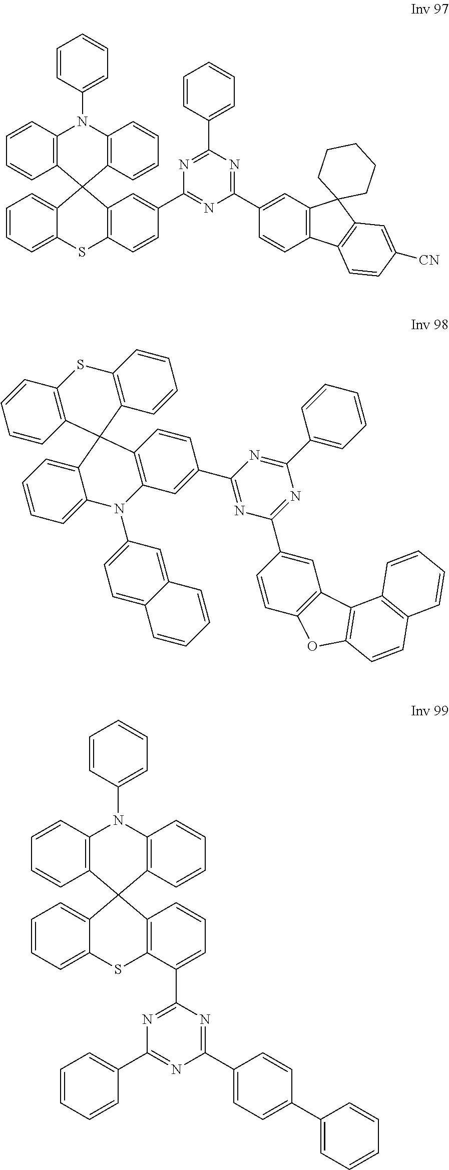 [SMILES: N#Cc1ccc2c(c1)C1(CCCCC1)c1cc(-c3nc(-c4ccccc4)nc(-c4ccc5c(c4)C4(c6ccccc6S5)c5ccccc5N(c5ccccc5)c5ccccc54)n3)ccc1-2 ; c1ccc(-c2nc(-c3ccc4c(c3)N(c3ccc5ccccc5c3)c3ccccc3C43c4ccccc4Sc4ccccc43)nc(-c3ccc4oc5ccc6ccccc6c5c4c3)n2)cc1 ; c1ccc(-c2ccc(-c3nc(-c4ccccc4)nc(-c4cccc5c4Sc4ccccc4C54c5ccccc5N(c5ccccc5)c5ccccc54)n3)cc2)cc1]

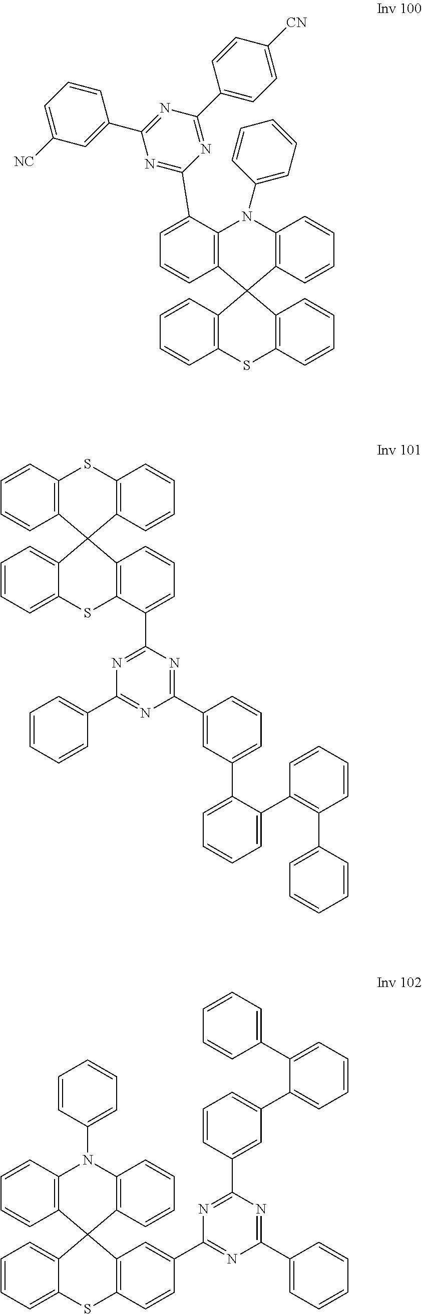 [SMILES: N#Cc1ccc(-c2nc(-c3cccc(C#N)c3)nc(-c3cccc4c3N(c3ccccc3)c3ccccc3C43c4ccccc4Sc4ccccc43)n2)cc1 ; c1ccc(-c2nc(-c3cccc(-c4ccccc4-c4ccccc4-c4ccccc4)c3)nc(-c3cccc4c3Sc3ccccc3C43c4ccccc4Sc4ccccc43)n2)cc1 ; c1ccc(-c2nc(-c3cccc(-c4ccccc4-c4ccccc4)c3)nc(-c3ccc4c(c3)C3(c5ccccc5S4)c4ccccc4N(c4ccccc4)c4ccccc43)n2)cc1]

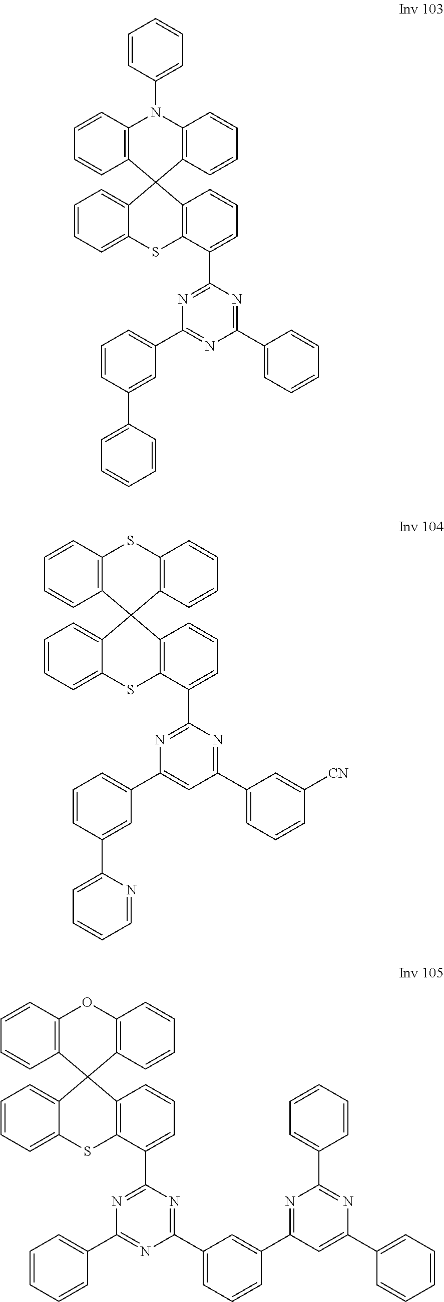 [SMILES: c1ccc(-c2cccc(-c3nc(-c4ccccc4)nc(-c4cccc5c4Sc4ccccc4C54c5ccccc5N(c5ccccc5)c5ccccc54)n3)c2)cc1 ; N#Cc1cccc(-c2cc(-c3cccc(-c4ccccn4)c3)nc(-c3cccc4c3Sc3ccccc3C43c4ccccc4Sc4ccccc43)n2)c1 ; c1ccc(-c2cc(-c3cccc(-c4nc(-c5ccccc5)nc(-c5cccc6c5Sc5ccccc5C65c6ccccc6Oc6ccccc65)n4)c3)nc(-c3ccccc3)n2)cc1]

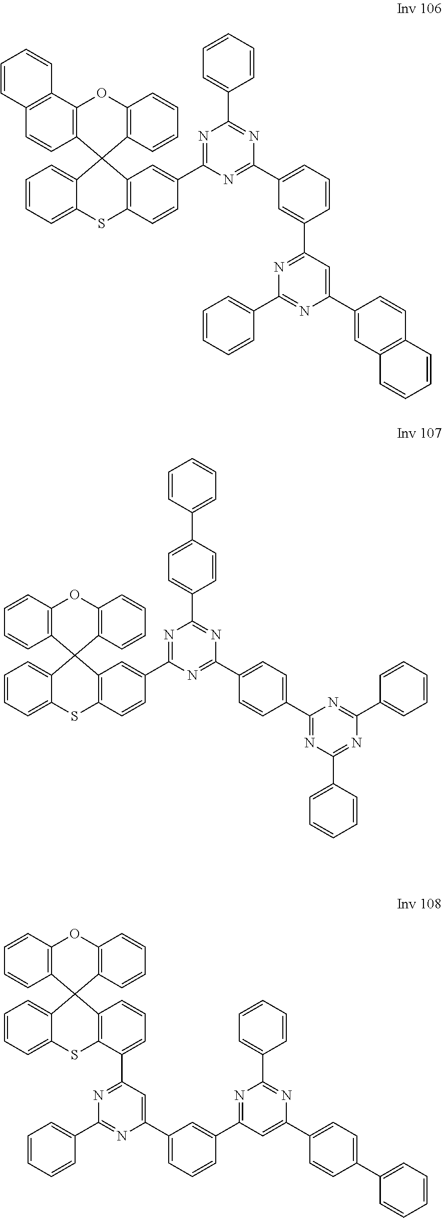 [SMILES: c1ccc(-c2nc(-c3cccc(-c4nc(-c5ccccc5)nc(-c5ccc6c(c5)C5(c7ccccc7Oc7c5ccc5ccccc75)c5ccccc5S6)n4)c3)cc(-c3ccc4ccccc4c3)n2)cc1 ; c1ccc(-c2ccc(-c3nc(-c4ccc(-c5nc(-c6ccccc6)nc(-c6ccccc6)n5)cc4)nc(-c4ccc5c(c4)C4(c6ccccc6Oc6ccccc64)c4ccccc4S5)n3)cc2)cc1 ; c1ccc(-c2ccc(-c3cc(-c4cccc(-c5cc(-c6cccc7c6Sc6ccccc6C76c7ccccc7Oc7ccccc76)nc(-c6ccccc6)n5)c4)nc(-c4ccccc4)n3)cc2)cc1]

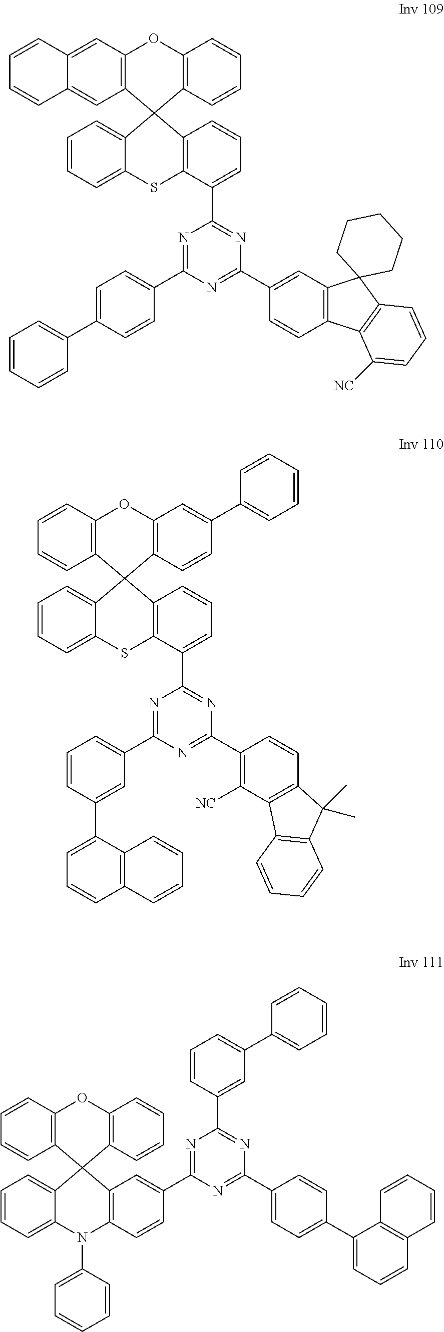 [SMILES: N#Cc1cccc2c1-c1ccc(-c3nc(-c4ccc(-c5ccccc5)cc4)nc(-c4cccc5c4Sc4ccccc4C54c5ccccc5Oc5cc6ccccc6cc54)n3)cc1C21CCCCC1 ; CC1(C)c2ccccc2-c2c1ccc(-c1nc(-c3cccc(-c4cccc5ccccc45)c3)nc(-c3cccc4c3Sc3ccccc3C43c4ccccc4Oc4cc(-c5ccccc5)ccc43)n1)c2C#N ; c1ccc(-c2cccc(-c3nc(-c4ccc(-c5cccc6ccccc56)cc4)nc(-c4ccc5c(c4)C4(c6ccccc6Oc6ccccc64)c4ccccc4N5c4ccccc4)n3)c2)cc1]

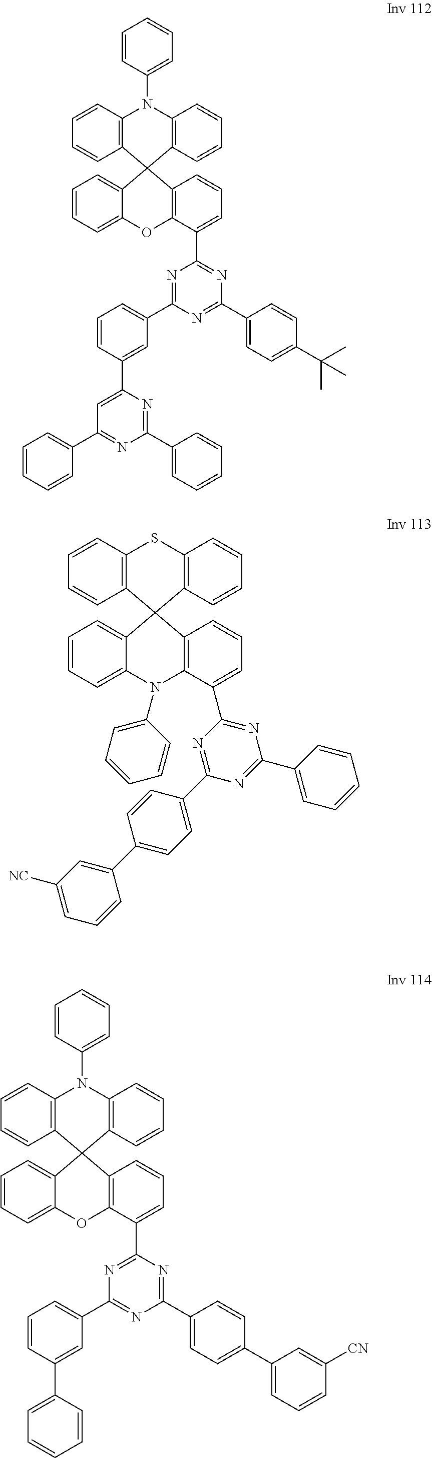 [SMILES: CC(C)(C)c1ccc(-c2nc(-c3cccc(-c4cc(-c5ccccc5)nc(-c5ccccc5)n4)c3)nc(-c3cccc4c3Oc3ccccc3C43c4ccccc4N(c4ccccc4)c4ccccc43)n2)cc1 ; N#Cc1cccc(-c2ccc(-c3nc(-c4ccccc4)nc(-c4cccc5c4N(c4ccccc4)c4ccccc4C54c5ccccc5Sc5ccccc54)n3)cc2)c1 ; N#Cc1cccc(-c2ccc(-c3nc(-c4cccc(-c5ccccc5)c4)nc(-c4cccc5c4Oc4ccccc4C54c5ccccc5N(c5ccccc5)c5ccccc54)n3)cc2)c1]

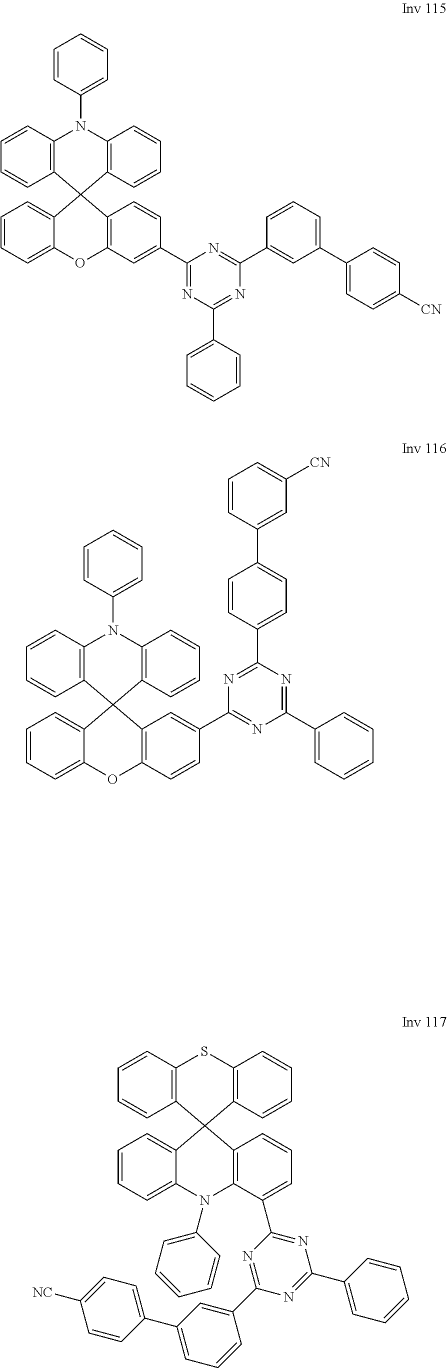 [SMILES: N#Cc1ccc(-c2cccc(-c3nc(-c4ccccc4)nc(-c4ccc5c(c4)Oc4ccccc4C54c5ccccc5N(c5ccccc5)c5ccccc54)n3)c2)cc1 ; N#Cc1cccc(-c2ccc(-c3nc(-c4ccccc4)nc(-c4ccc5c(c4)C4(c6ccccc6O5)c5ccccc5N(c5ccccc5)c5ccccc54)n3)cc2)c1 ; N#Cc1ccc(-c2cccc(-c3nc(-c4ccccc4)nc(-c4cccc5c4N(c4ccccc4)c4ccccc4C54c5ccccc5Sc5ccccc54)n3)c2)cc1]

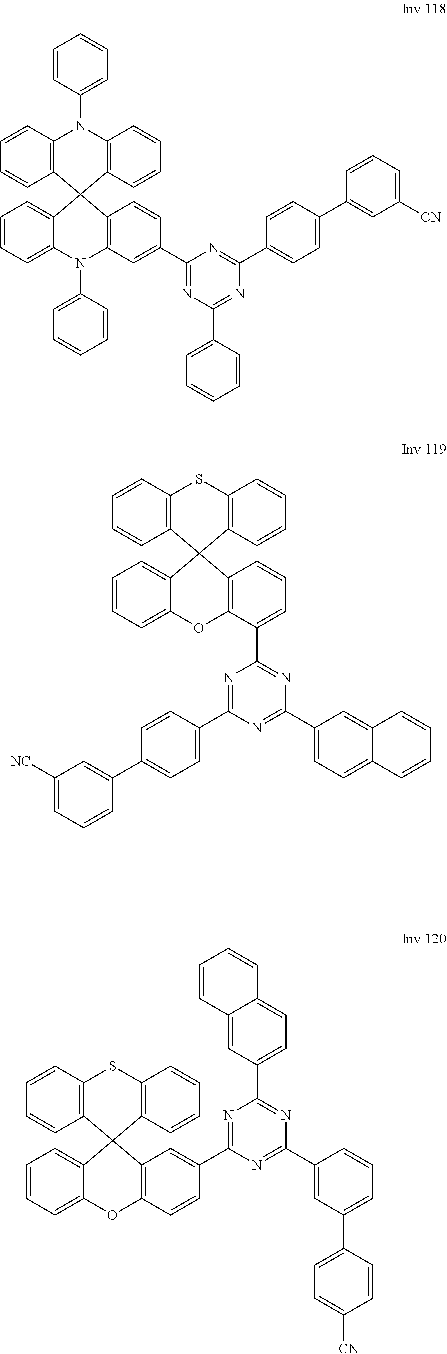 [SMILES: N#Cc1cccc(-c2ccc(-c3nc(-c4ccccc4)nc(-c4ccc5c(c4)N(c4ccccc4)c4ccccc4C54c5ccccc5N(c5ccccc5)c5ccccc54)n3)cc2)c1 ; N#Cc1cccc(-c2ccc(-c3nc(-c4ccc5ccccc5c4)nc(-c4cccc5c4Oc4ccccc4C54c5ccccc5Sc5ccccc54)n3)cc2)c1 ; N#Cc1ccc(-c2cccc(-c3nc(-c4ccc5c(c4)C4(c6ccccc6O5)c5ccccc5Sc5ccccc54)nc(-c4ccc5ccccc5c4)n3)c2)cc1]

As used herein, “the number of nuclear atoms” means the number of ring atoms constituting a ring structure, and the nuclear atoms may mean carbon or a heteroatom selected from the group consisting of N, O, S and Se. For example, the number of nuclear atoms of pyridine means 6 including 5 C and 1 N constituting a pyridine ring.

As used herein, “alkyl” refers to a monovalent substituent derived from a linear or branched chain saturated hydrocarbon having 1 to 40 carbon atoms. Examples of such alkyl may include, but not limited to, methyl, ethyl, propyl, isobutyl, sec-butyl, pentyl, iso-amyl, hexyl or the like.

As used herein, “alkenyl” refers to a monovalent substituent derived from a C₂ to C₄₀ linear or branched chain unsaturated hydrocarbon, having at least one carbon-carbon double bond. Examples of such alkenyl may include, but not limited to, vinyl, allyl, isopropenyl, 2-butenyl or the like.

As used herein, “alkynyl” refers to a monovalent substituent derived from a C₂ to C₄₀ linear or branched chain unsaturated hydrocarbon, having at least one carbon-carbon triple bond. Examples of such alkynyl may include, but not limited to, ethynyl, 2-propynyl or the like.

As used herein, “aryl” refers to a monovalent substituent derived from a C₆ to C₄₀ aromatic hydrocarbon having a structure with a single ring or two or more rings combined with each other. In addition, a form in which two or more rings are pendant (e.g., simply attached) to or fused with each other may also be included. Examples of such aryl may include, but not limited to, phenyl, naphthyl, phenanthryl, anthryl or the like.

As used herein, “heteroaryl” refers to a monovalent substituent derived from a monoheterocyclic or polyheterocyclic aromatic hydrocarbon having 5 to 40 nuclear atoms. In such an embodiment, one or more carbons in the ring, preferably one to three carbons, are substituted with a heteroatom such as N, O, S or Se. In addition, a form in which two or more rings are pendant to or fused with each other may be included and a form fused with an aryl group may be included. Examples of such heteroaryl may include, but not limited to, a 6-membered monocyclic ring such as pyridyl, pyrazinyl, pyrimidinyl, pyridazinyl and triazinyl; a polycyclic ring such as phenoxathienyl, indolizinyl, indolyl, purinyl, quinolyl, benzothiazole, and carbazolyl; 2-furanyl; N-imidazolyl; 2-isoxazolyl; 2-pyridinyl; 2-pyrimidinyl or the like.

As used herein, “aryloxy” is a monovalent substituent represented by RO—, where R refers to a C₅ to C₄₀ aryl. Examples of such aryloxy may include, but not limited to, phenyloxy, naphthyloxy, diphenyloxy or the like.

As used herein, “alkyloxy” refers to a monovalent substituent represented by R′O—, where R′ refers to a C₁ to C₄₀ alkyl. Such alkyloxy may include a linear, branched or cyclic structure. Examples of such alkyloxy may include, but not limited to, methoxy, ethoxy, n-propoxy, 1-propoxy, t-butoxy, n-butoxy, pentoxy or the like.

As used herein, “arylamine” refers to amine substituted with a C₆ to C₄₀ aryl.

As used herein, “cycloalkyl” refers to a monovalent substituent derived from a C₃ to C₄₀ monocyclic or polycyclic non-aromatic hydrocarbon. Examples of such cycloalkyl may include, but not limited to, cyclopropyl, cyclopentyl, cyclohexyl, norbornyl, adamantine or the like.

As used herein, “heterocycloalkyl” refers to a monovalent substituent derived from a non-aromatic hydrocarbon having 3 to 40 nuclear atoms, where one or more carbons in the ring, preferably one to three carbons, are substituted with a heteroatom such as N, O, S or Se. Examples of such heterocycloalkyl may include, but not limited to, morpholine, piperazine or the like.

As used herein, “alkylsilyl” refers to silyl substituted with a C₁ to C₄₀ alkyl, and “arylsilyl” refers to silyl substituted with a C₅ to C₄₀ aryl.

As used herein, the term “fused ring (e.g., condensed ring)” refers to a condensed aliphatic ring, a condensed aromatic ring, a condensed heteroaliphatic ring, a condensed heteroaromatic ring, or a combination thereof.

### <Electron Transport Layer Material>

The present invention provides an electron transport layer including the compound represented by Chemical Formula 1.

The electron transport layer (ETL) serves to move electrons injected from a cathode to an adjacent layer, specifically a light emitting layer.

The compound represented by Chemical Formula 1 may be used alone as an electron transport layer (ETL) material, or may be used in combination with an electron transport layer material known in the art. It may preferably be used alone.

The electron transport layer material that may be used in combination with the compound of Chemical Formula 1 may include an electron transport material commonly known in the art. Non-limiting examples of applicable electron transport materials may include oxazole-based compounds, isoxazole-based compounds, triazole-based compounds, isothiazole-based compounds, oxadiazole-based compounds, thiadiazole-based compounds, perylene-based compounds, aluminum complexes (e.g., tris(8-quinolinolato)-aluminium (Alq₃), BAlq, SAlq, Almq₃, gallium complexes (e.g., Gaq′2OPiv, Gaq′2OAc, 2(Gaq′2)), etc. These may be used alone or two or more types thereof may be used in combination.

In the present invention, when the compound of Chemical Formula 1 and the electron transport layer material are used in combination, a mixing ratio thereof is not particularly limited, and may be appropriately adjusted within a range known in the art.

### <Auxiliary Electron Transport Layer Material>

In addition, the present invention provides an auxiliary electron transport layer including the compound represented by Chemical Formula 1.

The auxiliary electron transport layer is disposed between the light emitting layer and the electron transport layer and serves to substantially prevent diffusion of excitons or holes generated in the light emitting layer into the electron transport layer.

The compound represented by Chemical Formula 1 may be used alone as an auxiliary electron transport layer material, or may be combined with an electron transport layer material known in the art. It may preferably be used alone.

The auxiliary electron transport layer material that may be used in combination with the compound of Chemical Formula 1 includes an electron transport material commonly known in the art. For example, the auxiliary electron transport layer may include an oxadiazole derivative, a triazole derivative, a phenanthroline derivative (e.g., BCP), a heterocyclic derivative containing nitrogen, and the like.

In the present invention, when the compound of Chemical Formula 1 and the auxiliary electron transport layer material are used in combination, a mixing ratio thereof is not particularly limited, and may be appropriately adjusted within a range known in the art.

### <Organic Electroluminescent Device>

Another aspect of the present invention is directed to an organic electroluminescent device (“organic EL device” or “organic EL element”) including the compound represented by Chemical Formula 1.

More specifically, the organic EL device according to the present invention includes an anode (e.g., a positive electrode), a cathode (e.g., a negative electrode), and one or more organic layers disposed between the anode and the cathode, and at least one of the one or more organic layers includes the compound represented by Chemical Formula 1. In such an embodiment, the compound may be used alone or in combination of two or more kinds thereof.

The one or more organic layers may be any one or more of a hole injection layer, a hole transport layer, a light emitting layer, a light emitting auxiliary layer, a lifespan improvement layer, an electron transport layer, an auxiliary electron transport layer and an electron injection layer, and at least one of the organic layers may include the compound represented by Chemical Formula 1. Specifically, the organic layer including the compound represented by Chemical Formula 1 may preferably be a light emitting layer, a light emitting auxiliary layer, an electron transport layer, an auxiliary electron transport layer and/or a lifespan improvement layer, and more specifically, it may be preferable to be an electron transport layer or an auxiliary electron transport layer.

The light emitting layer of the organic EL device of the present invention includes a host material and a dopant material, and may include the compound of Chemical Formula 1 as a host material. In addition, the light emitting layer of the present invention may include, as a host, other compounds known in the art rather than or in addition to the compound of Chemical Formula 1.

When the compound represented by Chemical Formula 1 is included as a material for the light emitting layer of the organic EL device, preferably as a blue, green, or red phosphorescent host material, since a bonding force between holes and electrons in the light emitting layer is increased, the efficiency (luminescence efficiency and power efficiency), lifespan, luminance, driving voltage, and the like of the organic EL device may be improved. Specifically, the compound represented by Chemical Formula 1 may be preferably included in an organic EL device as a green and/or red phosphorescent host, a fluorescent host, or a dopant material. In particular, the compound represented by Chemical Formula 1 of the present invention may preferably be a green phosphorescent exciplex N-type host material for a high-efficiency light emitting layer.

A structure of the organic EL device of the present invention is not particularly limited, and may be, for example, a structure in which a substrate, an anode, a hole injection layer, a hole transport layer, a light emitting auxiliary layer, a light emitting layer, an electron transport layer, and a cathode are sequentially stacked. In such a case, at least one of the hole injection layer, the hole transport layer, the light emitting auxiliary layer, the light emitting layer, the electron transport layer and the electron injection layer may include the compound represented by Chemical Formula 1, and preferably, the light emitting layer, more preferably a phosphorescent host, may include the compound represented by Chemical Formula 1. Meanwhile, an electron injection layer may be additionally stacked on the electron transport layer.

The organic EL device of the present invention may have a structure in which an insulating layer or an adhesive layer is inserted at an interface between the electrode and the organic layer.

The organic EL device of the present invention may be prepared using materials and methods known in the art to form organic layers and electrodes, except that one or more layers of the aforementioned organic layers include the compound represented by Chemical Formula 1.

The organic layer may be formed by a vacuum deposition method or a solution coating method. Examples of the solution coating method may include, but not limited to, spin coating, dip coating, doctor blading, inkjet printing, thermal transfer or the like.

The substrate used in preparation of the organic EL device of the present invention is not particularly limited, and non-limiting examples thereof may include silicon wafers, quartz, glass plates, metal plates, plastic films, sheets or the like.

In addition, an anode material may use any anode material known in the art without limitation. Examples of the anode material may include, but not limited to, a metal such as vanadium, chromium, copper, zinc, and gold or an alloy thereof; metal oxides such as zinc oxide, indium oxide, indium tin oxide (ITO), or indium zinc oxide (IZO); combination of oxide with metal such as ZnO:Al or SnO₂:Sb; conductive polymers such as polythiophene, poly(3-methylthiophene), poly[3,4-(ethylene-1,2-dioxy)thiophene](PEDT), polypyrrole or polyaniline; carbon black or the like.

In addition, a cathode material may use any cathode material known in the art without limitation. Examples of the cathode material may include, but not limited to, a metal such as magnesium, calcium, sodium, potassium, titanium, indium, yttrium, lithium, gadolinium, aluminum, silver, tin, or lead or an alloy thereof; a multi-layered material such as LiF/Al or LiO₂/Al or the like.

In addition, materials for the hole injection layer, the hole transport layer, the electron injection layer, and the electron transport layer are not particularly limited, and conventional materials known in the art may be used without limitation.

Hereinafter, the present invention will be described in detail through examples. However, the following examples are only to illustrate the present invention, and the present invention is not limited by the following examples.

### Preparation Examples 1 to 9: Synthesis of Core

The polycyclic spiro-based core structure according to the present invention may be prepared according to the following Reaction Scheme 1. However, the present invention is not limited thereto, and may be prepared according to a conventional reaction method known in the art.

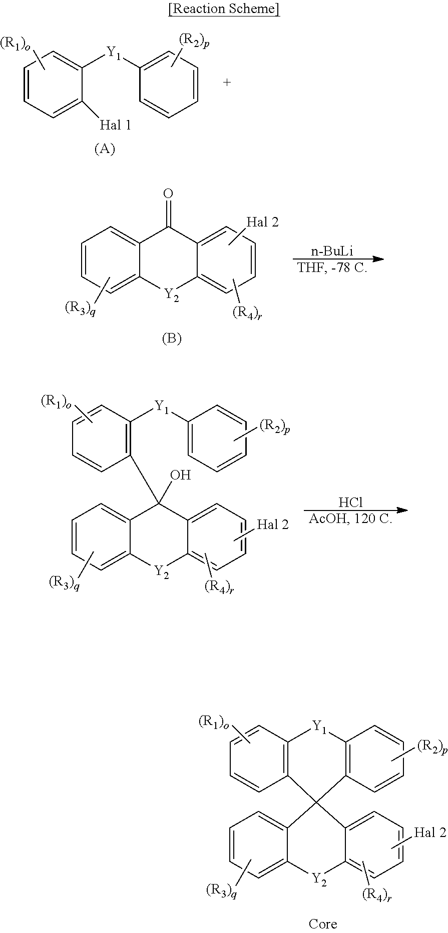

In the above Reaction Scheme, Hal 1 of the compound (A) may be a conventional halogen atom known in the art, and specifically, may be Br, I. In addition, Hal 2 of the compound (B) may be a conventional halogen atom, and specifically, may be Cl, Br, or I.

In the core structure, Y₁ to Y₂, R₁ to R₄, o, p, q, and r are each as defined in Chemical Formula 1.

### Preparation Example 1

### <Step 1> Synthesis of 2-chlorospiro[thioxanthene-9,9′-xanthene

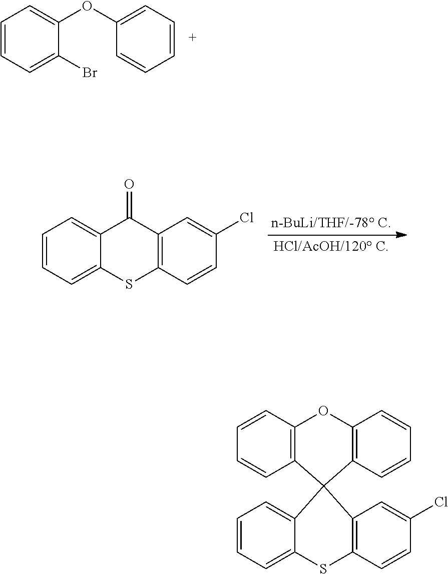 [SMILES: Brc1ccccc1Oc1ccccc1 ; O=c1c2ccccc2sc2ccc(Cl)cc12 ; Clc1ccc2c(c1)C1(c3ccccc3Oc3ccccc31)c1ccccc1S2]

1-bromo-2-phenoxybenzene (50 g, 200.7 mmol) as the compound (A) of Reaction Scheme 1 was dissolved in 670 ml of THF under a nitrogen atmosphere, and then a 1.6 M n-BuLi solution (134 ml, 220.8 mmol) was added thereto while stirring at −78° C., and then stirred for 1 hour. 2-chloro-9H-thioxanthen-9-one (54.4 g, 220.8 mmol) as the compound (B) of Reaction Scheme 1 was dissolved in 220 ml of THF and then added dropwise thereto. After stirring for 30 minutes, the temperature was raised to room temperature and stirred for 2 hours. After confirming the loss of the raw material, the solvent was concentrated under reduced pressure, 115 ml of AcOH and 115 ml of HCl were added thereto, and the resulting mixture was heated and stirred at 70° C. for 1 hour. The reaction solution was cooled to room temperature, and the reaction solution was extracted with dichloromethane, and MgSO₄ was added thereto to remove moisture, and then filtered. After the filtration, the solvent of the organic layer was concentrated under reduced pressure, and purified by column chromatography using dichloromethane and hexane, and then solidified using methanol. The solid was filtered, washed with methanol, and dried in an oven to obtain 2-chlorospirothioxanthene-9,9′-xanthene (45.0 g, yield 56.2%).

Mass [(M+H)⁺]: 399

### <Step 2> Synthesis of Core 1

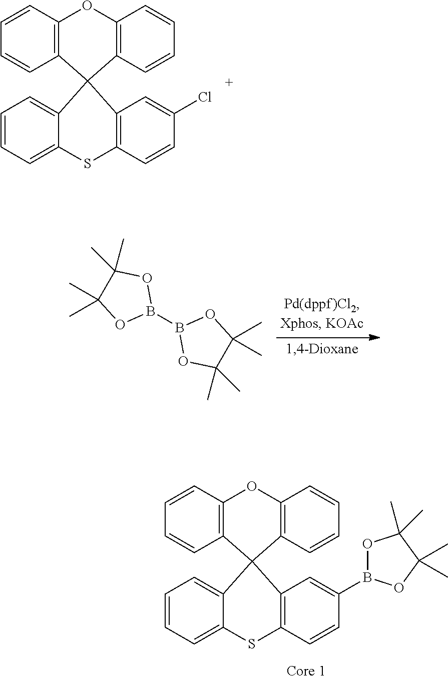

2-chlorospiro[thioxanthene-9,9′-xanthene](45.0 g, 112.8 mmol), 4,4,4′,4′,5,5,5′,5′-octamethyl-2,2′-bi(1,3,2-dioxaborolane) (37.2 g, 146.6 mmol), Pd(dppf)Cl₂ (2.5 g, 6.7 mmol), KOAc (65.3 g, 665.4 mmol), and Xphos (6.3 g, 13.3 mmol) were added to 750 ml of 1,4-Dioxane and heated to reflux for 12 hours. After the reaction was completed, the organic layer was extracted with dichloromethane, and MgSO₄ was added thereto to remove moisture, and then filtered. After the filtration, the solvent of the organic layer was concentrated under reduced pressure, and purified by column chromatography using dichloromethane and hexane, and then solidified using methanol. The solid was filtered, washed with methanol, and dried in an oven to obtain Core 1 (45.2 g, yield 81.7%).

Mass: [(M+H)⁺]: 491

### Preparation Example 2

### <Step 1> Synthesis of 4′-chlorospiro[thioxanthene-9,9′-xanthene]

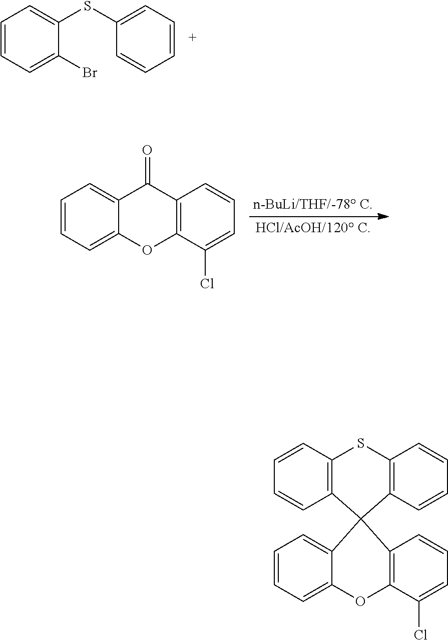 [SMILES: Brc1ccccc1Sc1ccccc1 ; O=c1c2ccccc2oc2c(Cl)cccc12 ; Clc1cccc2c1Oc1ccccc1C21c2ccccc2Sc2ccccc21]

In Step 1 of Preparation Example 1, (2-bromophenyl) (phenyl)sulfane (50 g, 188.6 mmol), 4-chloro-9H-xanthen-9-one (47.8 g, 207.4 mmol), n-BuLi 1.6M solution (129 ml, 207.4 mmol), 830 ml of THF, 108 ml of AcOH, and 108 ml of HCl were used to obtain 4′-chlorospiro[thioxanthene-9,9′-xanthene](42.0 g, yield 55.84%) by the same method as in Step 1 of Preparation Example 1.

Mass [(M+H)⁺]: 399

### <Step 2> Synthesis of Core 2

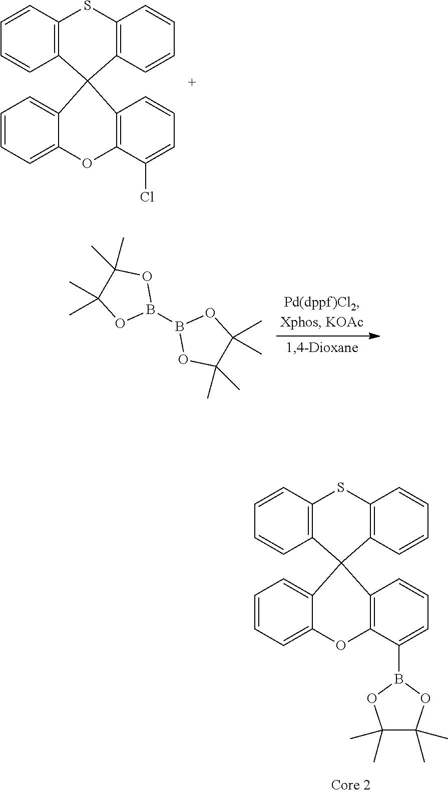

Except that 4′-chlorospiro[thioxanthene-9,9′-xanthene](42.0 g, 105.3 mmol) was used instead of 2-chlorospiro[thioxanthene-9,9′-xanthene] as reactant in Step 2 of Preparation Example 1, Core 2 (39.5 g, yield 76.5%) was obtained by performing the same procedure as in Step 2 of Preparation Example 1.

Mass: [(M+H)⁺]: 491

### Preparation Example 3

### <Step 1> Synthesis of 3-chloro-10-phenyl-10H-spiro[acridine-9,9′-xanthene

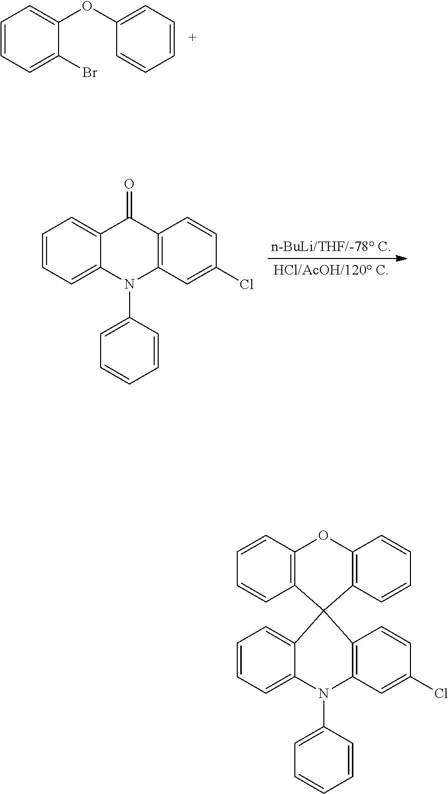 [SMILES: Brc1ccccc1Oc1ccccc1 ; O=c1c2ccccc2n(-c2ccccc2)c2cc(Cl)ccc12 ; Clc1ccc2c(c1)N(c1ccccc1)c1ccccc1C21c2ccccc2Oc2ccccc21]

In Step 1 of Preparation Example 1, 1-bromo-2-phenoxybenzene (50 g, 200.7 mmol), 3-chloro-10-phenylacridin-9(10H)-one (67.5 g, 220.8 mmol), n-BuLi 1.6M solution (138 ml, 220.8 mmol), 1000 ml of THF, 115 ml of AcOH 115, and 115 ml of HCl were used to obtain 3-chloro-10-phenyl-10H-spiro[acridine-9,9′-xanthene](48.1 g, yield 52.3%) by the same method as in Step 1 of Preparation Example 1.

Mass [(M+H)⁺]: 458

### <Step 2> Synthesis of Core 3

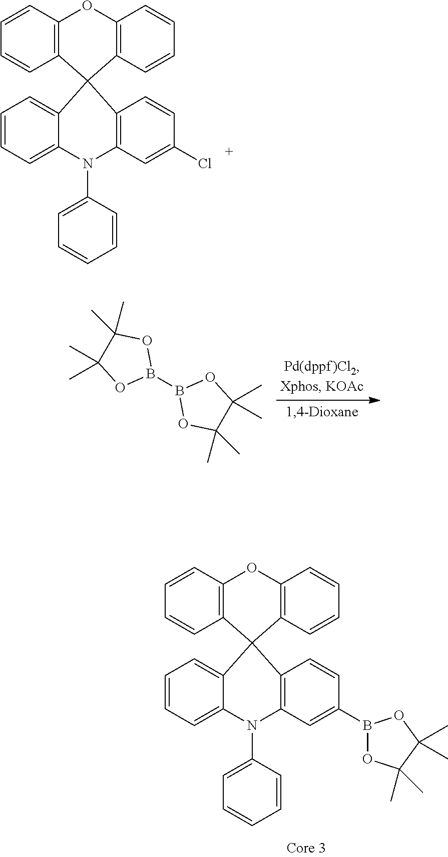

Except that 3-chloro-10-phenyl-10H-spiro[acridine-9,9′-xanthene](48.1 g, 105.0 mmol) was used instead of 2-chlorospiro[thioxanthene-9,9′-xanthene] as reactant in Step 2 of Preparation Example 1, Core 3 (35.5 g, yield 61.5%) was obtained by performing the same procedure as in Step 2 of Preparation Example 1.

Mass [(M+H)⁺]: 550

### Preparation Example 4

### <Step 1> Synthesis of 2′-chloro-10-phenyl-10H-spiro[acridine-9,9′-thioxanthene

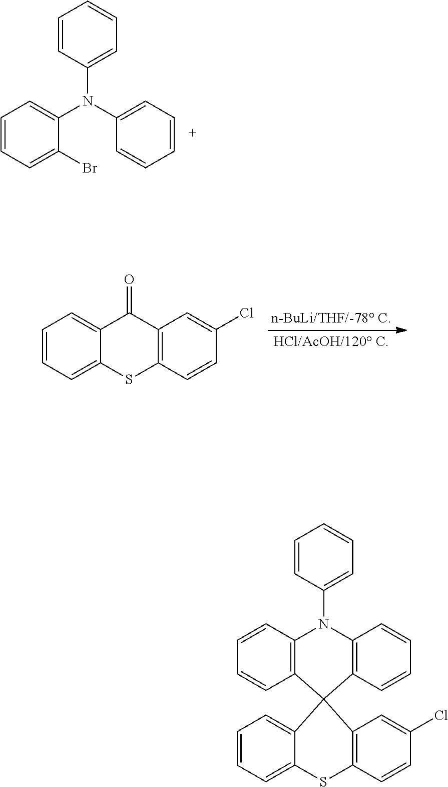 [SMILES: Brc1ccccc1N(c1ccccc1)c1ccccc1 ; O=c1c2ccccc2sc2ccc(Cl)cc12 ; Clc1ccc2c(c1)C1(c3ccccc3S2)c2ccccc2N(c2ccccc2)c2ccccc21]

In Step 1 of Preparation Example 1, 2-bromo-N,N-diphenylaniline (50 g, 154.2 mmol), 2-chloro-9H-thioxanthen-9-one (41.8 g, 169.6 mmol), n-BuLi 1.6M solution (106 ml, 169.6 mmol), 700 ml of THF, 89 ml of AcOH, and 89 ml of HCl were used to obtain 2′-chloro-10-phenyl-10H-spiro[acridine-9,9′-thioxanthene](37.8 g, yield 51.7%) by the same method as in Step 1 of Preparation Example 1.

Mass [(M+H)⁺]: 474

### <Step 2> Synthesis of Core 4

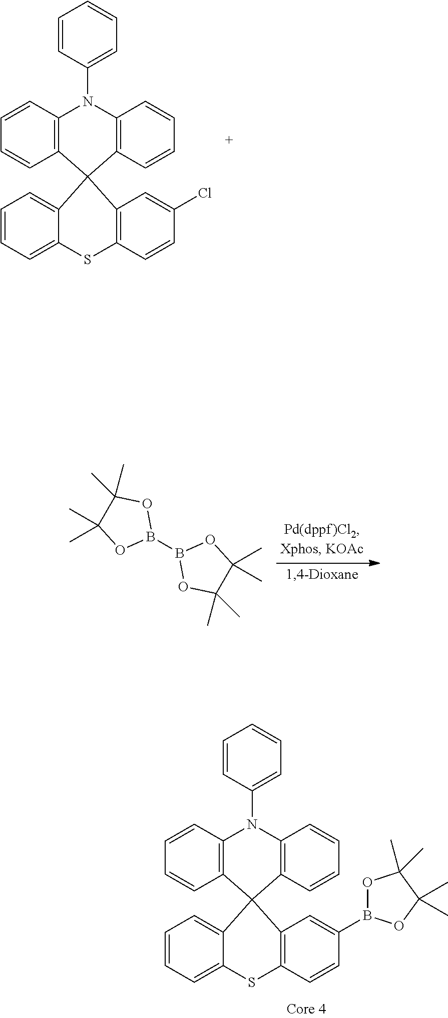

Except that 2′-chloro-10-phenyl-10H-spiro[acridine-9,9′-thioxanthene](37.8 g, 79.8 mmol) was used instead of 2-chlorospiro[thioxanthene-9,9′-xanthene] as reactant in Step 2 of Preparation Example 1, Core 4 (35.5 g, yield 78.7%) was obtained by performing the same procedure as in Step 2 of Preparation Example 1.

Mass [(M+H)⁺]: 566

### Preparation Example 5

### <Step 1> Synthesis of 2-chloro-10-phenyl-10H-spiro[acridine-9,9′-xanthene

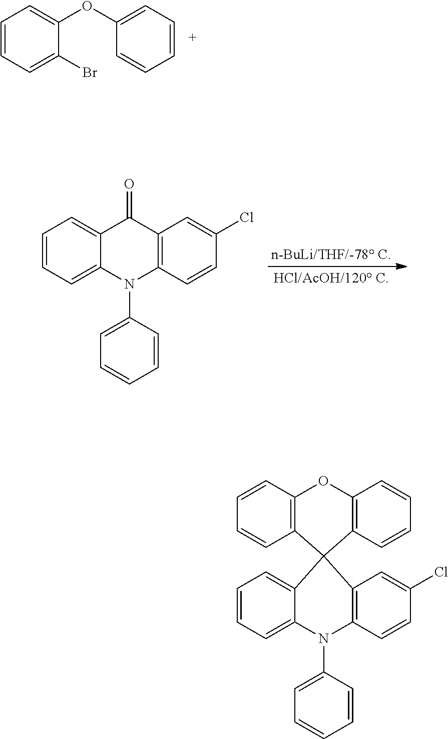 [SMILES: Brc1ccccc1Oc1ccccc1 ; O=c1c2ccccc2n(-c2ccccc2)c2ccc(Cl)cc12 ; Clc1ccc2c(c1)C1(c3ccccc3Oc3ccccc31)c1ccccc1N2c1ccccc1]

In Step 1 of Preparation Example 1, 1-bromo-2-phenoxybenzene (50 g, 200.7 mmol), 2-chloro-10-phenylacridin-9(10H)-one (67.5 g, 220.8 mmol), n-BuLi 1.6M solution (138 ml, 220.8 mmol), 900 ml of THF, 114 ml of AcOH, and 114 ml of HCl were used to obtain 2-chloro-10-phenyl-10H-spiro[acridine-9,9′-xanthene](60.0 g, yield 65.3%) by the same method as in Step 1 of Preparation Example 1.

Mass [(M+H)⁺]: 458

### <Step 2> Synthesis of Core 5

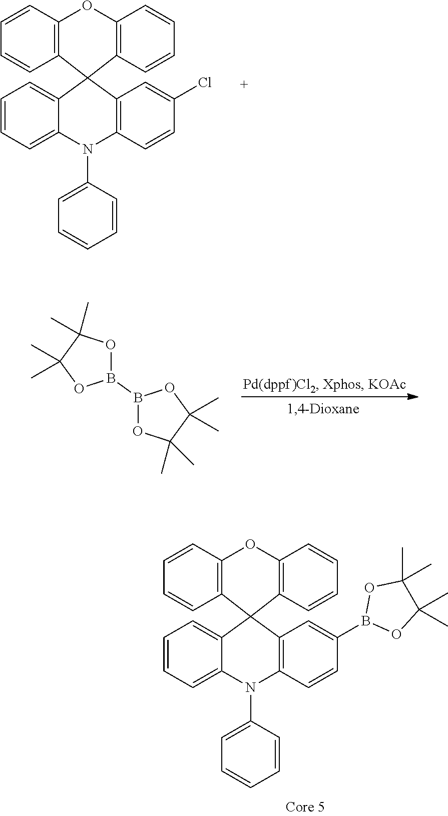

Except that 2-chloro-10-phenyl-10H-spiro[acridine-9,9′-xanthene](60.0 g, 131.0 mmol) was used instead of 2-chlorospiro[thioxanthene-9,9′-xanthene] as reactant in Step 2 of Preparation Example 1, Core 5 (48.8.g, yield 67.8%) was obtained by performing the same procedure as in Step 2 of Preparation Example 1.

Mass [(M+H)⁺]: 550

### Preparation Example 6

### <Step 1> Synthesis of 4′-chlorospiro[benzo[b]xanthene-12,9′-thioxanthene

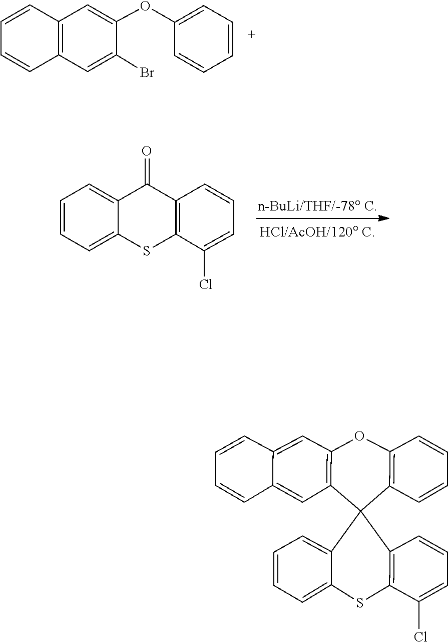 [SMILES: Brc1cc2ccccc2cc1Oc1ccccc1 ; O=c1c2ccccc2sc2c(Cl)cccc12 ; Clc1cccc2c1Sc1ccccc1C21c2ccccc2Oc2cc3ccccc3cc21]

In Step 1 of Preparation Example 1, 2-bromo-3-phenoxynaphthalene (30 g, 100.3 mmol), 4-chloro-9H-thioxanthen-9-one (27.2 g, 110.3 mmol), n-BuLi 1.6M solution (68.9 ml, 110.3 mmol), 450 ml of THF, 58 ml of AcOH, 58 ml of HCl were used to obtain 4′-chlorospiro[benzo[b]xanthene-12,9′-thioxanthene](24.6 g, yield 54.64%) by the same method as in Step 1 of Preparation Example 1.

Mass [(M+H)⁺]: 449

### <Step 2> Synthesis of Core 6

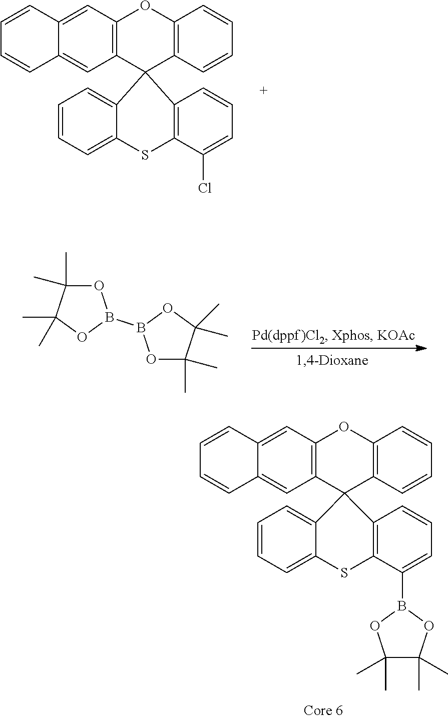

Except that 4′-chlorospiro[benzo[b]xanthene-12,9′-thioxanthene](24.6 g, 54.8 mmol) was used to instead of 2-chlorospiro[thioxanthene-9,9′-xanthene] as reactant in Step 2 of Preparation Example 1, Core 6 (20.0 g, yield 67.5%) was obtained by performing the same procedure as in Step 2 of Preparation Example 1.

Mass: [(M+H)⁺]: 540

### Preparation Example 7

<Step 1> Synthesis of 2-bromospiro[thioxanthene-9,9′-xanthene]-7-carbonitrile

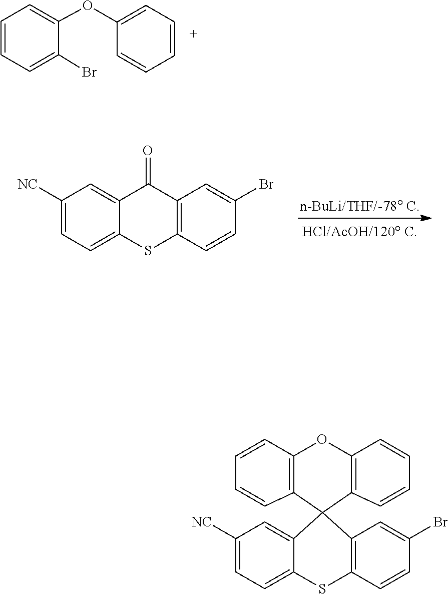 [SMILES: Brc1ccccc1Oc1ccccc1 ; N#Cc1ccc2sc3ccc(Br)cc3c(=O)c2c1 ; N#Cc1ccc2c(c1)C1(c3ccccc3Oc3ccccc31)c1cc(Br)ccc1S2]

In Step 1 of Preparation Example 1, 1-bromo-2-phenoxybenzene (30 g, 120.4 mmol), 7-bromo-9-oxo-9H-thioxanthene-2-carbonitrile (41.8 g, 132.5 mmol), n-BuLi 1.6M solution (83 ml, 132.5 mmol), 550 ml of THF, 69 ml of AcOH, and 69 ml of HCl were used to obtain 2-bromospiro[thioxanthene-9,9′-xanthene]-7-carbonitrile (26.2 g, yield 46.4%) by the same method as in Step 1 of Preparation Example 1.

Mass [(M+H)⁺]: 469

### <Step 2> Synthesis of Core 7

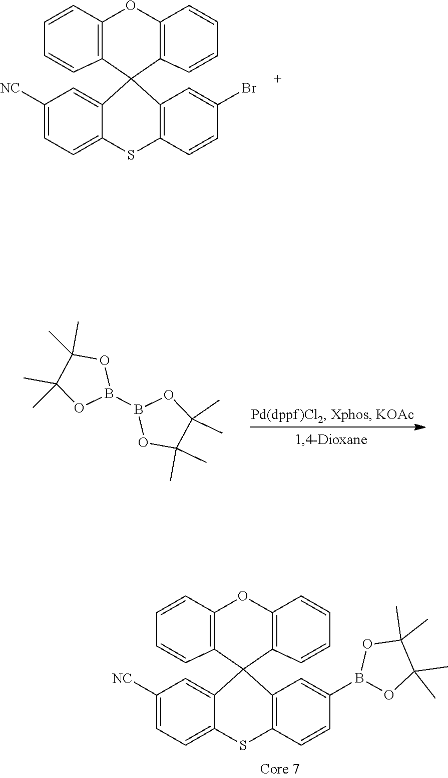

2-bromospiro[thioxanthene-9,9′-xanthene]-7-carbonitrile (26.2 g, 55.9 mmol), 4,4,4′,4′,5,5,5′,5′-octamethyl-2,2′-bi(1,3,2-dioxaborolane) (18.5 g, 72.7 mmol), Pd(dppf)Cl₂ (1.2 g, 1.7 mmol), and KOAc (16.5 g, 168 mmol) were added to 200 ml of 1,4-Dioxane and heated to reflux for 12 hours. After the reaction was completed, the reaction mixture was extracted with dichloromethane, MgSO₄ was added thereto to remove moisture, and then filtered. After the filtration, the solvent of the organic layer was concentrated under reduced pressure, purified by column chromatography using dichloromethane and hexane, and then solidified using methanol. The solid was filtered, washed with methanol, and dried in an oven to obtain Core 7 (20.5 g, yield 71.1%).

Mass [(M+H)⁺]515

### Preparation Example 8

### <Step 1> Synthesis of 4′-chloro-10-phenyl-10H-spiro[acridine-9,9′-thioxanthene

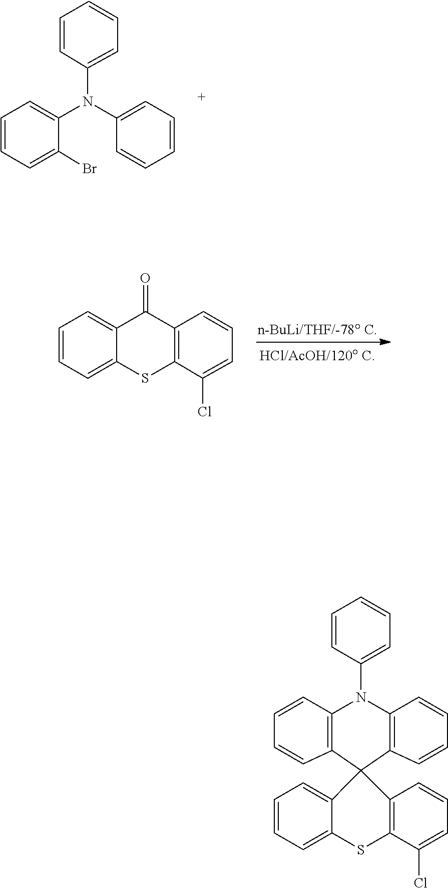 [SMILES: Brc1ccccc1N(c1ccccc1)c1ccccc1 ; O=c1c2ccccc2sc2c(Cl)cccc12 ; Clc1cccc2c1Sc1ccccc1C21c2ccccc2N(c2ccccc2)c2ccccc21]

In Step 1 of Preparation Example 1, 2-bromo-N,N-diphenylaniline (30 g, 92.5 mmol), 4-chloro-9H-thioxanthen-9-one (25.1 g, 101.8 mmol), n-BuLi 1.6M solution (63 ml, 101.8 mmol), 410 ml of THF, 53 ml of AcOH, 53 ml of HCl were used to obtain 4′-chloro-10-phenyl-10H-spiro[acridine-9,9′-thioxanthene](25.5 g, yield 58.1%) by the same method as in Step 1 of Preparation Example 1.

Mass: [(M+H)⁺]: 474

### <Step 2> Synthesis of Core 8

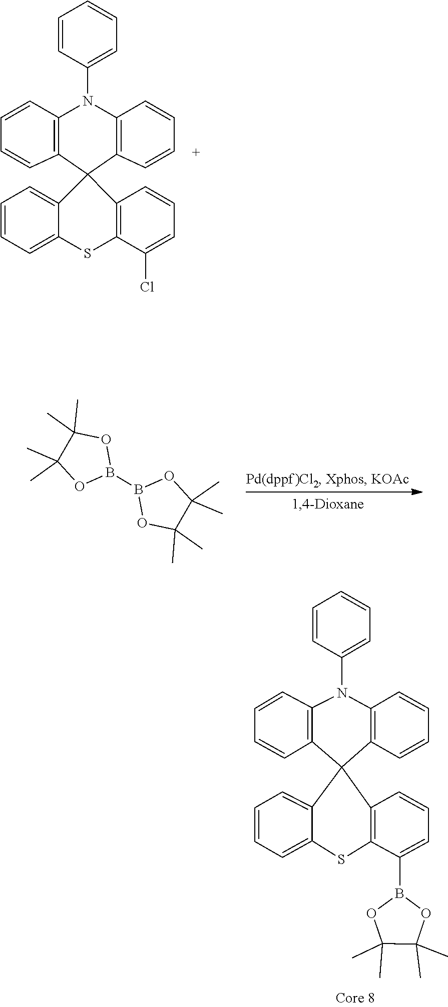

Except that 4′-chloro-10-phenyl-10H-spiro[acridine-9,9′-thioxanthene](25.5 g, 53.8 mmol) was used instead of 2-chlorospiro[thioxanthene-9,9′-xanthene] as reactant in Step 2 of Preparation Example 1, Core 8 (19.9 g, yield 65.4%) was obtained by performing the same procedure as in Step 2 of Preparation Example 1.

Mass [(M+H)⁺]: 565

### Preparation Example 9

### <Step 1> Synthesis of 4-bromo-10-phenyl-10H-spiro[acridine-9,9′-thioxanthene

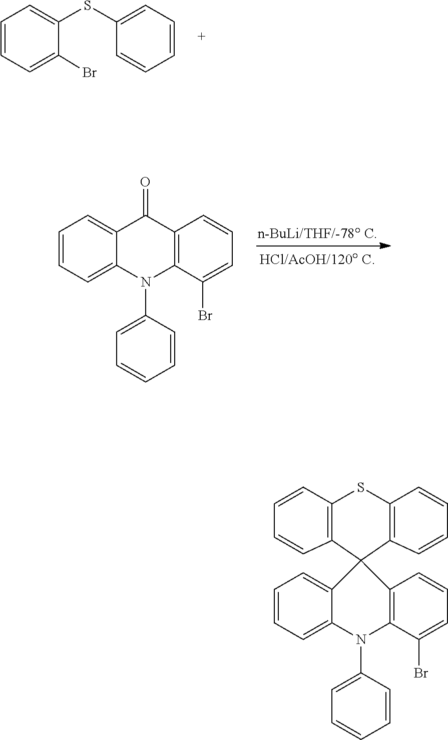 [SMILES: Brc1ccccc1Sc1ccccc1 ; O=c1c2ccccc2n(-c2ccccc2)c2c(Br)cccc12 ; Brc1cccc2c1N(c1ccccc1)c1ccccc1C21c2ccccc2Sc2ccccc21]

In Step 1 of Preparation Example 1, (2-bromophenyl) (phenyl)sulfane (50 g, 188.6 mmol), 4-bromo-10-phenylacridin-9(10H)-one (72.6 g, 207.4 mmol), n-BuLi 1.6M solution (129 ml, 207.4 mmol), 830 ml of THF, 108 ml of AcOH, and 108 ml of HCl were used to obtain 4-bromo-10-phenyl-10H-spiro[acridine-9,9′-thioxanthene](44.5 g, yield 59.2%) by the same method as as in Step 1 of Preparation Example 1.

Mass [(M+H)⁺]: 399

### <Step 2> Synthesis of Core 9

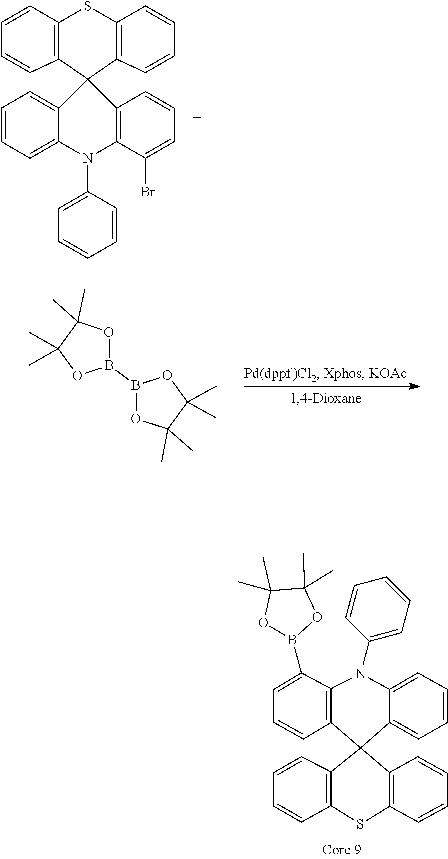

Except that 4-bromo-10-phenyl-10H-spiro[acridine-9,9′-thioxanthene](44.5 g, 111.5 mmol) was used instead of 2-bromospiro[thioxanthene-9,9′-xanthene]-7-carbonitrile as reactant in Step 2 of Preparation Example 7, Core 9 (38.0 g, yield 60.2%) was obtained by performing the same procedure as in Step 2 of Preparation Example 7.

Mass: [(M+H)⁺]: 566

### Synthesis Examples 1 to 19

### [Synthesis Example 1] Synthesis of Inv 6

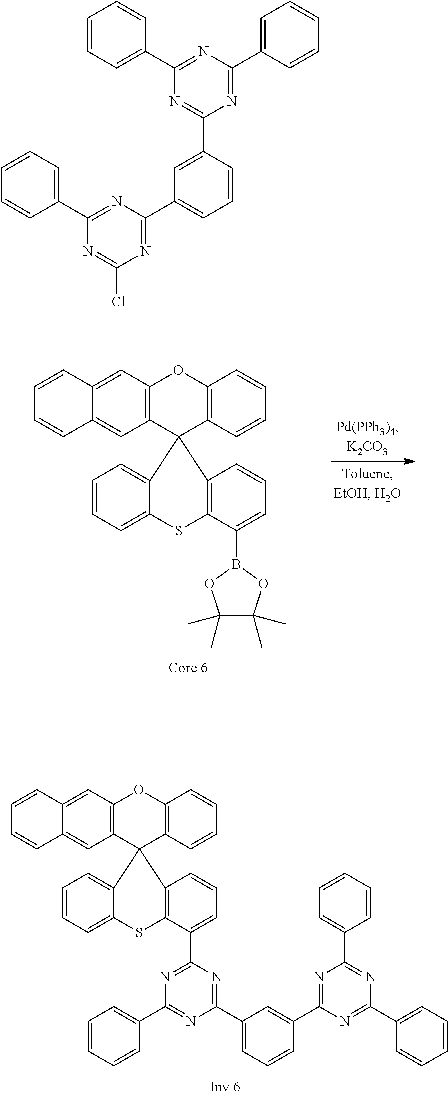 [SMILES: Clc1nc(-c2ccccc2)nc(-c2cccc(-c3nc(-c4ccccc4)nc(-c4ccccc4)n3)c2)n1 ; CC1(C)OB(c2cccc3c2Sc2ccccc2C32c3ccccc3Oc3cc4ccccc4cc32)OC1(C)C ; c1ccc(-c2nc(-c3ccccc3)nc(-c3cccc(-c4nc(-c5ccccc5)nc(-c5cccc6c5Sc5ccccc5C65c6ccccc6Oc6cc7ccccc7cc65)n4)c3)n2)cc1]

2-chloro-4-(3-(4,6-diphenyl-1,3,5-triazin-2-yl)phenyl)-6-phenyl-1,3,5-triazine (15.0 g, 30.1 mmol), Core 6 (17.9 g, 33.1 mmol) of the Preparation Example 6, Pd(Pph₃)₄(1.0 g, 0.9 mmol), and K₂CO₃ (12.5 g, 90.2 mmol) were added to 120 ml of Toluene, 30 ml of EtOH, 30 ml of H₂O, and the resulting mixture was heated to reflux for 4 hours. After the reaction was completed, the resulting mixture was extracted with dichloromethane, MgSO₄ was added thereto, and filtered. After removing the solvent of the filtered organic layer, the resulting mixture was subjected to column chromatography using dichloromethane and hexane, and then recrystallized with toluene acetone to obtain Inv 6 (28.2 g, yield 84.8%).

Mass [(M+H)⁺]: 878

### [Synthesis Example 2] Synthesis of Inv 7

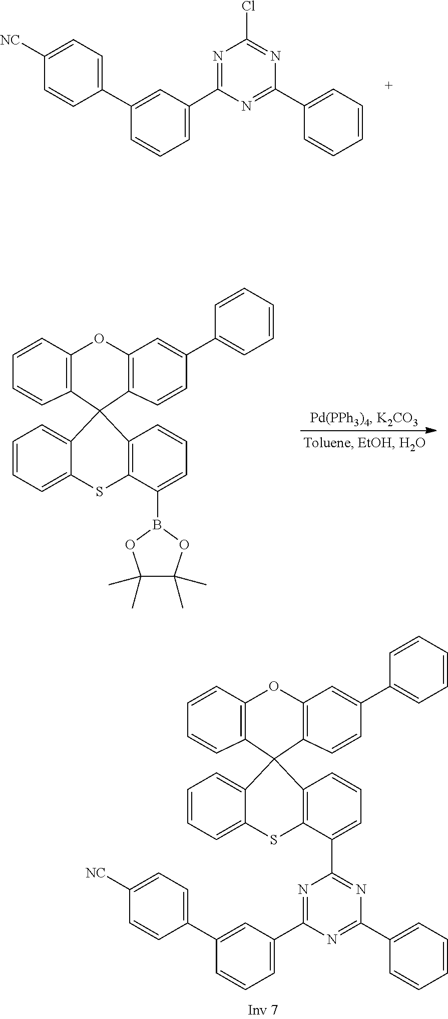 [SMILES: N#Cc1ccc(-c2cccc(-c3nc(Cl)nc(-c4ccccc4)n3)c2)cc1 ; CC1(C)OB(c2cccc3c2Sc2ccccc2C32c3ccccc3Oc3cc(-c4ccccc4)ccc32)OC1(C)C ; N#Cc1ccc(-c2cccc(-c3nc(-c4ccccc4)nc(-c4cccc5c4Sc4ccccc4C54c5ccccc5Oc5cc(-c6ccccc6)ccc54)n3)c2)cc1]

3′-(4-chloro-6-phenyl-1,3,5-triazin-2-yl)-[1,1′-biphenyl]-4-carbonitrile (15.0 g, 40.7 mmol), 4,4,5,5-tetramethyl-2-(3′-phenylspiro[thioxanthene-9,9′-xanthen]-4-yl)-1,3,2-dioxaborolane (25.3 g, 44.7 mmol), Pd(Pph₃)₄ (1.4 g, 1.2 mmol), and K₂CO₃ (16.9 g, 122.0 mmol) were added to 160 ml of Toluene, 40 ml of EtOH, and 40 ml of H₂O, and the resulting mixture was heated to reflux for 4 hours. After the reaction was completed, the resulting mixture was extracted with dichloromethane, MgSO₄ was added thereto, and filtered. After removing the solvent of the filtered organic layer, the resulting mixture was subjected to column chromatography using dichloromethane and hexane, and then recrystallized with toluene acetone to obtain Inv 7 (19.87 g, yield 62.9%).

Mass: [(M+H)⁺]: 773

### [Synthesis Example 3] Synthesis of Inv 8

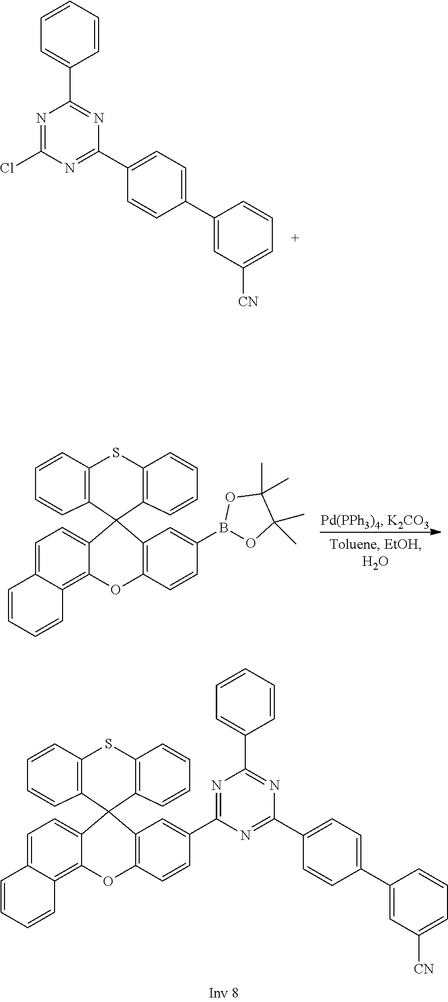 [SMILES: N#Cc1cccc(-c2ccc(-c3nc(Cl)nc(-c4ccccc4)n3)cc2)c1 ; CC1(C)OB(c2ccc3c(c2)C2(c4ccccc4Sc4ccccc42)c2ccc4ccccc4c2O3)OC1(C)C ; N#Cc1cccc(-c2ccc(-c3nc(-c4ccccc4)nc(-c4ccc5c(c4)C4(c6ccccc6Sc6ccccc64)c4ccc6ccccc6c4O5)n3)cc2)c1]

4′-(4-chloro-6-phenyl-1,3,5-triazin-2-yl)-[1,1′-biphenyl]-3-carbonitrile (15.0 g, 40.7 mmol), 4,4,5,5-tetramethyl-2-(spiro[benzo[c]xanthene-7,9′-thioxanthen]-9-yl)-1,3,2-dioxaborolane (24.2 g, 44.7 mmol), Pd(Pph₃)₄ (1.4 g, 1.2 mmol), and K₂CO₃ (16.9 g, 122.0 mmol) were added to 160 ml of Toluene, 40 ml of EtOH, 40 ml of H₂O, and the resulting mixture was heated to reflux for 4 hours. After the reaction was completed, the resulting mixture was extracted with dichloromethane, MgSO₄ was added thereto, and filtered. After removing the solvent of the filtered organic layer, the resulting mixture was subjected to column chromatography using dichloromethane and hexane, and then recrystallized with toluene acetone to obtain Inv 8 (21.0 g, yield 69.1%).

Mass: [(M+H)⁺]: 747

### [Synthesis Example 4] Synthesis of Inv 13

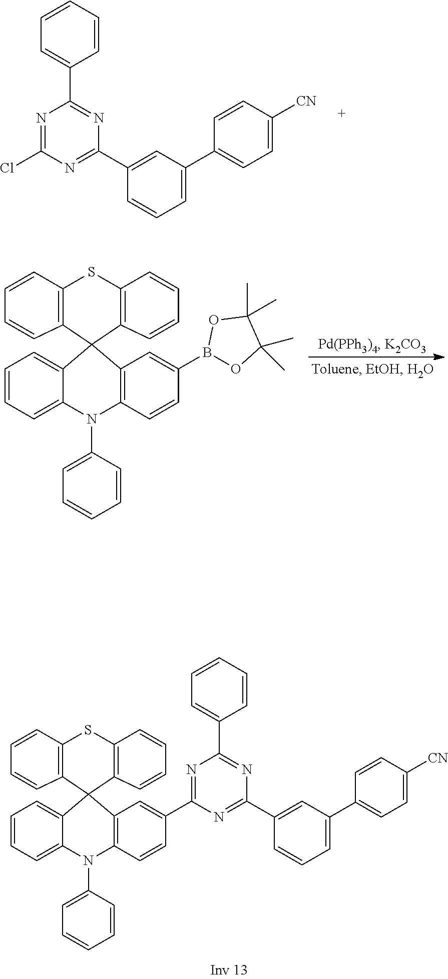 [SMILES: N#Cc1ccc(-c2cccc(-c3nc(Cl)nc(-c4ccccc4)n3)c2)cc1 ; CC1(C)OB(c2ccc3c(c2)C2(c4ccccc4Sc4ccccc42)c2ccccc2N3c2ccccc2)OC1(C)C ; N#Cc1ccc(-c2cccc(-c3nc(-c4ccccc4)nc(-c4ccc5c(c4)C4(c6ccccc6Sc6ccccc64)c4ccccc4N5c4ccccc4)n3)c2)cc1]

3′-(4-chloro-6-phenyl-1,3,5-triazin-2-yl)-[1,1′-biphenyl]-4-carbonitrile (15.0 g, 40.7 mmol), 10-phenyl-2-(4,4,5,5-tetramethyl-1,3,2-dioxaborolan-2-yl)-10H-spiro[acridine-9,9′-thioxanthene](25.3 g, 44.7 mmol), Pd(Pph₃)₄ (1.4 g, 1.2 mmol), and K₂CO₃ (16.9 g, 122.0 mmol) were added to 160 ml of Toluene, 40 ml of EtOH, and 40 ml of H₂O, and the resulting mixture was heated to reflux for 4 hours. After the reaction was completed, the resulting mixture was extracted with dichloromethane, MgSO₄ was added thereto, and filtered. After removing the solvent of the filtered organic layer, the resulting mixture was subjected to column chromatography using dichloromethane and hexane, and then recrystallized with toluene acetone to obtain Inv 13 (18.1 g, yield 57.7%).

Mass [(M+H)⁺]772

### [Synthesis Example 5] Synthesis of Inv 29

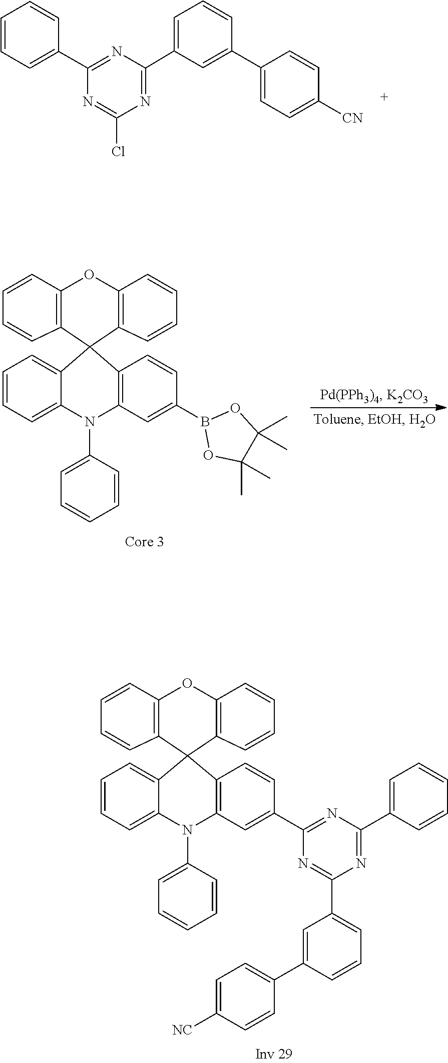 [SMILES: N#Cc1ccc(-c2cccc(-c3nc(Cl)nc(-c4ccccc4)n3)c2)cc1 ; CC1(C)OB(c2ccc3c(c2)N(c2ccccc2)c2ccccc2C32c3ccccc3Oc3ccccc32)OC1(C)C ; N#Cc1ccc(-c2cccc(-c3nc(-c4ccccc4)nc(-c4ccc5c(c4)N(c4ccccc4)c4ccccc4C54c5ccccc5Oc5ccccc54)n3)c2)cc1]

3′-(4-chloro-6-phenyl-1,3,5-triazin-2-yl)-[1,1′-biphenyl]-4-carbonitrile (15.0 g, 40.8 mmol), Core 3 (17.9 g, 33.1 mmol) of Preparation Example 3, Pd(Pph₃)₄ (1.4 g, 1.2 mmol), and K₂CO₃ (16.9 g, 122.3 mmol) were added to 160 ml of Toluene, 40 ml of EtOH, and 40 ml of H₂O, and the resulting mixture was heated to reflux for 4 hours. After the reaction was completed, the resulting mixture was extracted with dichloromethane, MgSO₄ was added thereto, and filtered. After removing the solvent of the filtered organic layer, the resulting mixture was subjected to column chromatography using dichloromethane and hexane, and then recrystallized with toluene acetone to obtain Inv 29 (24.6 g, yield 79.9%).

Mass [(M+H)⁺]: 755

### [Synthesis Example 6] Synthesis of Inv 40

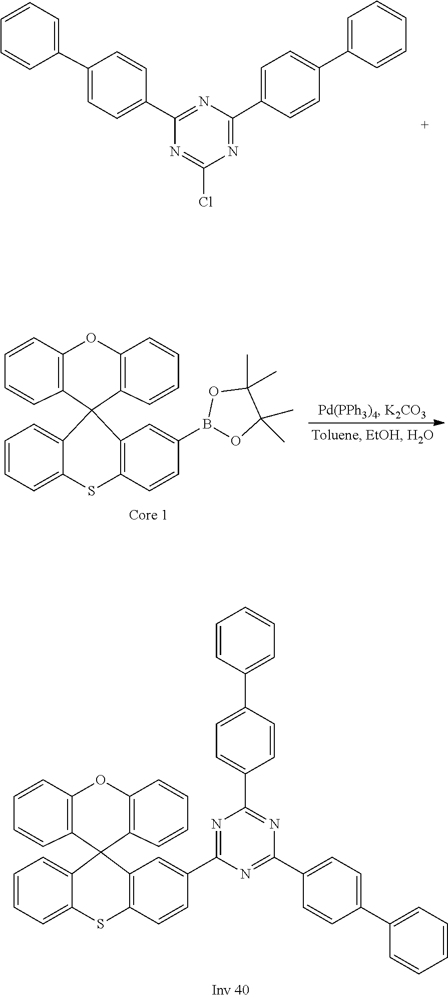 [SMILES: Clc1nc(-c2ccc(-c3ccccc3)cc2)nc(-c2ccc(-c3ccccc3)cc2)n1 ; CC1(C)OB(c2ccc3c(c2)C2(c4ccccc4Oc4ccccc42)c2ccccc2S3)OC1(C)C ; c1ccc(-c2ccc(-c3nc(-c4ccc(-c5ccccc5)cc4)nc(-c4ccc5c(c4)C4(c6ccccc6Oc6ccccc64)c4ccccc4S5)n3)cc2)cc1]

3′-(6-chloro-2-phenylpyrimidin-4-yl)-[1,1′-biphenyl]-4-carbonitrile (15.0 g, 35.7 mmol), Core 1 (19.3 g, 39.3 mmol) of Preparation Example 1, Pd(Pph₃)₄ (1.2 g, 1.07 mmol), and K₂CO₃ (14.8 g, 107.2 mmol) were added to 120 ml of Toluene, 30 ml of EtOH, and 30 ml of H₂O, and the resulting mixture was heated to reflux for 4 hours. After the reaction was completed, the resulting mixture was extracted with dichloromethane, MgSO₄ was added thereto, and filtered. After removing the solvent of the filtered organic layer, the resulting mixture was subjected to column chromatography using dichloromethane and hexane, and then recrystallized with toluene acetone to obtain Inv 40 (19.5 g, yield 72.9%).

Mass [(M+H)⁺]: 748

### [Synthesis Example 7] Synthesis of Inv 41

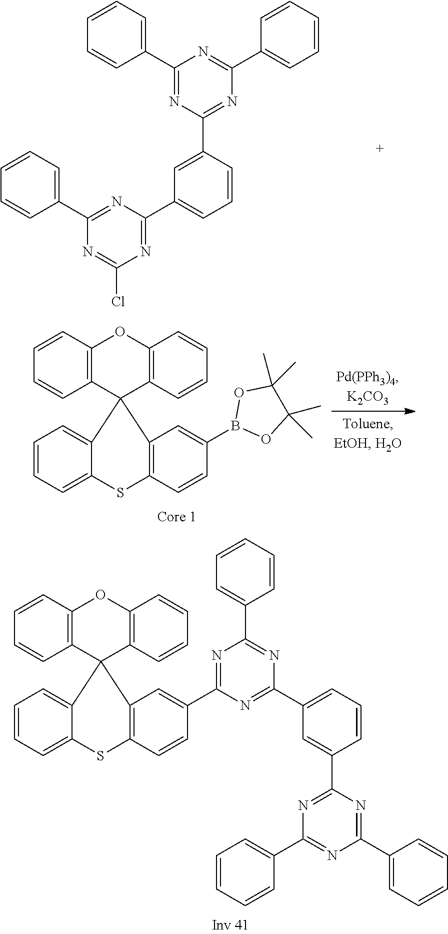

Except that 2-chloro-4-(3-(4,6-diphenyl-1,3,5-triazin-2-yl)phenyl)-6-phenyl-1,3,5-triazine (15.0 g 30.06 mmol) was used instead of 3′-(6-chloro-2-phenylpyrimidin-4-yl)-[1,1′-biphenyl]-4-carbonitrile, Inv 41 (15.8 g, yield 63.5%) was obtained by performing the same procedure as in Synthesis Example 3.

Mass [(M+H)⁺]: 827

### [Synthesis Example 8] Synthesis of Inv 44

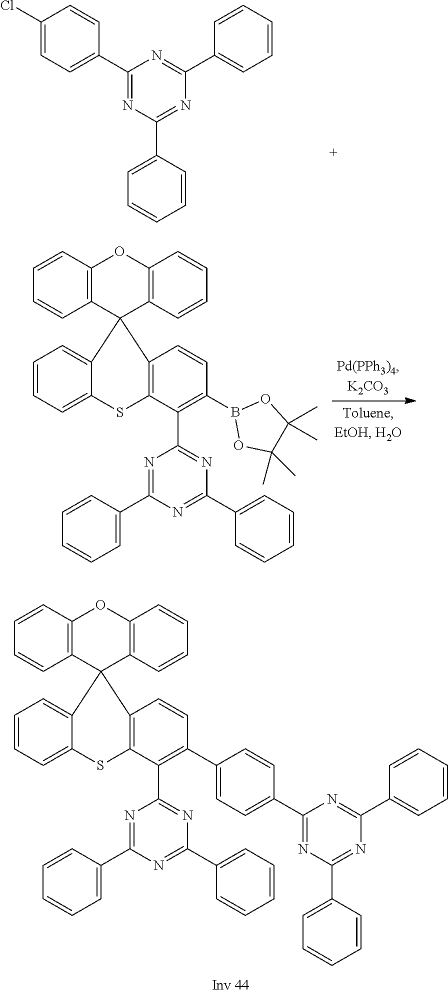

2-(4-chlorophenyl)-4,6-diphenyl-1,3,5-triazine (8.0 g, 23.3 mmol), 2,4-diphenyl-6-(3-(4,4,5,5-tetramethyl-1,3,2-dioxaborolan-2-yl)spiro[thioxanthene-9,9′-xanthen]-4-yl)-1,3,5-triazine (18.5 g, 25.6 mmol), Pd(OAc)₂ (0.2 g, 0.7 mmol), XPhos (1.1 g, 2.33 mmol), and Cs₂CO₃ (22.7 g, 69.8 mmol) were added to 100 ml of Toluene, 25 ml of EtOH, and 25 ml of H₂O, and the resulting mixture was heated to reflux for 4 hours. After the reaction was completed, the resulting mixture was extracted with dichloromethane, MgSO₄ was added thereto, and filtered. After removing the solvent of the filtered organic layer, the resulting mixture was subjected to column chromatography using dichloromethane and hexane, and then recrystallized with toluene acetone to obtain Inv 44 (16.5 g, yield 78.5%).

Mass: [(M+H)⁺]: 903

### [Synthesis Example 9] Synthesis of Inv 46

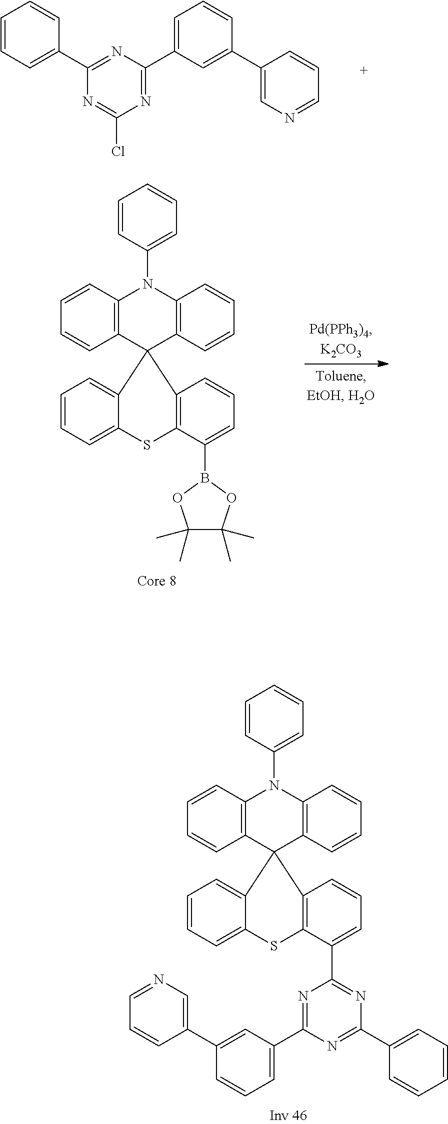

2-chloro-4-phenyl-6-(3-(pyridin-3-yl)phenyl)-1,3,5-triazine (15.0 g, 43.5 mmol), Core 8 (27.1 g, 47.8 mmol) of Preparation Example 8, Pd(Pph₃)₄ (1.5 g, 1.3 mmol), and K₂CO₃ (18.0 g, 130.5 mmol) were added to 160 ml of Toluene, 40 ml of EtOH, and 40 ml of H₂O, and the resulting mixture was heated to reflux for 4 hours. After the reaction was completed, the resulting mixture was extracted with dichloromethane, MgSO₄ was added thereto, and filtered. After removing the solvent of the filtered organic layer, the resulting mixture was subjected to column chromatography using dichloromethane and hexane, and then recrystallized with toluene acetone to obtain Inv 46 (28.0 g, yield 86.1%).

Mass [(M+H)⁺]: 748

### [Synthesis Example 10] Synthesis of Inv 54

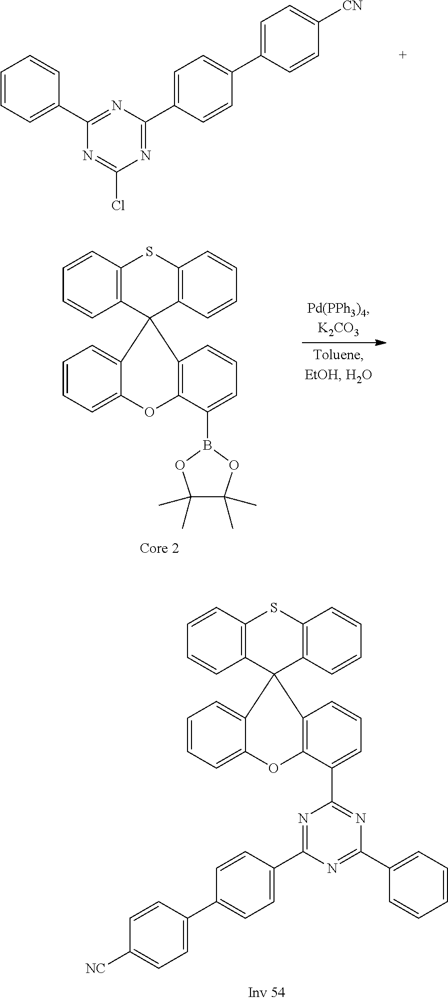 [SMILES: N#Cc1ccc(-c2ccc(-c3nc(Cl)nc(-c4ccccc4)n3)cc2)cc1 ; CC1(C)OB(c2cccc3c2Oc2ccccc2C32c3ccccc3Sc3ccccc32)OC1(C)C ; N#Cc1ccc(-c2ccc(-c3nc(-c4ccccc4)nc(-c4cccc5c4Oc4ccccc4C54c5ccccc5Sc5ccccc54)n3)cc2)cc1]

4′-(4-chloro-6-phenyl-1,3,5-triazin-2-yl)-[1,1′-biphenyl]-4-carbonitrile (15.0 g, 40.7 mmol), Core 2 (21.9 g, 44.7 mmol) of Preparation Example 2, Pd(Pph₃)₄ (1.4 g, 1.2 mmol), and K₂CO₃ (16.9 g, 122.0 mmol) were added to 160 ml of Toluene, 40 ml of EtOH, and 40 ml of H₂O, and the resulting mixture was heated to reflux for 4 hours. After the reaction was completed, the resulting mixture was extracted with dichloromethane, MgSO₄ was added thereto, and filtered. After removing the solvent of the filtered organic layer, the resulting mixture was subjected to column chromatography using dichloromethane and hexane, and then recrystallized with toluene acetone to obtain Inv 54 (22.5 g, yield 79.4%).

Mass [(M+H)⁺]: 697

### [Synthesis Example 11] Synthesis of Inv 56

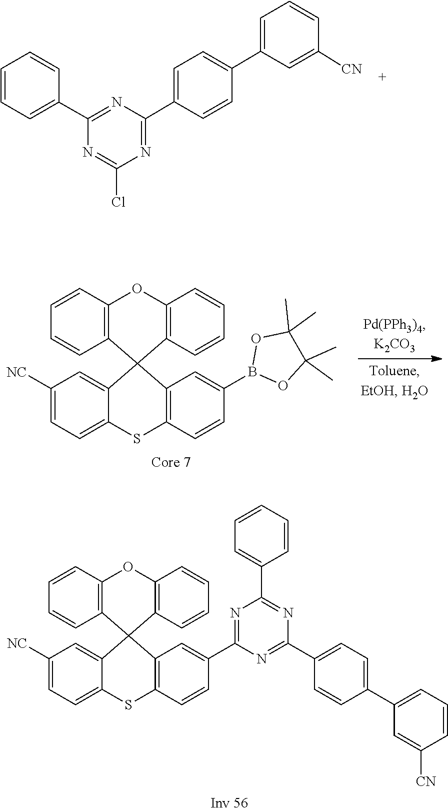 [SMILES: N#Cc1cccc(-c2ccc(-c3nc(Cl)nc(-c4ccccc4)n3)cc2)c1 ; CC1(C)OB(c2ccc3c(c2)C2(c4ccccc4Oc4ccccc42)c2cc(C#N)ccc2S3)OC1(C)C ; N#Cc1cccc(-c2ccc(-c3nc(-c4ccccc4)nc(-c4ccc5c(c4)C4(c6ccccc6Oc6ccccc64)c4cc(C#N)ccc4S5)n3)cc2)c1]

4′-(4-chloro-6-phenyl-1,3,5-triazin-2-yl)-[1,1′-biphenyl]-3-carbonitrile (15.0 g, 40.7 mmol), Core 7 (23.1 g, 44.7 mmol) of Preparation Example 7, Pd(Pph₃)₄ (1.4 g, 1.2 mmol), and K₂CO₃ (16.9 g, 122.0 mmol) were added to 160 ml of Toluene, 40 ml of EtOH, 40 ml of H₂O, and the resulting mixture was heated to reflux for 4 hours. After the reaction was completed, the resulting mixture was extracted with dichloromethane, MgSO₄ was added thereto, and filtered. After removing the solvent of the filtered organic layer, the resulting mixture was subjected to column chromatography using dichloromethane and hexane, and then recrystallized with toluene acetone to obtain Inv 56 (23.2 g, yield 79.0%).

Mass [(M+H)⁺]: 722

### [Synthesis Example 12] Synthesis of Inv 63

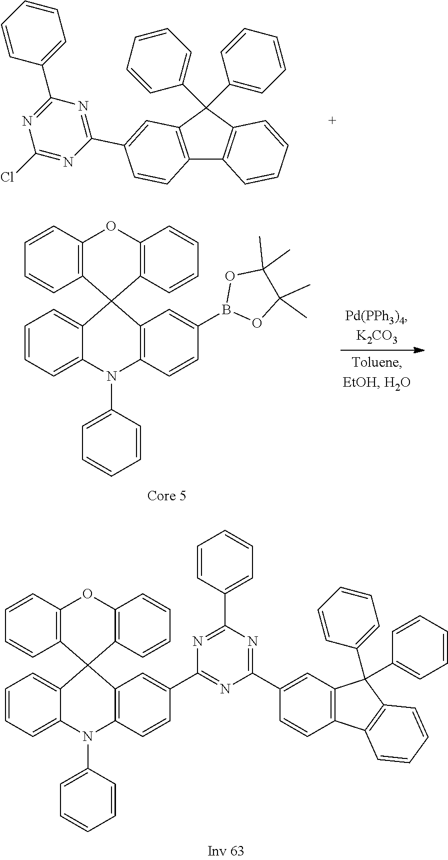

2-chloro-4-(9,9-diphenyl-9H-fluoren-2-yl)-6-phenyl-1,3,5-triazine (14.3 g, 28.2 mmol), Core 5 (17.0 g, 31.0 mmol) of Preparation Example 5, Pd(Pph₃)₄ (1.0 g, 0.85 mmol), and K₂CO₃ (11.7 g, 84.6 mmol) were added to 120 ml of Toluene, 30 ml of EtOH, and 30 ml of H₂O,

and the resulting mixture was heated to reflux for 4 hours. After the reaction was completed, the resulting mixture was extracted with dichloromethane, MgSO₄ was added thereto, and filtered. After removing the solvent of the filtered organic layer, the resulting mixture was subjected to column chromatography using dichloromethane and hexane, and then recrystallized with toluene acetone to obtain Inv 63 (21.0 g, yield 81.0%).

Mass [(M+H)⁺]: 920

### [Synthesis Example 13] Synthesis of Inv 78

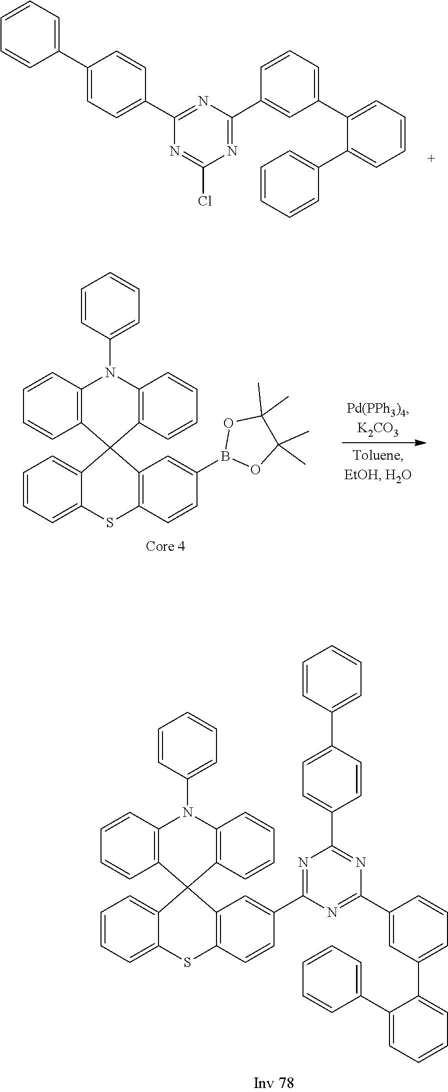 [SMILES: Clc1nc(-c2ccc(-c3ccccc3)cc2)nc(-c2cccc(-c3ccccc3-c3ccccc3)c2)n1 ; CC1(C)OB(c2ccc3c(c2)C2(c4ccccc4S3)c3ccccc3N(c3ccccc3)c3ccccc32)OC1(C)C ; c1ccc(-c2ccc(-c3nc(-c4cccc(-c5ccccc5-c5ccccc5)c4)nc(-c4ccc5c(c4)C4(c6ccccc6S5)c5ccccc5N(c5ccccc5)c5ccccc54)n3)cc2)cc1]

2-([1,1′-biphenyl]-4-yl)-4-([1,1′:2′,1″-terphenyl]-3-yl)-6-chloro-1,3,5-triazine (10.0 g, 20.2 mmol), Core 4 (12.5 g, 22.2 mmol) of Preparation Example 4, Pd(Pph₃)₄ (0.7 g, 0.6 mmol), and K₂CO₃ (8.4 g, 60.5 mmol) were added to 80 ml of Toluene, 20 ml of EtOH, and 20 ml of H₂O, and the resulting mixture was heated to reflux for 4 hours. After the reaction was completed, the resulting mixture was extracted with dichloromethane, MgSO₄ was added thereto, and filtered. After removing the solvent of the filtered organic layer, the resulting mixture was subjected to column chromatography using dichloromethane and hexane, and then recrystallized with toluene acetone to obtain Inv 78 (14.2 g, yield 78.3%).

Mass: [(M+H)⁺]: 899

### [Synthesis Example 14] Synthesis of Inv 86

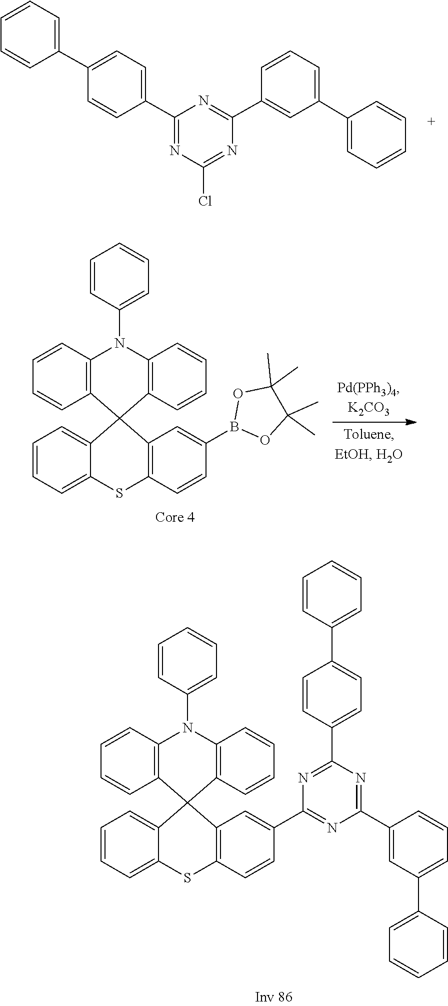 [SMILES: Clc1nc(-c2ccc(-c3ccccc3)cc2)nc(-c2cccc(-c3ccccc3)c2)n1 ; CC1(C)OB(c2ccc3c(c2)C2(c4ccccc4S3)c3ccccc3N(c3ccccc3)c3ccccc32)OC1(C)C ; c1ccc(-c2ccc(-c3nc(-c4cccc(-c5ccccc5)c4)nc(-c4ccc5c(c4)C4(c6ccccc6S5)c5ccccc5N(c5ccccc5)c5ccccc54)n3)cc2)cc1]

Except that 2-([1,1′-biphenyl]-3-yl)-4-([1,1′-biphenyl]-4-yl)-6-chloro-1,3,5-triazine (10.0 g, 23.8 mmol) was used instead of 2-([1,1′-biphenyl]-4-yl)-4-([1,1′:2′,1″-terphenyl]-3-yl)-6-chloro-1,3,5-triazine, Inv 86 (13.5 g, yield 68.9%) was obtained by performing the same procedure as in Synthesis Example 9.

Mass [(M+H)⁺]: 823

### [Synthesis Example 15] Synthesis of Inv 97

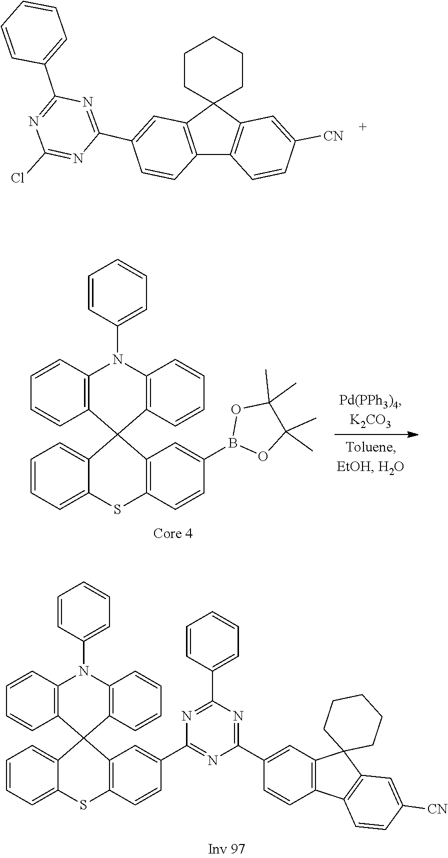 [SMILES: N#Cc1ccc2c(c1)C1(CCCCC1)c1cc(-c3nc(Cl)nc(-c4ccccc4)n3)ccc1-2 ; CC1(C)OB(c2ccc3c(c2)C2(c4ccccc4S3)c3ccccc3N(c3ccccc3)c3ccccc32)OC1(C)C ; N#Cc1ccc2c(c1)C1(CCCCC1)c1cc(-c3nc(-c4ccccc4)nc(-c4ccc5c(c4)C4(c6ccccc6S5)c5ccccc5N(c5ccccc5)c5ccccc54)n3)ccc1-2]

Except that 2′-(4-chloro-6-phenyl-1,3,5-triazin-2-yl)spiro[cyclohexane-1,9′-fluorene]-7′-carbonitrile (10.0 g, 22.33 mmol) was used instead of 2-([1,1′-biphenyl]-4-yl)-4-([1,1′:2′,1″-terphenyl]-3-yl)-6-chloro-1,3,5-triazine, Inv 97 (16.4 g, yield 86.4%) was obtained by performing the same procedure as in Preparation Example 9.

Mass: [(M+H)⁺]: 853

### [Synthesis Example 16] Synthesis of Inv 100

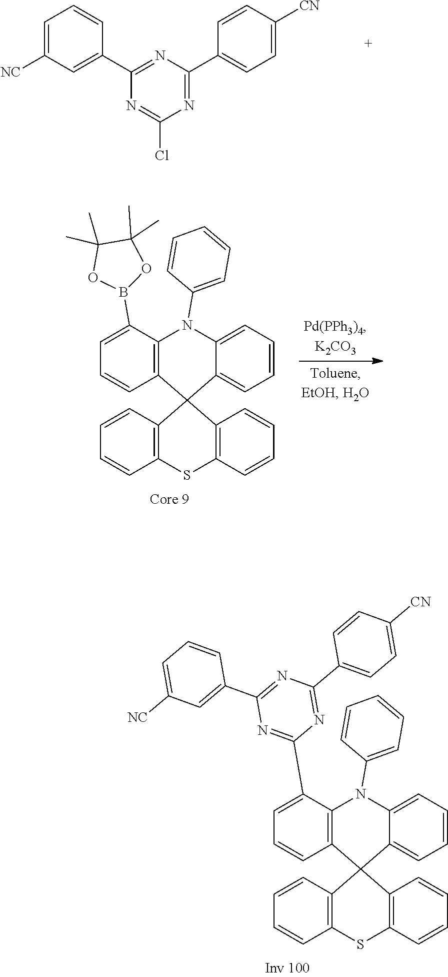 [SMILES: N#Cc1ccc(-c2nc(Cl)nc(-c3cccc(C#N)c3)n2)cc1 ; CC1(C)OB(c2cccc3c2N(c2ccccc2)c2ccccc2C32c3ccccc3Sc3ccccc32)OC1(C)C ; N#Cc1ccc(-c2nc(-c3cccc(C#N)c3)nc(-c3cccc4c3N(c3ccccc3)c3ccccc3C43c4ccccc4Sc4ccccc43)n2)cc1]

3-(4-chloro-6-(4-cyanophenyl)-1,3,5-triazin-2-yl)benzonitrile (15.0 g, 47.2 mmol), Core 9 (29.4 g, 51.9 mmol) of Preparation Example 9, Pd (Pph₃)₄ (1.6 g, 1.4 mmol), and K₂CO₃ (19.6 g, 141.6 mmol) were added to 160 ml of Toluene, 40 ml of EtOH, and 40 ml of H₂O, and the resulting mixture was heated to reflux for 4 hours. After the reaction was completed, the resulting mixture was extracted with dichloromethane, MgSO₄ was added thereto, and filtered. After removing the solvent of the filtered organic layer, the resulting mixture was subjected to column chromatography using dichloromethane and hexane, and then recrystallized with toluene acetone to obtain Inv 100 (28.8 g, yield 84.6%).

Mass: [(M+H)⁺]: 721

### [Synthesis Example 17] Synthesis of Inv 111

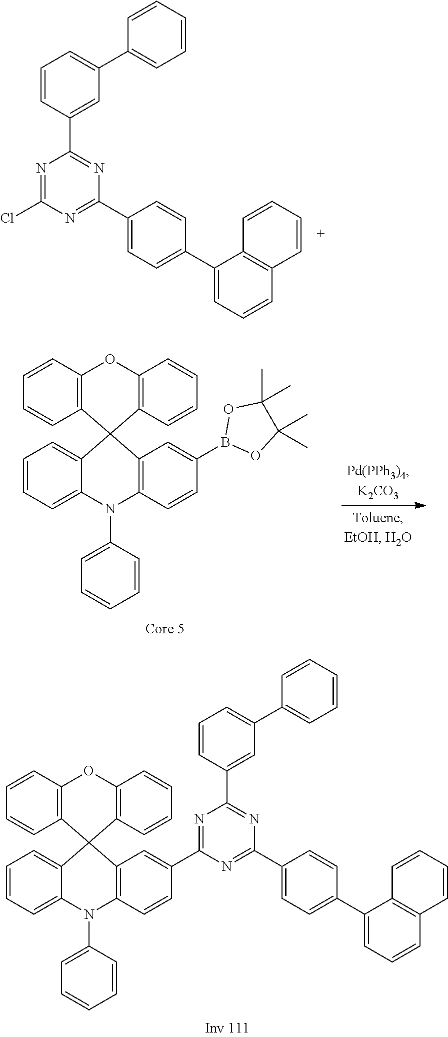 [SMILES: Clc1nc(-c2ccc(-c3cccc4ccccc34)cc2)nc(-c2cccc(-c3ccccc3)c2)n1 ; CC1(C)OB(c2ccc3c(c2)C2(c4ccccc4Oc4ccccc42)c2ccccc2N3c2ccccc2)OC1(C)C ; c1ccc(-c2cccc(-c3nc(-c4ccc(-c5cccc6ccccc56)cc4)nc(-c4ccc5c(c4)C4(c6ccccc6Oc6ccccc64)c4ccccc4N5c4ccccc4)n3)c2)cc1]

Except that 2-([1,1′-biphenyl]-3-yl)-4-chloro-6-(4-(naphthalen-1-yl)phenyl)-1,3,5-triazine (15.0 g 31.9 mmol) was used instead of 2-(6-chloro-2-phenylpyrimidin-4-yl)-9,9-diphenyl-9H-fluorene-3-carbonitrile, Inv 111 (19.8 g, yield 72.4%) was obtained by performing the same procedure as in Synthesis Example 8.

Mass: [(M+H)⁺]: 858

### [Synthesis Example 18] Synthesis of Inv 113

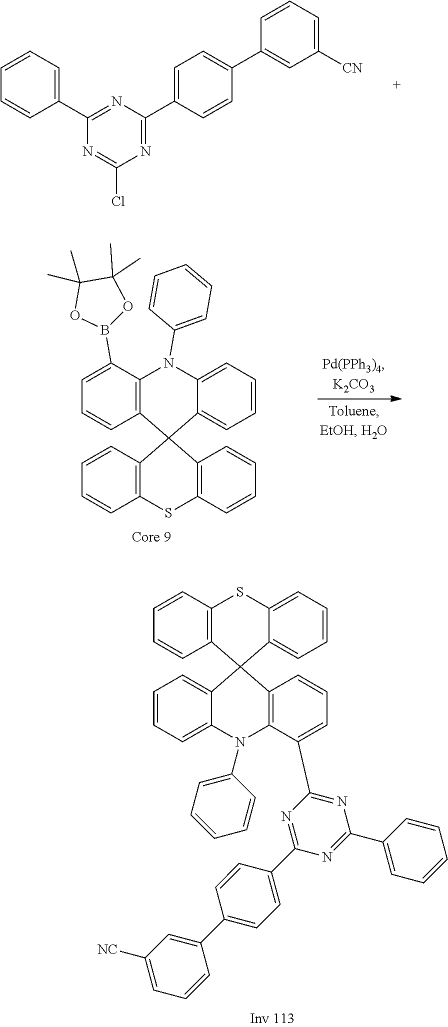

Except that 4′-(4-chloro-6-phenyl-1,3,5-triazin-2-yl)-[1,1′-biphenyl]-3-carbonitrile (15.0 g 40.7 mmol) was used instead of 3-(4-chloro-6-(4-cyanophenyl)-1,3,5-triazin-2-yl)benzonitrile, Inv 113 (22.5 g, yield 71.7%) was obtained by performing the same procedure as in Preparation Example 16.

Mass [(M+H)⁺]: 772

### [Synthesis Example 19] Synthesis of Inv 115

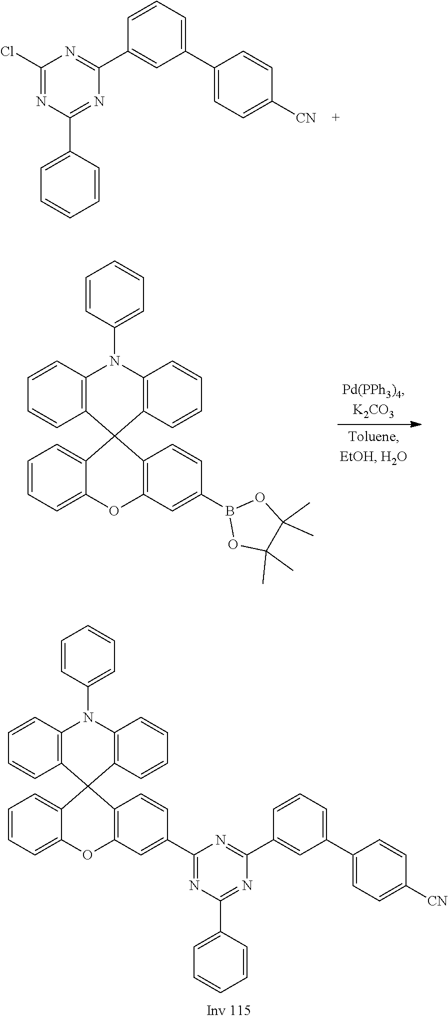 [SMILES: N#Cc1ccc(-c2cccc(-c3nc(Cl)nc(-c4ccccc4)n3)c2)cc1 ; CC1(C)OB(c2ccc3c(c2)Oc2ccccc2C32c3ccccc3N(c3ccccc3)c3ccccc32)OC1(C)C ; N#Cc1ccc(-c2cccc(-c3nc(-c4ccccc4)nc(-c4ccc5c(c4)Oc4ccccc4C54c5ccccc5N(c5ccccc5)c5ccccc54)n3)c2)cc1]

3′-(4-chloro-6-phenyl-1,3,5-triazin-2-yl)-[1,1′-biphenyl]-4-carbonitrile (15.0 g, 40.7 mmol), 10-phenyl-3′-(4,4,5,5-tetramethyl-1,3,2-dioxaborolan-2-yl)-10H-spiro[acridine-9,9′-xanthene](24.6 g, 44.7 mmol), Pd(Pph₃)₄ (1.4 g, 1.2 mmol), and K₂CO₃ (16.9 g, 122.0 mmol) were added to 160 ml of Toluene, 40 ml of EtOH, and 40 ml of H₂O, and the resulting mixture was heated to reflux for 4 hours. After the reaction was completed, the resulting mixture was extracted with dichloromethane, MgSO₄ was added thereto, and filtered. After removing the solvent of the filtered organic layer, the resulting mixture was subjected to column chromatography using dichloromethane and hexane, and then recrystallized with toluene acetone to obtain Inv 115 (24.0 g, yield 78.0%).

Mass: [(M+H)⁺]: 756

### [Examples 1 to 19] Manufacturing of Blue Organic EL Devices

The compounds synthesized in the above synthesis examples were subjected to high-purity sublimation purification in a conventionally known method, and then blue organic EL devices were manufactured according to the following procedure.

A glass substrate thin-film-coated with indium tin oxide (ITO) to a thickness of 1,200 Å was washed with distilled water ultrasonically. After washing with distilled water was completed, the glass substrate was ultrasonically washed with a solvent, such as isopropyl alcohol, acetone and methanol, dried, transferred to a UV OZONE cleaner (Power sonic 405, Hwasin Tech), cleaned for 5 minutes using UV, and then transferred to a vacuum evaporator.

On the ITO transparent electrode prepared as above, HT-1+2% HAT-CN (100 Å)/HT-1 (1400 Å)/HT-2 (50 Å)/BH+2% BD (200 Å)/ET-2 (50 Å)/each compounds Inv 6 to Inv 103: LiQ=1:1 (300 Å)/LiF (10 Å)/Al (1000 Å) were stacked in order to manufacture an organic EL device.

In such a case, the structures of HT-1, HAT-CN, HT-2, BH, BD, ET-1, ET-2 and LiQ are each as follows.

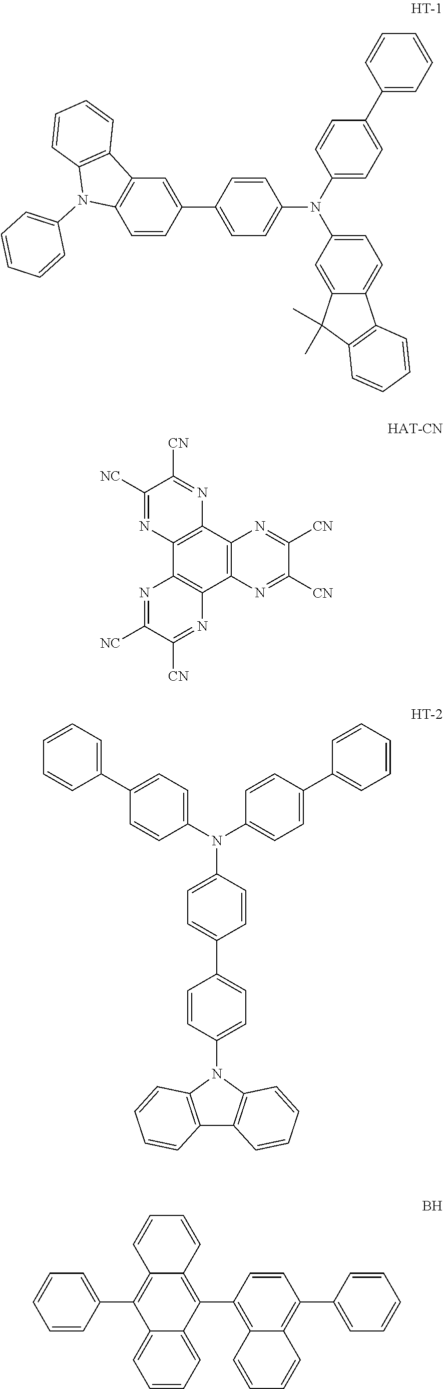

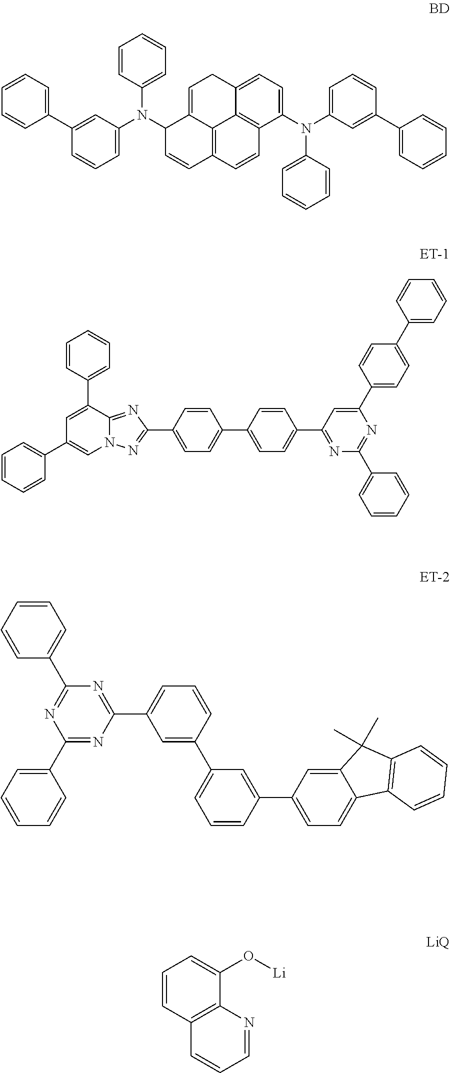

### [Comparative Examples 1 to 4] Manufacturing of Blue Organic EL Device

A blue organic EL device of Comparative Examples 1 to 4 were manufactured in the same manner as in Example 1, except that Compounds ET-1, ET-3, ET-4 and ET-5 was deposited at 300 Å instead of Compound 1 used as the electron transport layer material in Example 1.

In such a case, the structures of ET-3, ET-4 and ET-5 are each as follows.

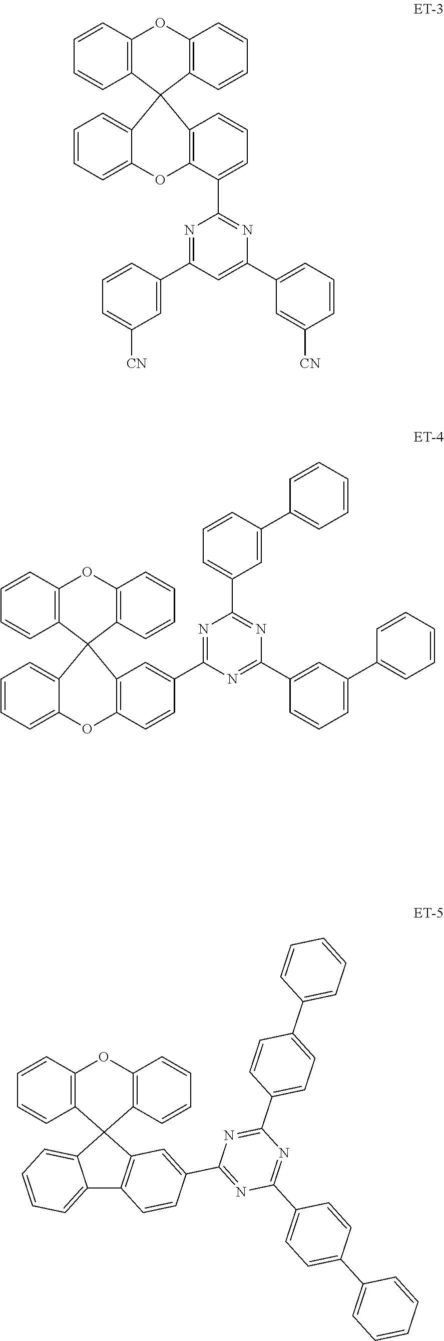 [SMILES: N#Cc1cccc(-c2cc(-c3cccc(C#N)c3)nc(-c3cccc4c3Oc3ccccc3C43c4ccccc4Oc4ccccc43)n2)c1 ; c1ccc(-c2cccc(-c3nc(-c4cccc(-c5ccccc5)c4)nc(-c4ccc5c(c4)C4(c6ccccc6Oc6ccccc64)c4ccccc4O5)n3)c2)cc1 ; c1ccc(-c2ccc(-c3nc(-c4ccc(-c5ccccc5)cc4)nc(-c4ccc5c(c4)C4(c6ccccc6Oc6ccccc64)c4ccccc4-5)n3)cc2)cc1]

### Evaluation Example 1

For each of the blue organic EL devices manufactured in Examples (Ex.) 1 to 19 and Comparative Examples (Comp. Ex.) 1 to 4, a driving voltage, a current efficiency at a current density of 10 mA/cm², T₉₅ and an emission peak were measured, and the results are shown in Table 1 below.

TABLE 1

| | | Driving | | | |
|---|---|---|---|---|---|
| | Electron | voltage | EL | Current | |
| | transport | [symbol] | peak | efficiency | T₉₅ |
| Sample | layer | (V) | (nm) | (cd/A) | (hr) |
| Ex. 1 | Inv 6 | 4.2 | 460 | 6.4 | 115 |
| Ex. 2 | Inv 7 | 4.2 | 461 | 6.6 | 125 |
| Ex. 3 | Inv 8 | 4.4 | 460 | 6.4 | 140 |
| Ex. 4 | Inv 13 | 4.1 | 460 | 7.0 | 90 |
| Ex. 5 | Inv 29 | 4.7 | 461 | 6.4 | 135 |
| Ex. 6 | Inv 40 | 4.2 | 459 | 6.7 | 95 |
| Ex. 7 | Inv 41 | 4.4 | 459 | 6.6 | 90 |
| Ex. 8 | Inv 44 | 4.1 | 460 | 6.8 | 95 |
| Ex. 9 | Inv 46 | 4.4 | 461 | 6.4 | 130 |
| Ex. 10 | Inv 54 | 4.5 | 458 | 6.5 | 138 |
| Ex. 11 | Inv 56 | 4.2 | 460 | 6.5 | 120 |
| Ex. 12 | Inv 63 | 4.2 | 462 | 6.9 | 100 |
| Ex. 13 | Inv 78 | 4.5 | 462 | 6.4 | 135 |
| Ex. 14 | Inv 86 | 4.4 | 458 | 6.3 | 110 |
| Ex. 15 | Inv 97 | 4.3 | 461 | 6.5 | 130 |
| Ex. 16 | Inv 100 | 4.2 | 460 | 6.7 | 110 |
| Ex. 17 | Inv 111 | 4.3 | 459 | 6.9 | 115 |
| Ex. 18 | Inv 113 | 4.4 | 458 | 6.6 | 125 |
| Ex. 19 | Inv 115 | 4.7 | 462 | 6.3 | 150 |
| Comp. Ex. 1 | ET-1 | 4.7 | 459 | 5.9 | 80 |
| Comp. Ex. 2 | ET-3 | 4.6 | 461 | 5.8 | 45 |
| Comp. Ex. 3 | ET-4 | 5.2 | 460 | 6.1 | 55 |
| Comp. Ex. 4 | ET-5 | 5.1 | 459 | 5.0 | 90 |

As shown in Table 1, it was appreciated that the blue organic EL devices of Examples 1 to 19 using the compound according to the present invention as an electron transport layer material exhibit significantly excellent performance in terms of driving voltage, emission peak and current efficiency compared to the blue organic EL device of Comparative Examples 1 to 4 using conventional compound ET-1, ET-3, ET-4 and ET-5 as an electron transport layer material.

1-15. (canceled)

16. A compound represented by Chemical Formula 1: 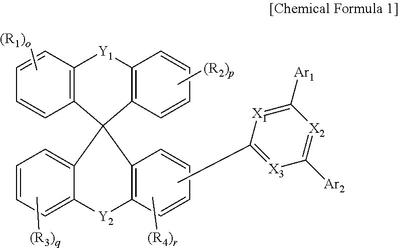
wherein in Chemical Formula 1,
X₁ to X₃ are the same as or different from each other and are each independently C(R₅) or N, provided that at least two of X₁ to X₃ are N;
Y₁ and Y₂ are the same as or different from each other and are each independently selected from the group consisting of O, S, and NR₁₁, provided that the case where both Y₁ and Y₂ are O is excluded;
Ar₁ and Ar₂ are the same as or different from each other and are each independently selected from the group consisting of a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 ring atoms, a C₆-C₆₀ aryl group, a heteroaryl group having 5 to 60 ring atoms, a C₁-C₄₀ alkyloxy group, a C₆-C₆₀ aryloxy group, a C₃-C₄₀ alkylsilyl group, a C₆-C₆₀ arylsilyl group, a C₁-C₄₀ alkylboron group, a C₆-C₆₀ arylboron group, a C₆-C₆₀ arylphosphine group, a C₆-C₆₀ arylphosphine oxide group, and a C₆-C₆₀ arylamine group, or combine with an adjacent group to form a fused ring;
R₁ to R₅ and R₁₁ are the same as or different from one another and are each independently selected from the group consisting of a hydrogen, a deuterium (D), a halogen, a cyano group, a nitro group, a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 ring atoms, a C₆-C₆₀ aryl group, a heteroaryl group having 5 to 60 ring atoms, a C₁-C₄₀ alkyloxy group, a C₆-C₆₀ aryloxy group, a C₃-C₄₀ alkylsilyl group, a C₆-C₆₀ arylsilyl group, a C₁-C₄₀ alkylboron group, a C₆-C₆₀ arylboron group, a C₆-C₆₀ aryl phosphine group, a C₆-C₆₀ arylphosphine oxide group, and a C₆-C₆₀ arylamine group, or combine with an adjacent group to form a fused ring;
o, p, and q are each independently an integer of 0 to 4, and r is an integer of 0 to 3; and
the alkyl group, alkenyl group, alkynyl group, aryl group, heteroaryl group, aryloxy group, alkyloxy group, cycloalkyl group, heterocycloalkyl group, alkylsilyl group, arylsilyl group, alkylboron group, arylboron group, arylphosphine group, arylphosphine oxide group, and arylamine group of Ar₁ to Ar₂, R₁ to R₅, and R₁ may be each independently substituted with at least one substituent selected from the group consisting of a deuterium (D), a halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 ring atoms, a C₆ to C₆₀ aryl group, a heteroaryl group having 5 to 60 ring atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, and wherein when the substituent is present in a plural number, they may be the same or different from each other.

17. The compound of claim 16, wherein the Y₁ and Y₂ containing ring is selected from the group of substituents represented by the following structural formulas: 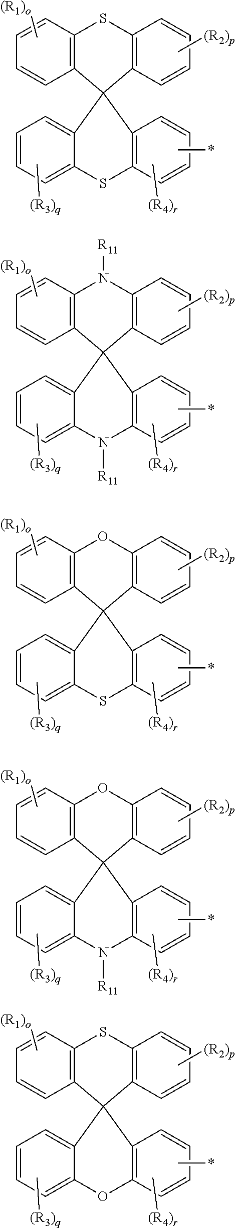 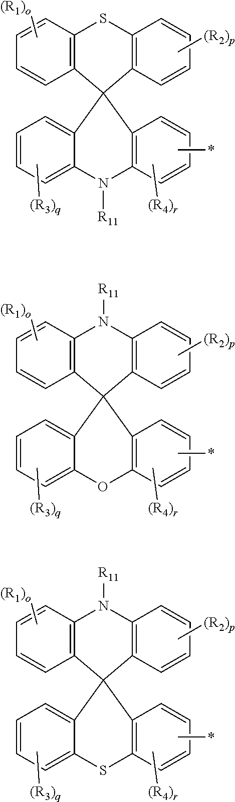
wherein in the formulas,
* indicates a site where a bond with Chemical Formula 1 is made,
R₁₁ is selected from the group consisting of a hydrogen, a deuterium (D), a C₁ to C₄₀ alkyl group, a C₆ to C₃₀ aryl group, and a heteroaryl group having 5 to 30 nuclear atoms,
R₁ to R₄, o, p, q and r are the same as defined in claim 16.

18. The compound of claim 16, wherein Y₁ and Y₂ are different from each other.

19. The compound of claim 16, wherein the X-containing ring is selected from the group of substituents represented by the following structural formulas: 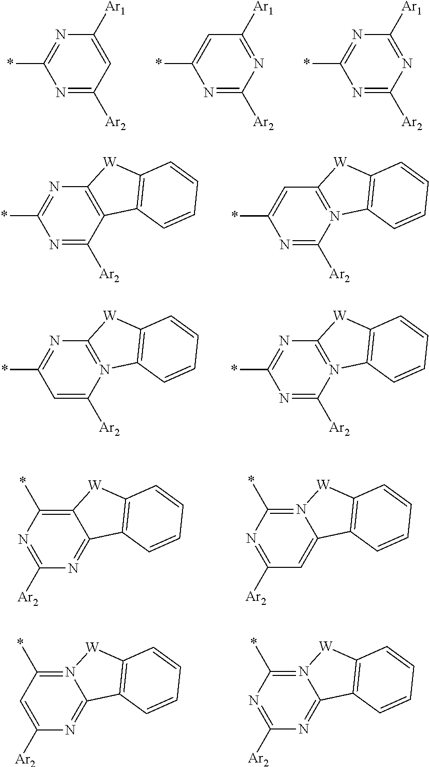
wherein in the formulas,
* indicates a site where a bond with Chemical Formula 1 is made,
W is 0 or S, and
Ar₁ and Ar₂ are the same as defined in claim 16.

20. The compound of claim 16, wherein Ar₁ and Ar₂ are the same as or different from each other, and are each independently selected from the group consisting of a C₆ to C₆₀ aryl group and a heteroaryl group having 5 to 60 nuclear atoms.

21. The compound of claim 16, wherein Ar₁ and Ar₂ are the same as or different from each other, and are each independently selected from the following structural formulas: 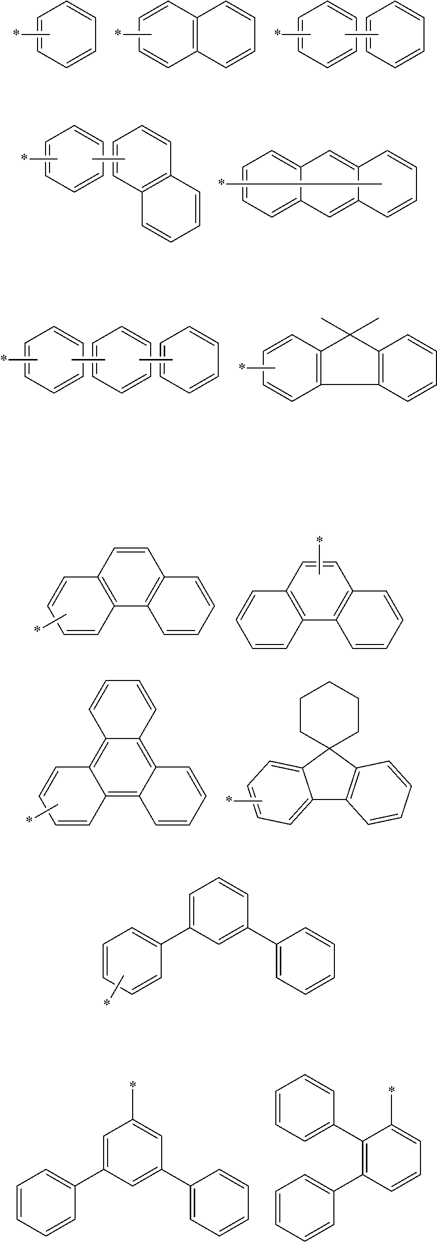 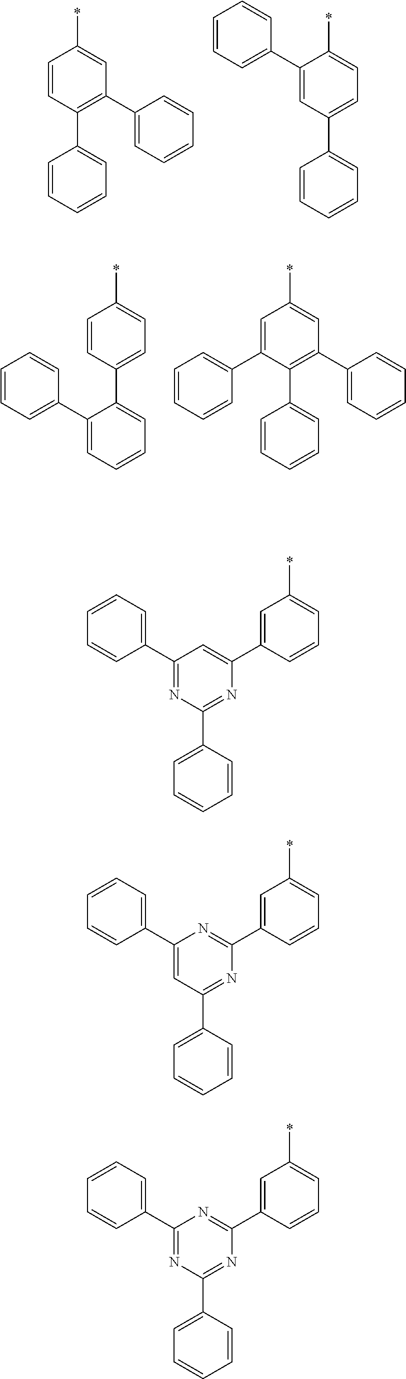 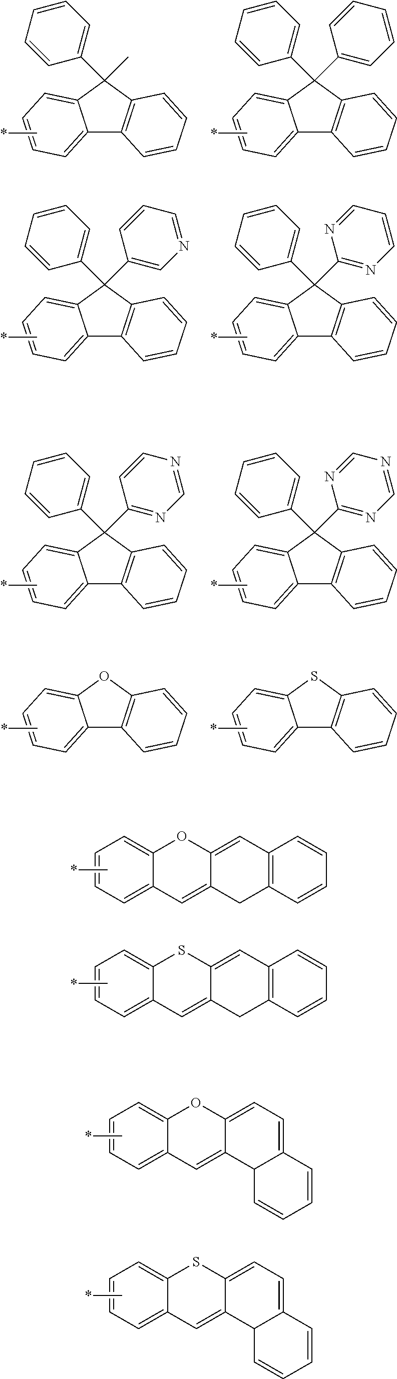 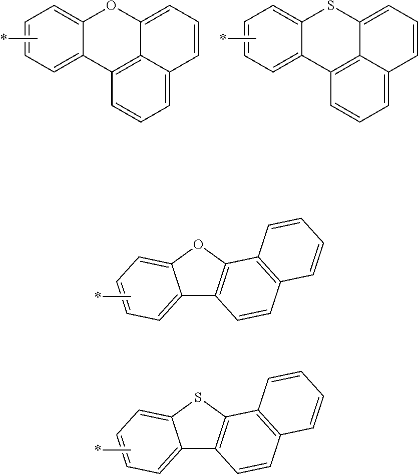
wherein in the formulas,
* indicates a site where a bond with Chemical Formula 1 is made.

22. The compound of claim 16, wherein R₁ to R₄ are the same or different from each other and are each independently selected from the group consisting of a hydrogen, a deuterium (D), a cyano group, a C₁ to C₄₀ alkyl group, a C₆ to C₆₀ aryl group, and a heteroaryl group having 5 to 60 nuclear atoms, or combine with an adjacent group to form a fused ring.

23. The compound of claim 16, wherein the compound represented by Chemical Formula 1 is a compound represented by any one of Chemical Formula 2 to Chemical Formula 9 below: 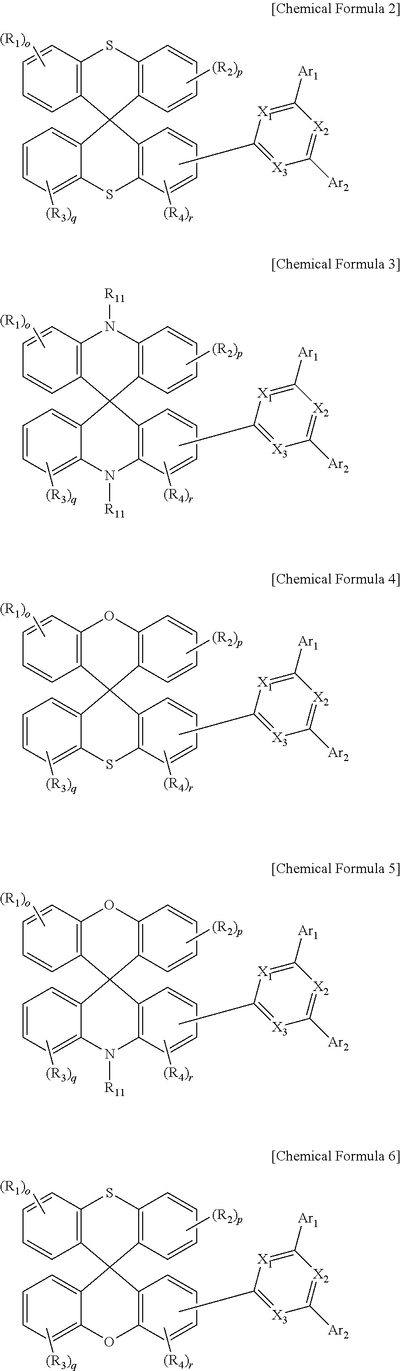 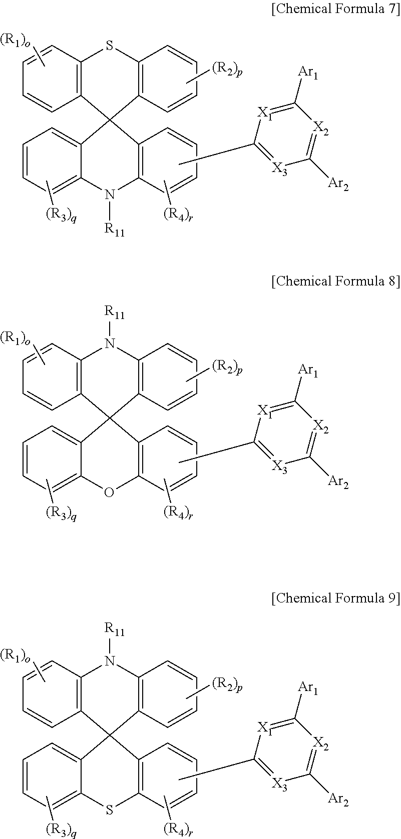
wherein the formulas,
X₁ to X₃, Ar₁ to Ar₂, R₁ to R₄, R₁₁, o, p, q and r are the same as defined in claim 16.

24. The compound of claim 16, wherein the compound represented by Chemical Formula 1 is a compound represented by any one of Chemical Formula 10 to Chemical Formula 20 below: 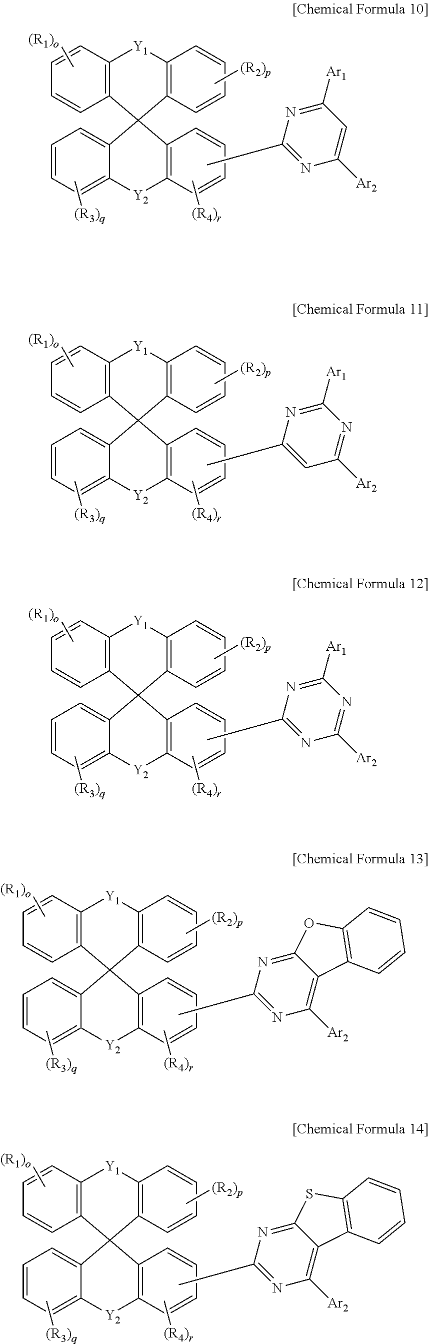 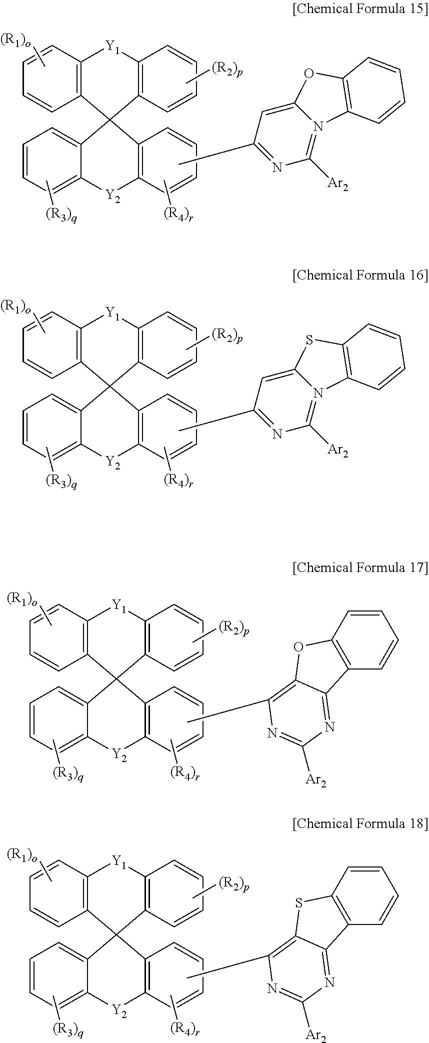 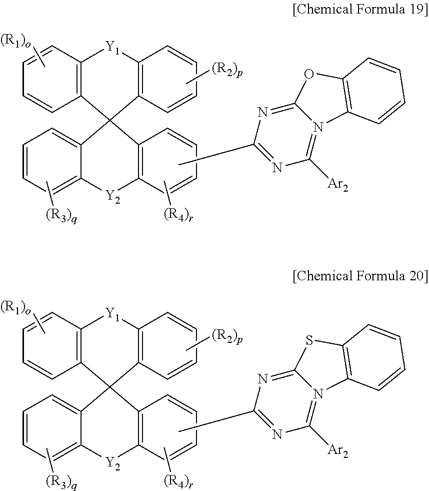
wherein the formulas,
Y₁ to Y₂, Ar₁ to Ar₂, R₁ to R₄, o, p, q and r are the same as defined in claim 16.

25. The compound of claim 16, wherein the compound represented by Chemical Formula 1 is a compound represented by any one of Chemical Formula 21 to Chemical Formula 28 below: 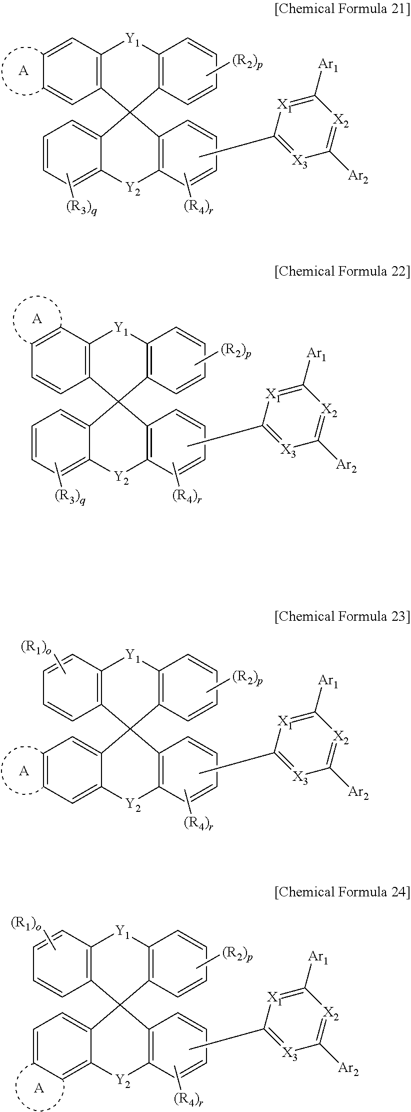 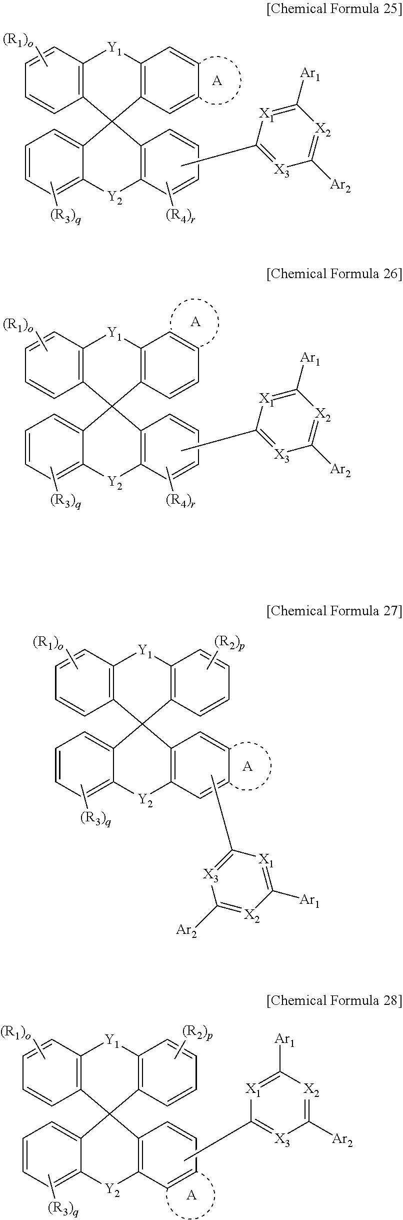
wherein the formulas,
Ring A is a monocyclic or polycyclic alicyclic ring, a monocyclic or polycyclic heteroalicyclic ring, a monocyclic or polycyclic aromatic ring, or a monocyclic or polycyclic heteroaromatic ring,
X₁ to X₃, Y₁ to Y₂, Ar₁ to Ar₂, R₁ to R₄, o, p, q and r are the same as defined in claim 16.

26. The compound of claim 16, wherein the compound represented by Chemical Formula 1 is a compound represented by any one of Chemical Formula 29 to Chemical Formula 31 below: 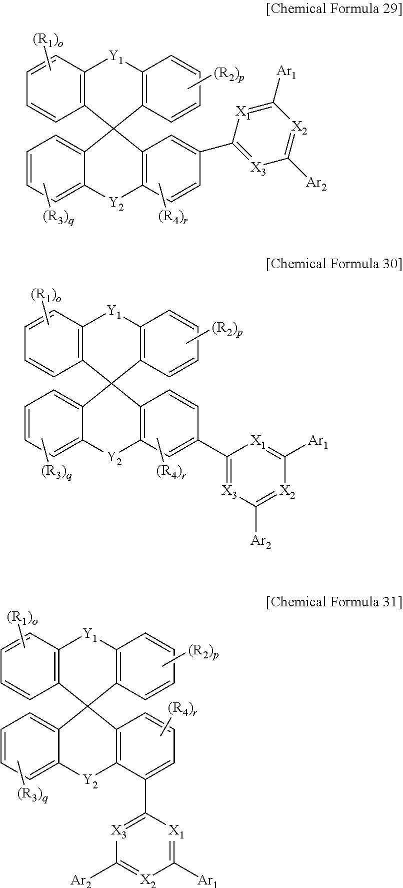
wherein the formulas,
X₁ to X₃, Y₁ to Y₂, Ar₁ to Ar₂, R₁ to R₄, o, p, q and r are the same as defined in claim 16.

27. The compound of claim 16, wherein the compound represented by Chemical Formula 1 is selected from compounds represented by any one of the following Chemical Formulas 1 to 120:

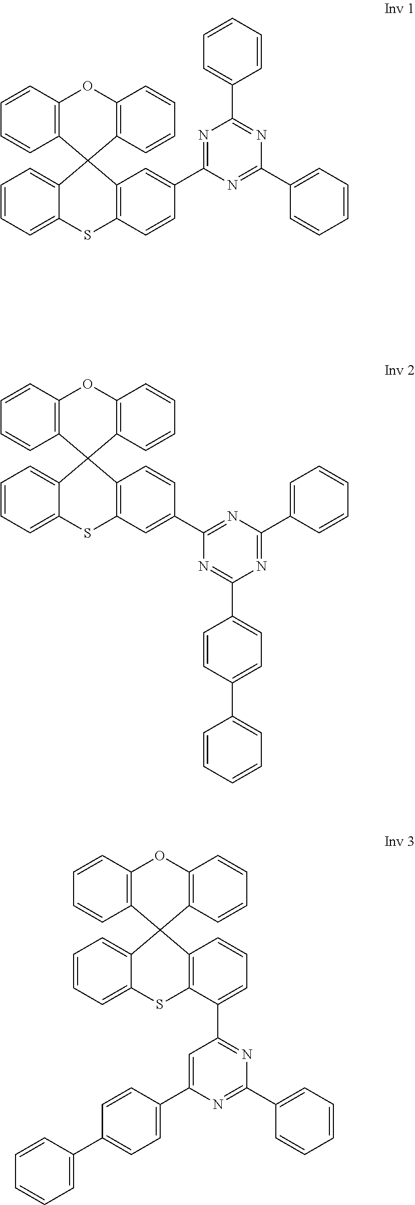 [SMILES: c1ccc(-c2nc(-c3ccccc3)nc(-c3ccc4c(c3)C3(c5ccccc5Oc5ccccc53)c3ccccc3S4)n2)cc1 ; c1ccc(-c2ccc(-c3nc(-c4ccccc4)nc(-c4ccc5c(c4)Sc4ccccc4C54c5ccccc5Oc5ccccc54)n3)cc2)cc1 ; c1ccc(-c2ccc(-c3cc(-c4cccc5c4Sc4ccccc4C54c5ccccc5Oc5ccccc54)nc(-c4ccccc4)n3)cc2)cc1]

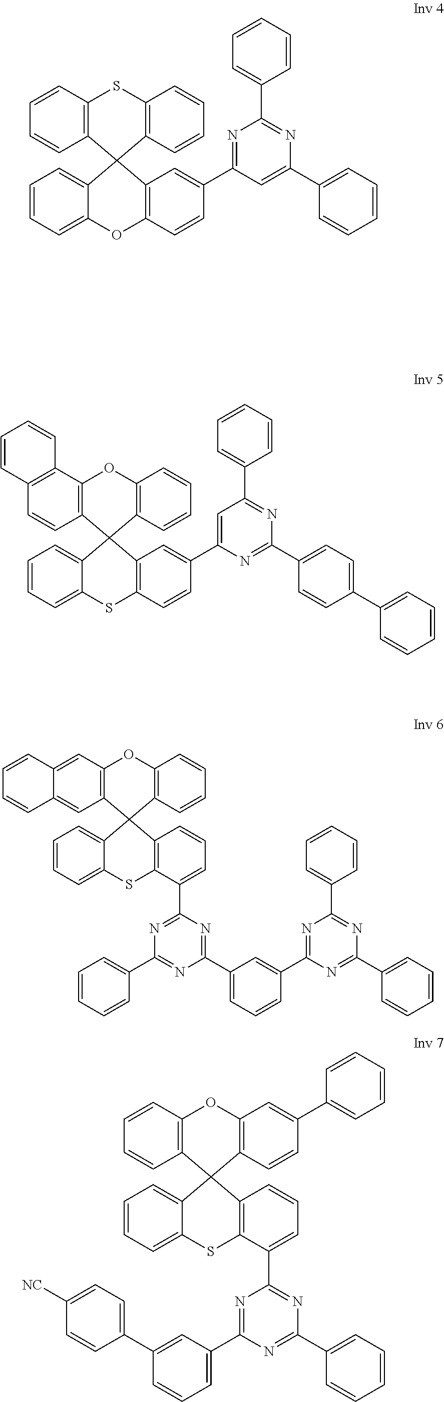 [SMILES: c1ccc(-c2cc(-c3ccc4c(c3)C3(c5ccccc5O4)c4ccccc4Sc4ccccc43)nc(-c3ccccc3)n2)cc1 ; c1ccc(-c2ccc(-c3nc(-c4ccccc4)cc(-c4ccc5c(c4)C4(c6ccccc6Oc6c4ccc4ccccc64)c4ccccc4S5)n3)cc2)cc1 ; c1ccc(-c2nc(-c3ccccc3)nc(-c3cccc(-c4nc(-c5ccccc5)nc(-c5cccc6c5Sc5ccccc5C65c6ccccc6Oc6cc7ccccc7cc65)n4)c3)n2)cc1 ; N#Cc1ccc(-c2cccc(-c3nc(-c4ccccc4)nc(-c4cccc5c4Sc4ccccc4C54c5ccccc5Oc5cc(-c6ccccc6)ccc54)n3)c2)cc1]

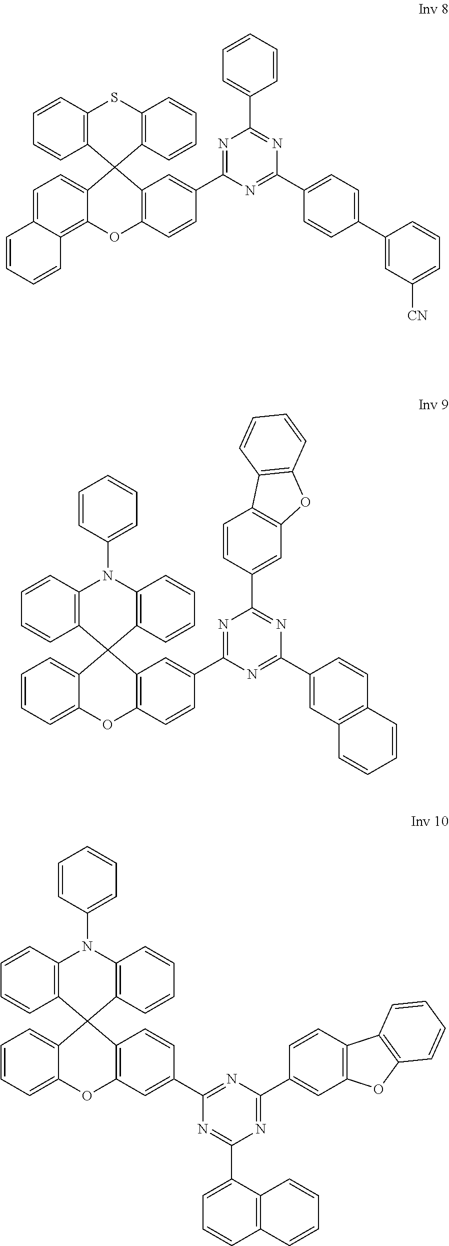 [SMILES: N#Cc1cccc(-c2ccc(-c3nc(-c4ccccc4)nc(-c4ccc5c(c4)C4(c6ccccc6Sc6ccccc64)c4ccc6ccccc6c4O5)n3)cc2)c1 ; c1ccc(N2c3ccccc3C3(c4ccccc4Oc4ccc(-c5nc(-c6ccc7ccccc7c6)nc(-c6ccc7c(c6)oc6ccccc67)n5)cc43)c3ccccc32)cc1 ; c1ccc(N2c3ccccc3C3(c4ccccc4Oc4cc(-c5nc(-c6ccc7c(c6)oc6ccccc67)nc(-c6cccc7ccccc67)n5)ccc43)c3ccccc32)cc1]

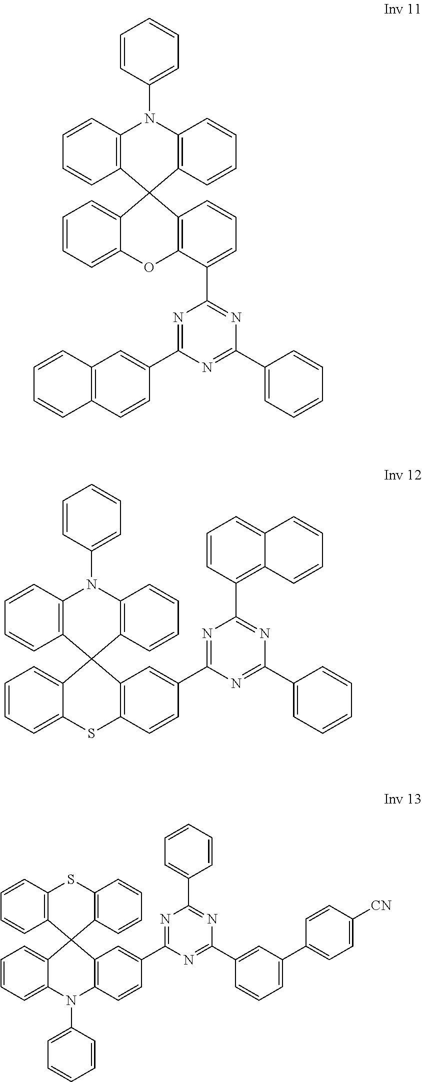 [SMILES: c1ccc(-c2nc(-c3ccc4ccccc4c3)nc(-c3cccc4c3Oc3ccccc3C43c4ccccc4N(c4ccccc4)c4ccccc43)n2)cc1 ; c1ccc(-c2nc(-c3ccc4c(c3)C3(c5ccccc5S4)c4ccccc4N(c4ccccc4)c4ccccc43)nc(-c3cccc4ccccc34)n2)cc1 ; N#Cc1ccc(-c2cccc(-c3nc(-c4ccccc4)nc(-c4ccc5c(c4)C4(c6ccccc6Sc6ccccc64)c4ccccc4N5c4ccccc4)n3)c2)cc1]

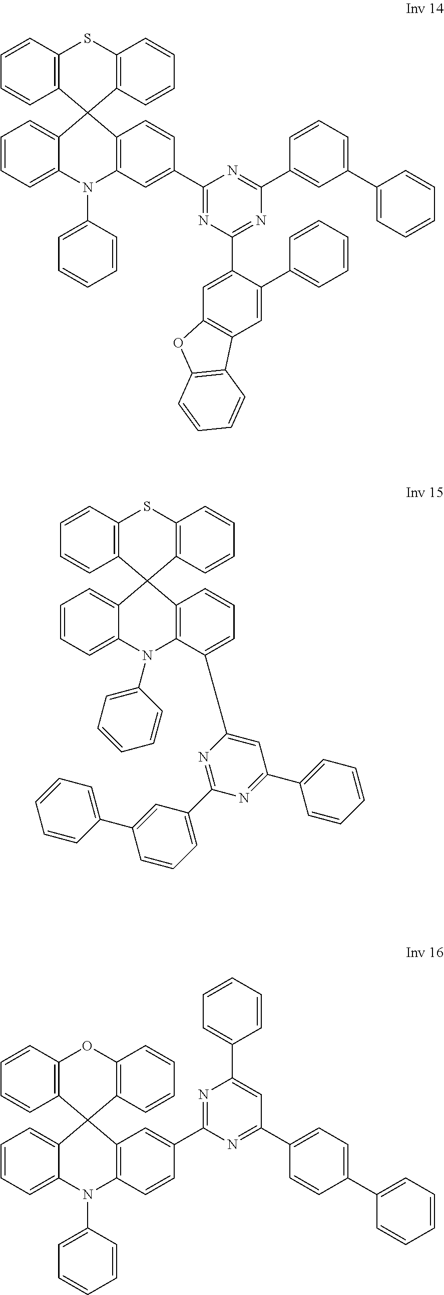 [SMILES: c1ccc(-c2cccc(-c3nc(-c4ccc5c(c4)N(c4ccccc4)c4ccccc4C54c5ccccc5Sc5ccccc54)nc(-c4cc5oc6ccccc6c5cc4-c4ccccc4)n3)c2)cc1 ; c1ccc(-c2cccc(-c3nc(-c4ccccc4)cc(-c4cccc5c4N(c4ccccc4)c4ccccc4C54c5ccccc5Sc5ccccc54)n3)c2)cc1 ; c1ccc(-c2ccc(-c3cc(-c4ccccc4)nc(-c4ccc5c(c4)C4(c6ccccc6Oc6ccccc64)c4ccccc4N5c4ccccc4)n3)cc2)cc1]

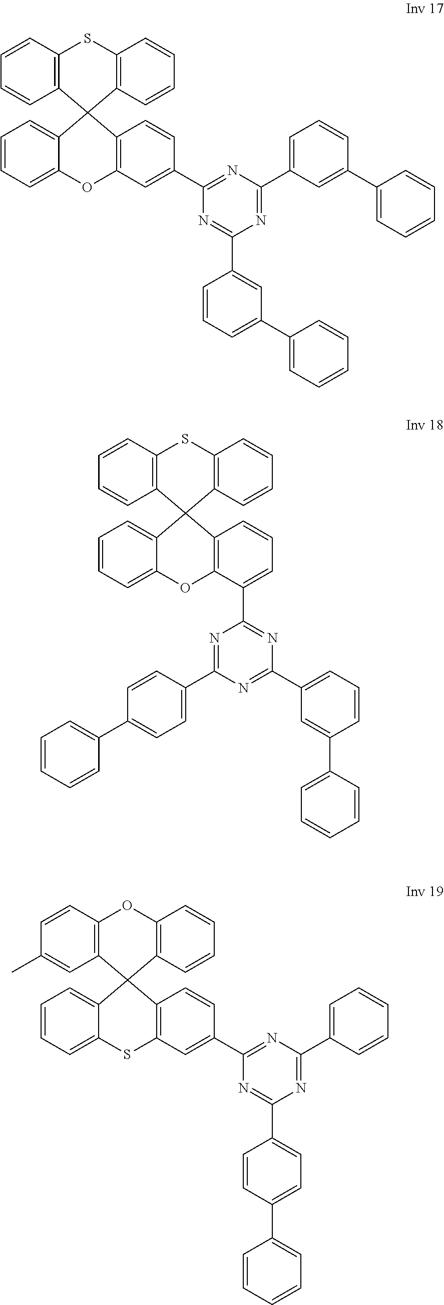 [SMILES: c1ccc(-c2cccc(-c3nc(-c4cccc(-c5ccccc5)c4)nc(-c4ccc5c(c4)Oc4ccccc4C54c5ccccc5Sc5ccccc54)n3)c2)cc1 ; c1ccc(-c2ccc(-c3nc(-c4cccc(-c5ccccc5)c4)nc(-c4cccc5c4Oc4ccccc4C54c5ccccc5Sc5ccccc54)n3)cc2)cc1 ; Cc1ccc2c(c1)C1(c3ccccc3O2)c2ccccc2Sc2cc(-c3nc(-c4ccccc4)nc(-c4ccc(-c5ccccc5)cc4)n3)ccc21]

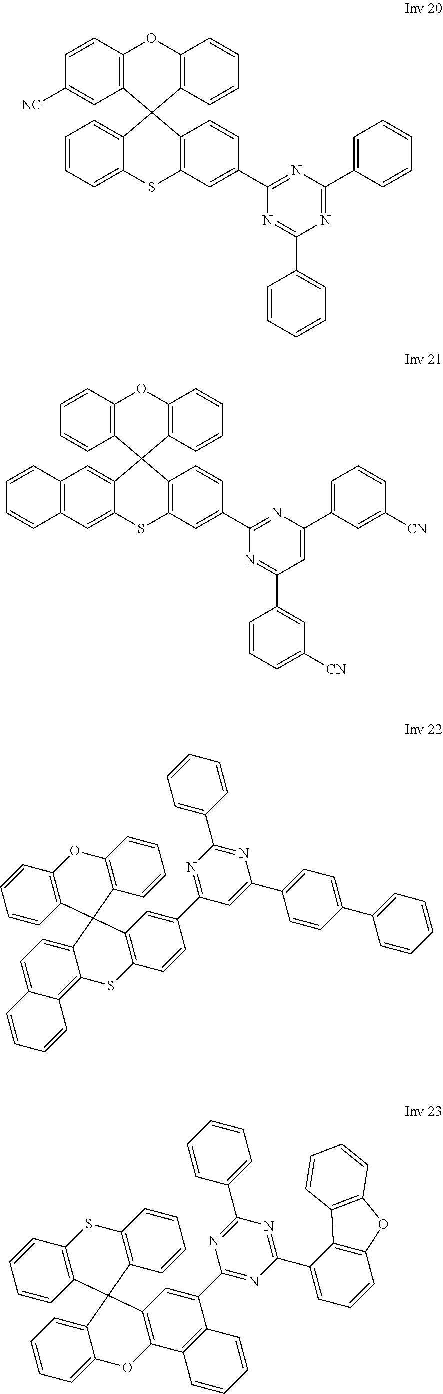 [SMILES: N#Cc1ccc2c(c1)C1(c3ccccc3O2)c2ccccc2Sc2cc(-c3nc(-c4ccccc4)nc(-c4ccccc4)n3)ccc21 ; N#Cc1cccc(-c2cc(-c3cccc(C#N)c3)nc(-c3ccc4c(c3)Sc3cc5ccccc5cc3C43c4ccccc4Oc4ccccc43)n2)c1 ; c1ccc(-c2ccc(-c3cc(-c4ccc5c(c4)C4(c6ccccc6Oc6ccccc64)c4ccc6ccccc6c4S5)nc(-c4ccccc4)n3)cc2)cc1 ; c1ccc(-c2nc(-c3cc4c(c5ccccc35)Oc3ccccc3C43c4ccccc4Sc4ccccc43)nc(-c3cccc4oc5ccccc5c34)n2)cc1]

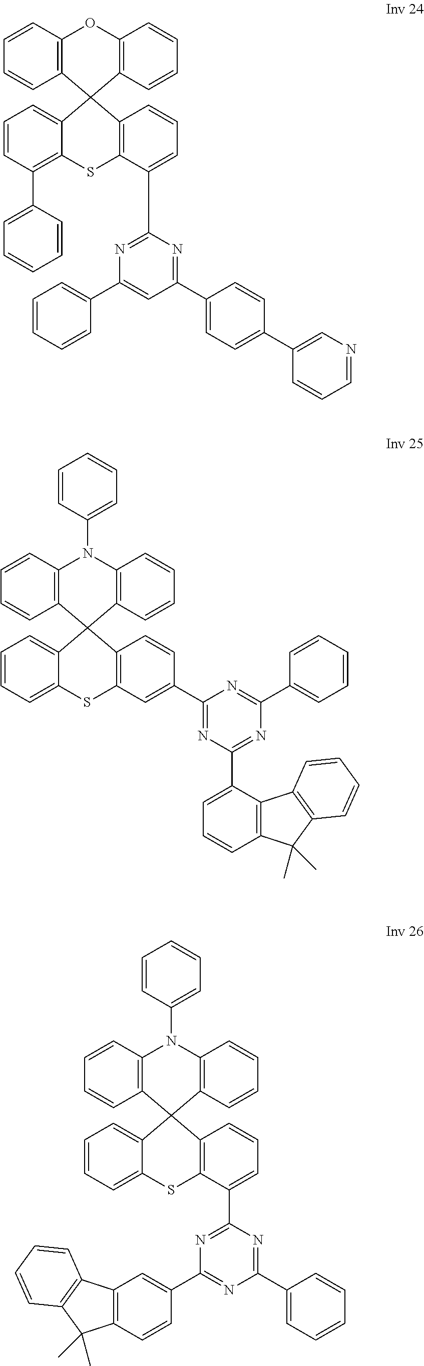 [SMILES: c1ccc(-c2cc(-c3ccc(-c4cccnc4)cc3)nc(-c3cccc4c3Sc3c(-c5ccccc5)cccc3C43c4ccccc4Oc4ccccc43)n2)cc1 ; CC1(C)c2ccccc2-c2c(-c3nc(-c4ccccc4)nc(-c4ccc5c(c4)Sc4ccccc4C54c5ccccc5N(c5ccccc5)c5ccccc54)n3)cccc21 ; CC1(C)c2ccccc2-c2cc(-c3nc(-c4ccccc4)nc(-c4cccc5c4Sc4ccccc4C54c5ccccc5N(c5ccccc5)c5ccccc54)n3)ccc21]

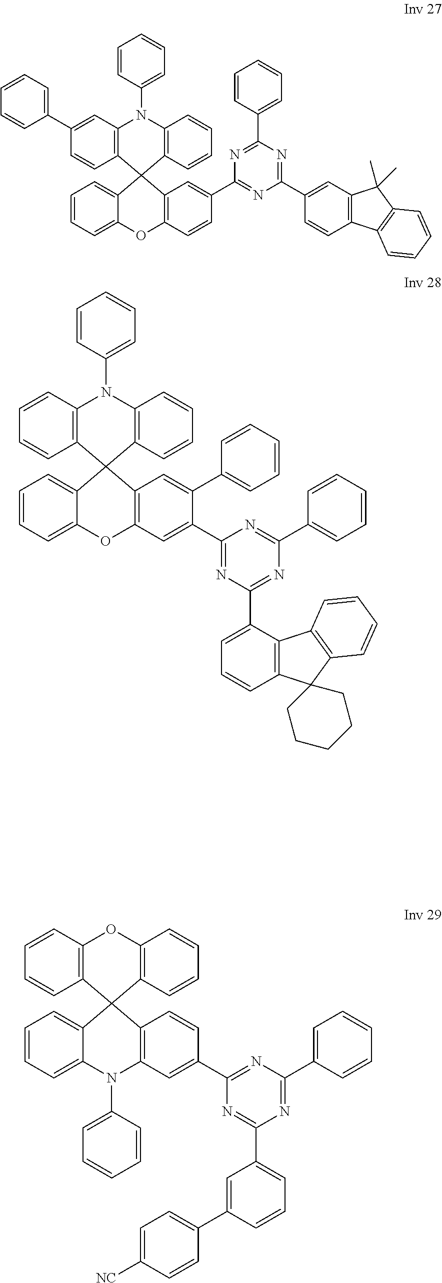 [SMILES: CC1(C)c2ccccc2-c2ccc(-c3nc(-c4ccccc4)nc(-c4ccc5c(c4)C4(c6ccccc6O5)c5ccccc5N(c5ccccc5)c5cc(-c6ccccc6)ccc54)n3)cc21 ; c1ccc(-c2nc(-c3cc4c(cc3-c3ccccc3)C3(c5ccccc5O4)c4ccccc4N(c4ccccc4)c4ccccc43)nc(-c3cccc4c3-c3ccccc3C43CCCCC3)n2)cc1 ; N#Cc1ccc(-c2cccc(-c3nc(-c4ccccc4)nc(-c4ccc5c(c4)N(c4ccccc4)c4ccccc4C54c5ccccc5Oc5ccccc54)n3)c2)cc1]

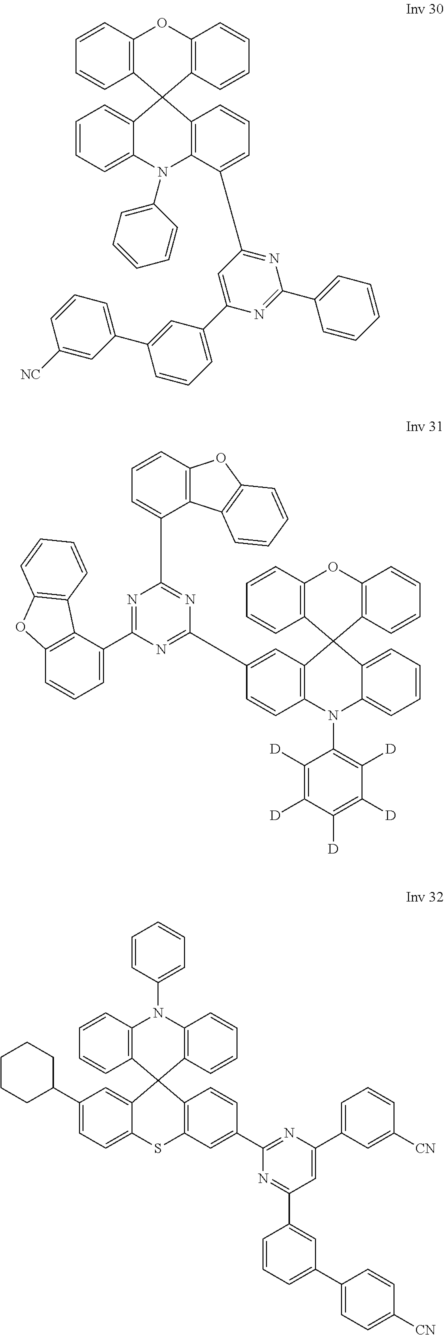 [SMILES: N#Cc1cccc(-c2cccc(-c3cc(-c4cccc5c4N(c4ccccc4)c4ccccc4C54c5ccccc5Oc5ccccc54)nc(-c4ccccc4)n3)c2)c1 ; [2H]c1c([2H])c([2H])c(N2c3ccccc3C3(c4ccccc4Oc4ccccc43)c3cc(-c4nc(-c5cccc6oc7ccccc7c56)nc(-c5cccc6oc7ccccc7c56)n4)ccc32)c([2H])c1[2H] ; N#Cc1ccc(-c2cccc(-c3cc(-c4cccc(C#N)c4)nc(-c4ccc5c(c4)Sc4ccc(C6CCCCC6)cc4C54c5ccccc5N(c5ccccc5)c5ccccc54)n3)c2)cc1]

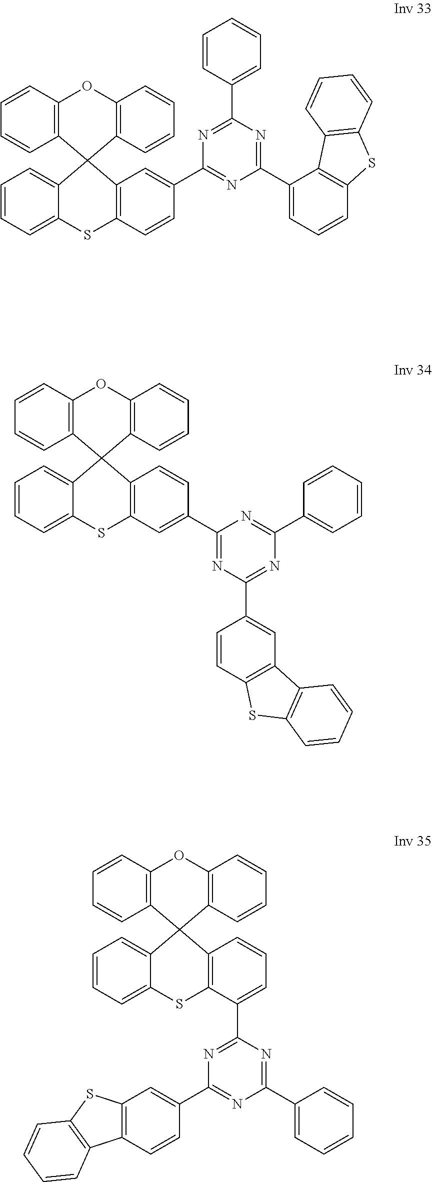 [SMILES: c1ccc(-c2nc(-c3ccc4c(c3)C3(c5ccccc5Oc5ccccc53)c3ccccc3S4)nc(-c3cccc4sc5ccccc5c34)n2)cc1 ; c1ccc(-c2nc(-c3ccc4c(c3)Sc3ccccc3C43c4ccccc4Oc4ccccc43)nc(-c3ccc4sc5ccccc5c4c3)n2)cc1 ; c1ccc(-c2nc(-c3ccc4c(c3)sc3ccccc34)nc(-c3cccc4c3Sc3ccccc3C43c4ccccc4Oc4ccccc43)n2)cc1]

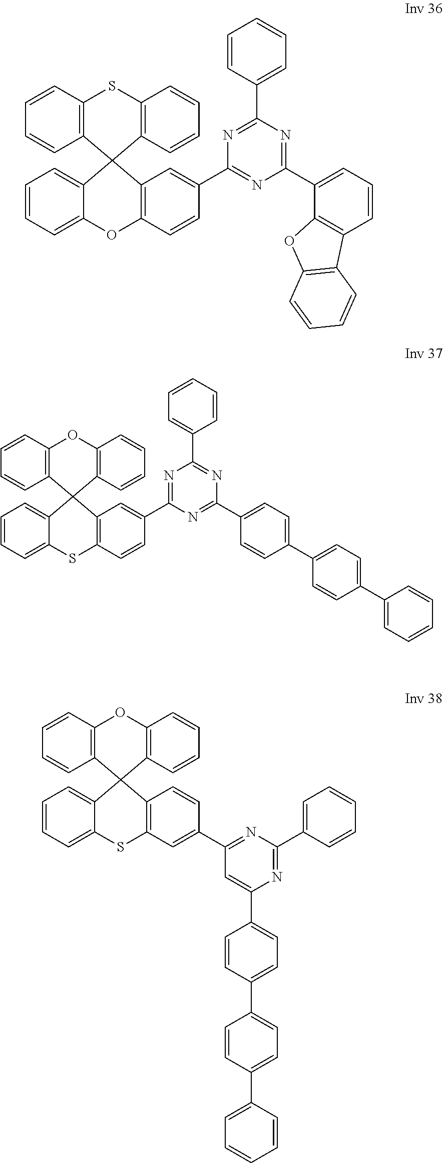 [SMILES: c1ccc(-c2nc(-c3ccc4c(c3)C3(c5ccccc5O4)c4ccccc4Sc4ccccc43)nc(-c3cccc4c3oc3ccccc34)n2)cc1 ; c1ccc(-c2ccc(-c3ccc(-c4nc(-c5ccccc5)nc(-c5ccc6c(c5)C5(c7ccccc7Oc7ccccc75)c5ccccc5S6)n4)cc3)cc2)cc1 ; c1ccc(-c2ccc(-c3ccc(-c4cc(-c5ccc6c(c5)Sc5ccccc5C65c6ccccc6Oc6ccccc65)nc(-c5ccccc5)n4)cc3)cc2)cc1]

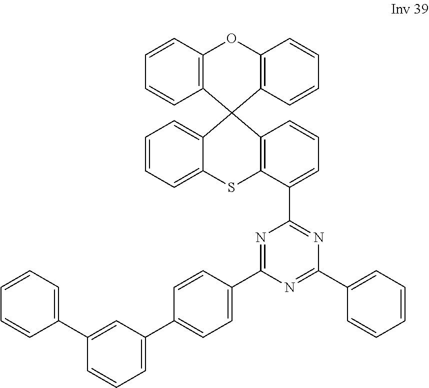 [SMILES: c1ccc(-c2cccc(-c3ccc(-c4nc(-c5ccccc5)nc(-c5cccc6c5Sc5ccccc5C65c6ccccc6Oc6ccccc65)n4)cc3)c2)cc1]

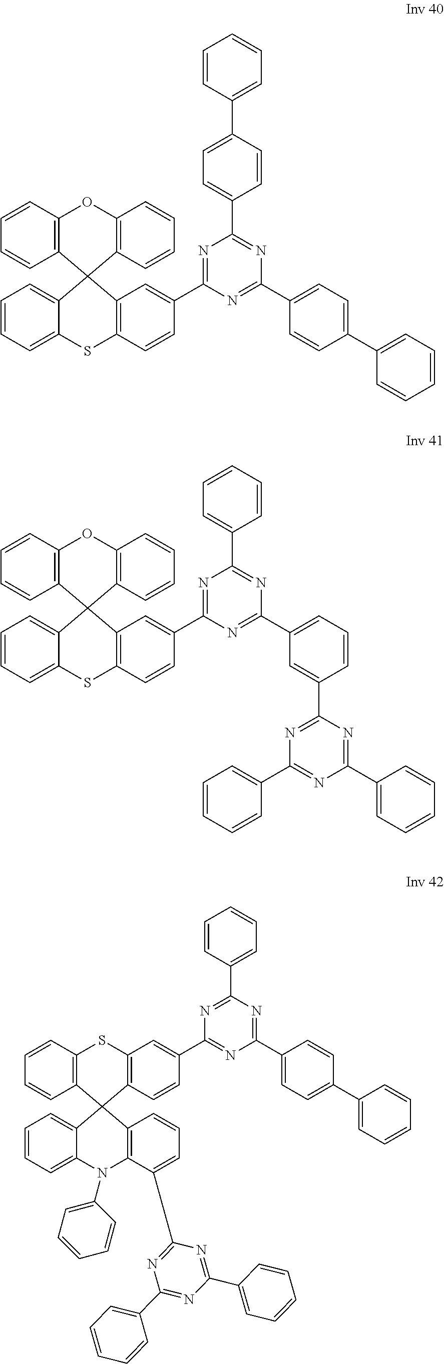 [SMILES: c1ccc(-c2ccc(-c3nc(-c4ccc(-c5ccccc5)cc4)nc(-c4ccc5c(c4)C4(c6ccccc6Oc6ccccc64)c4ccccc4S5)n3)cc2)cc1 ; c1ccc(-c2nc(-c3ccccc3)nc(-c3cccc(-c4nc(-c5ccccc5)nc(-c5ccc6c(c5)C5(c7ccccc7Oc7ccccc75)c5ccccc5S6)n4)c3)n2)cc1 ; c1ccc(-c2ccc(-c3nc(-c4ccccc4)nc(-c4ccc5c(c4)Sc4ccccc4C54c5ccccc5N(c5ccccc5)c5c(-c6nc(-c7ccccc7)nc(-c7ccccc7)n6)cccc54)n3)cc2)cc1]

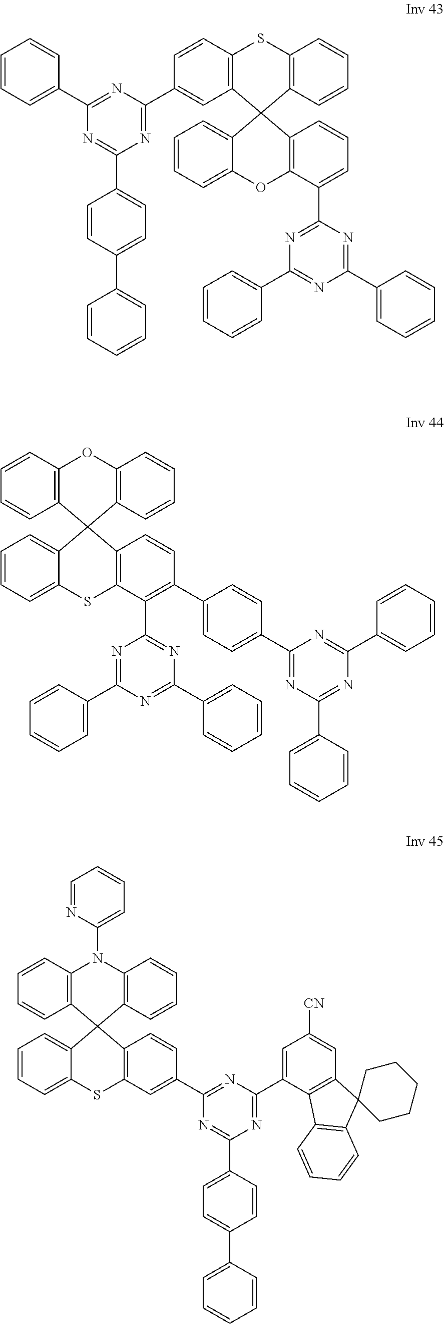 [SMILES: c1ccc(-c2ccc(-c3nc(-c4ccccc4)nc(-c4ccc5c(c4)C4(c6ccccc6Oc6c(-c7nc(-c8ccccc8)nc(-c8ccccc8)n7)cccc64)c4ccccc4S5)n3)cc2)cc1 ; c1ccc(-c2nc(-c3ccccc3)nc(-c3ccc(-c4ccc5c(c4-c4nc(-c6ccccc6)nc(-c6ccccc6)n4)Sc4ccccc4C54c5ccccc5Oc5ccccc54)cc3)n2)cc1 ; N#Cc1cc(-c2nc(-c3ccc(-c4ccccc4)cc3)nc(-c3ccc4c(c3)Sc3ccccc3C43c4ccccc4N(c4ccccn4)c4ccccc43)n2)c2c(c1)C1(CCCCC1)c1ccccc1-2]

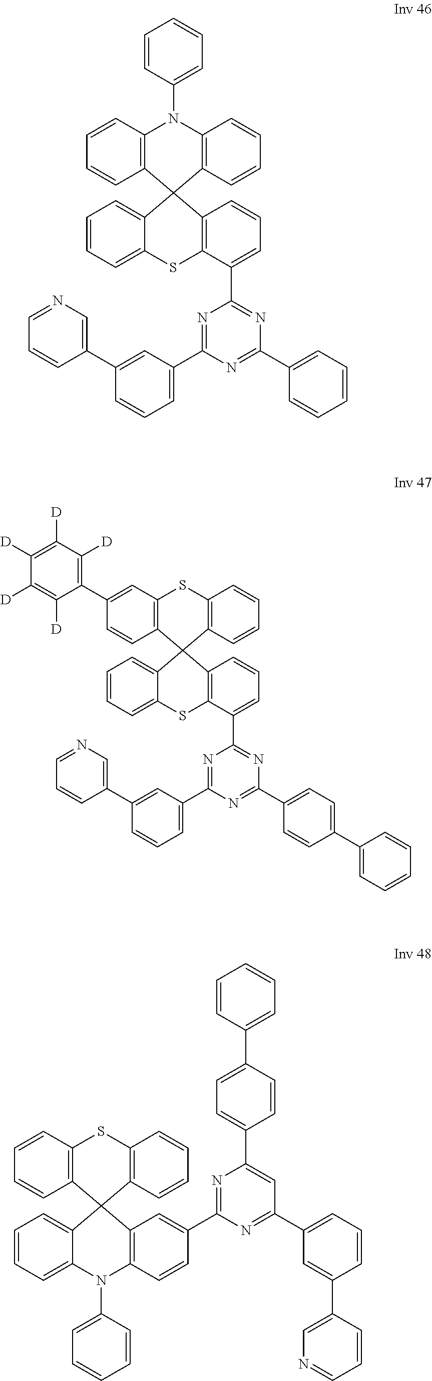 [SMILES: c1ccc(-c2nc(-c3cccc(-c4cccnc4)c3)nc(-c3cccc4c3Sc3ccccc3C43c4ccccc4N(c4ccccc4)c4ccccc43)n2)cc1 ; [2H]c1c([2H])c([2H])c(-c2ccc3c(c2)Sc2ccccc2C32c3ccccc3Sc3c(-c4nc(-c5ccc(-c6ccccc6)cc5)nc(-c5cccc(-c6cccnc6)c5)n4)cccc32)c([2H])c1[2H] ; c1ccc(-c2ccc(-c3cc(-c4cccc(-c5cccnc5)c4)nc(-c4ccc5c(c4)C4(c6ccccc6Sc6ccccc64)c4ccccc4N5c4ccccc4)n3)cc2)cc1]

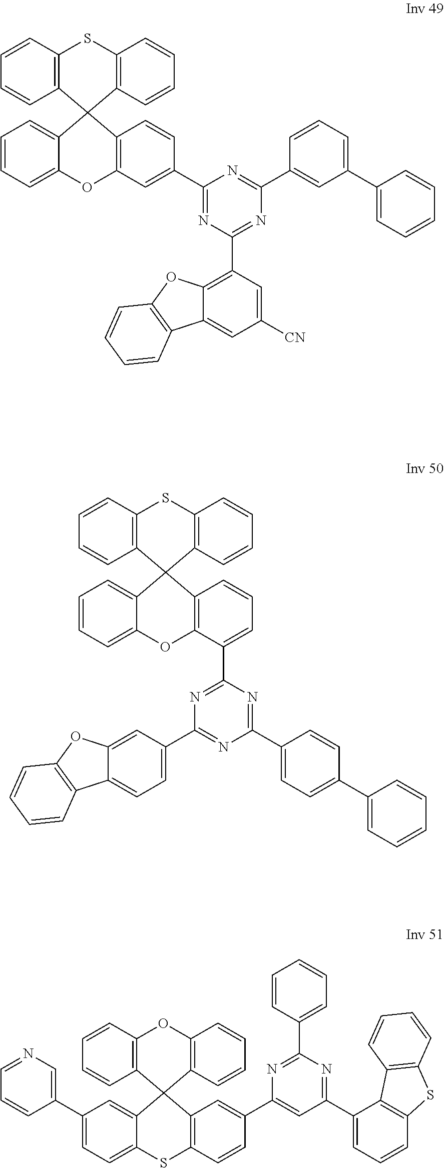 [SMILES: N#Cc1cc(-c2nc(-c3cccc(-c4ccccc4)c3)nc(-c3ccc4c(c3)Oc3ccccc3C43c4ccccc4Sc4ccccc43)n2)c2oc3ccccc3c2c1 ; c1ccc(-c2ccc(-c3nc(-c4ccc5c(c4)oc4ccccc45)nc(-c4cccc5c4Oc4ccccc4C54c5ccccc5Sc5ccccc54)n3)cc2)cc1 ; c1ccc(-c2nc(-c3ccc4c(c3)C3(c5ccccc5Oc5ccccc53)c3cc(-c5cccnc5)ccc3S4)cc(-c3cccc4sc5ccccc5c34)n2)cc1]

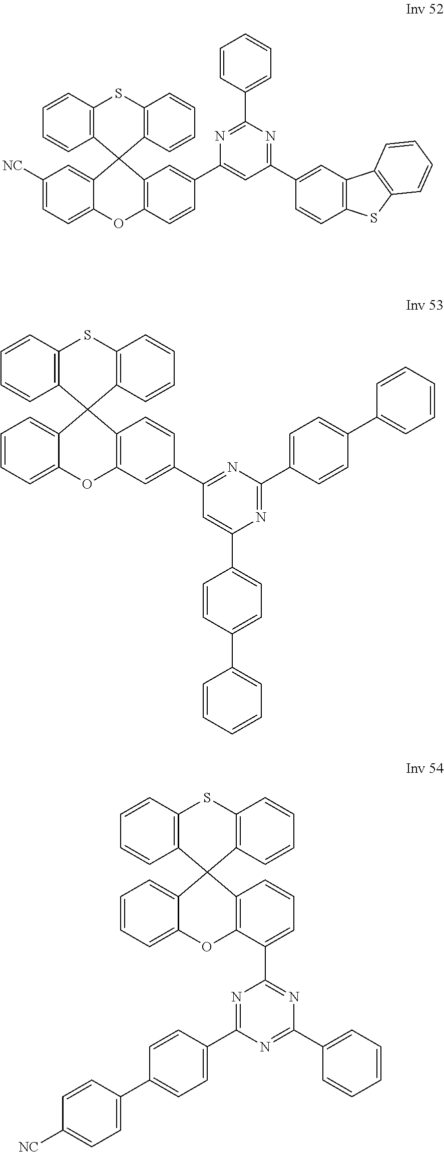 [SMILES: N#Cc1ccc2c(c1)C1(c3cc(-c4cc(-c5ccc6sc7ccccc7c6c5)nc(-c5ccccc5)n4)ccc3O2)c2ccccc2Sc2ccccc21 ; c1ccc(-c2ccc(-c3cc(-c4ccc5c(c4)Oc4ccccc4C54c5ccccc5Sc5ccccc54)nc(-c4ccc(-c5ccccc5)cc4)n3)cc2)cc1 ; N#Cc1ccc(-c2ccc(-c3nc(-c4ccccc4)nc(-c4cccc5c4Oc4ccccc4C54c5ccccc5Sc5ccccc54)n3)cc2)cc1]

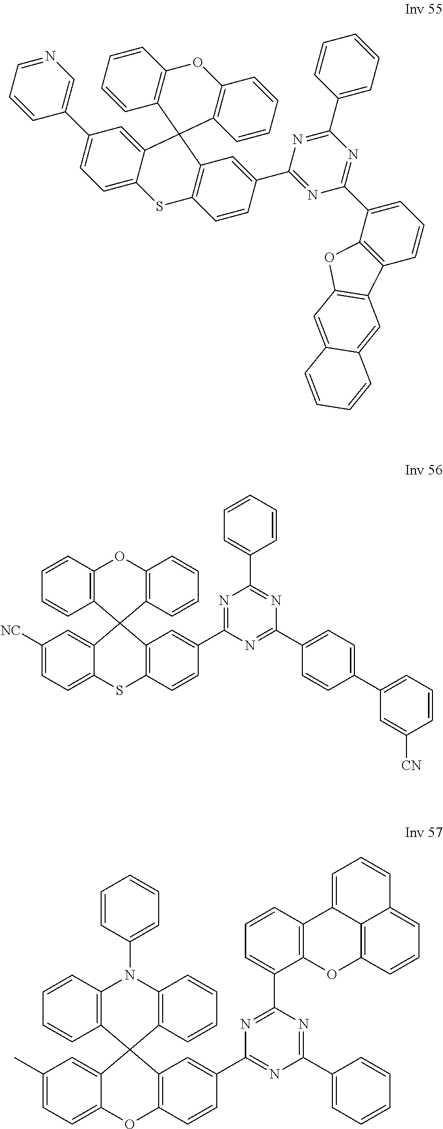 [SMILES: c1ccc(-c2nc(-c3ccc4c(c3)C3(c5ccccc5Oc5ccccc53)c3cc(-c5cccnc5)ccc3S4)nc(-c3cccc4c3oc3cc5ccccc5cc34)n2)cc1 ; N#Cc1cccc(-c2ccc(-c3nc(-c4ccccc4)nc(-c4ccc5c(c4)C4(c6ccccc6Oc6ccccc64)c4cc(C#N)ccc4S5)n3)cc2)c1 ; Cc1ccc2c(c1)C1(c3cc(-c4nc(-c5ccccc5)nc(-c5cccc6c5Oc5cccc7cccc-6c57)n4)ccc3O2)c2ccccc2N(c2ccccc2)c2ccccc21]

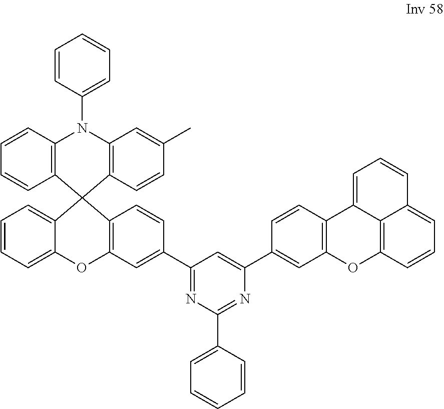 [SMILES: Cc1ccc2c(c1)N(c1ccccc1)c1ccccc1C21c2ccccc2Oc2cc(-c3cc(-c4ccc5c(c4)Oc4cccc6cccc-5c46)nc(-c4ccccc4)n3)ccc21]

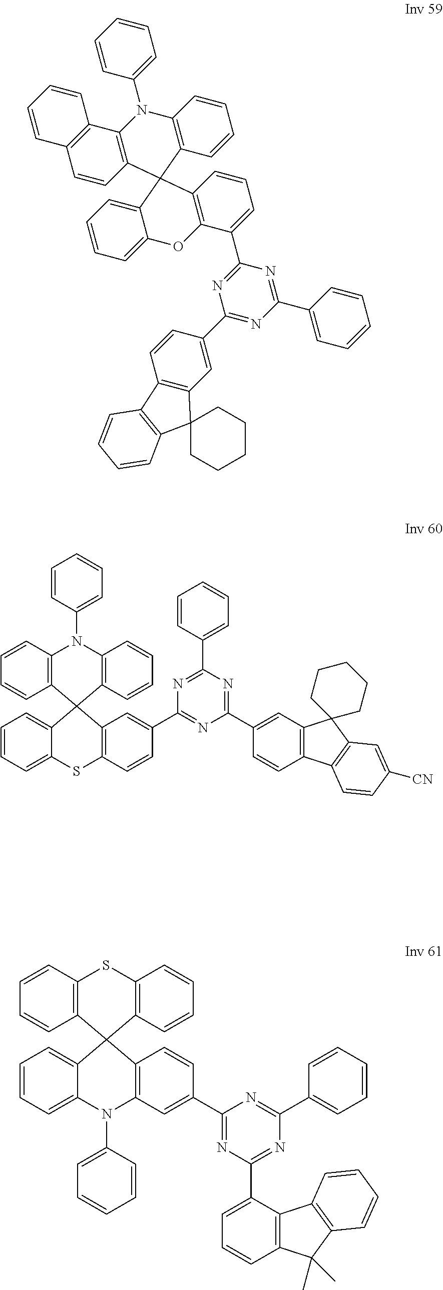 [SMILES: c1ccc(-c2nc(-c3ccc4c(c3)C3(CCCCC3)c3ccccc3-4)nc(-c3cccc4c3Oc3ccccc3C43c4ccccc4N(c4ccccc4)c4c3ccc3ccccc43)n2)cc1 ; N#Cc1ccc2c(c1)C1(CCCCC1)c1cc(-c3nc(-c4ccccc4)nc(-c4ccc5c(c4)C4(c6ccccc6S5)c5ccccc5N(c5ccccc5)c5ccccc54)n3)ccc1-2 ; CC1(C)c2ccccc2-c2c(-c3nc(-c4ccccc4)nc(-c4ccc5c(c4)N(c4ccccc4)c4ccccc4C54c5ccccc5Sc5ccccc54)n3)cccc21]

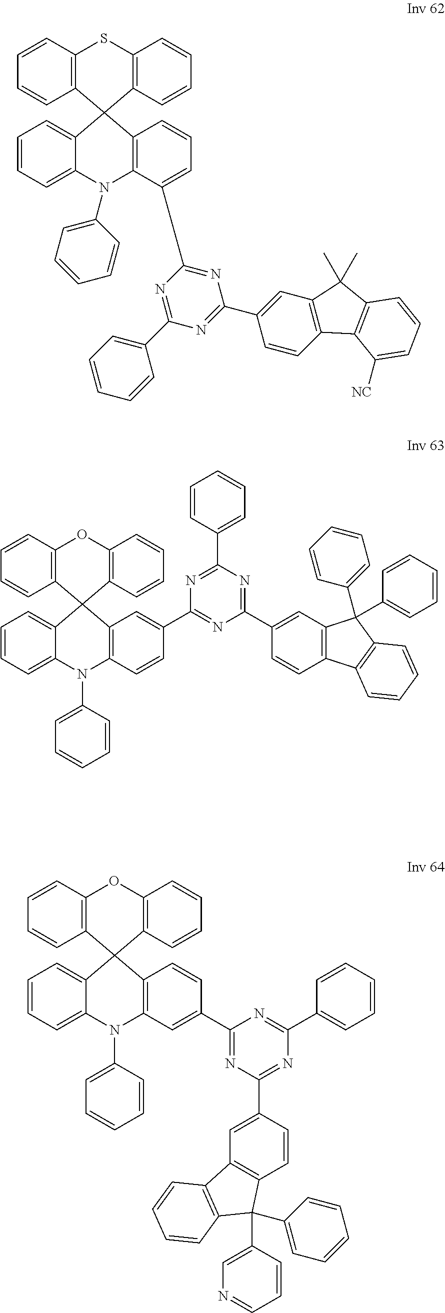 [SMILES: CC1(C)c2cc(-c3nc(-c4ccccc4)nc(-c4cccc5c4N(c4ccccc4)c4ccccc4C54c5ccccc5Sc5ccccc54)n3)ccc2-c2c(C#N)cccc21 ; c1ccc(-c2nc(-c3ccc4c(c3)C(c3ccccc3)(c3ccccc3)c3ccccc3-4)nc(-c3ccc4c(c3)C3(c5ccccc5Oc5ccccc53)c3ccccc3N4c3ccccc3)n2)cc1 ; c1ccc(-c2nc(-c3ccc4c(c3)-c3ccccc3C4(c3ccccc3)c3cccnc3)nc(-c3ccc4c(c3)N(c3ccccc3)c3ccccc3C43c4ccccc4Oc4ccccc43)n2)cc1]

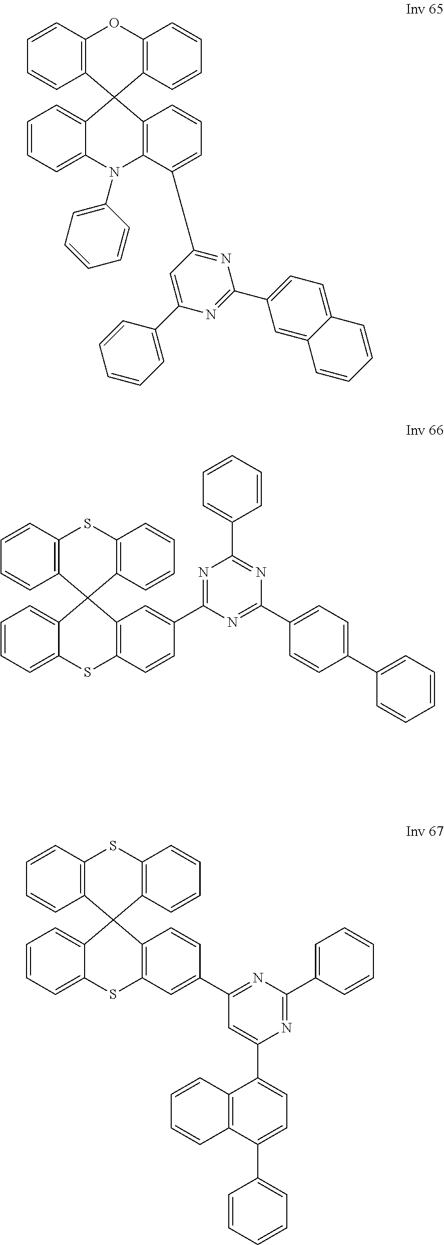 [SMILES: c1ccc(-c2cc(-c3cccc4c3N(c3ccccc3)c3ccccc3C43c4ccccc4Oc4ccccc43)nc(-c3ccc4ccccc4c3)n2)cc1 ; c1ccc(-c2ccc(-c3nc(-c4ccccc4)nc(-c4ccc5c(c4)C4(c6ccccc6Sc6ccccc64)c4ccccc4S5)n3)cc2)cc1 ; c1ccc(-c2nc(-c3ccc4c(c3)Sc3ccccc3C43c4ccccc4Sc4ccccc43)cc(-c3ccc(-c4ccccc4)c4ccccc34)n2)cc1]

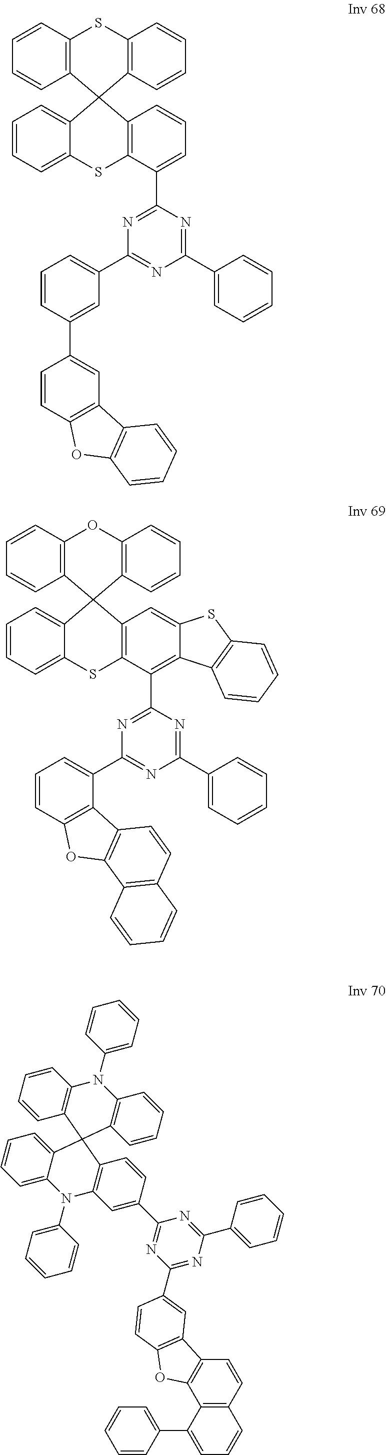

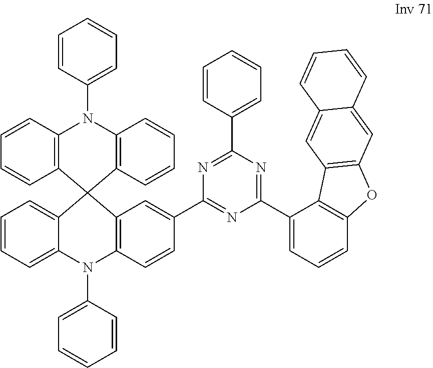 [SMILES: c1ccc(-c2nc(-c3ccc4c(c3)C3(c5ccccc5N(c5ccccc5)c5ccccc53)c3ccccc3N4c3ccccc3)nc(-c3cccc4oc5cc6ccccc6cc5c34)n2)cc1]

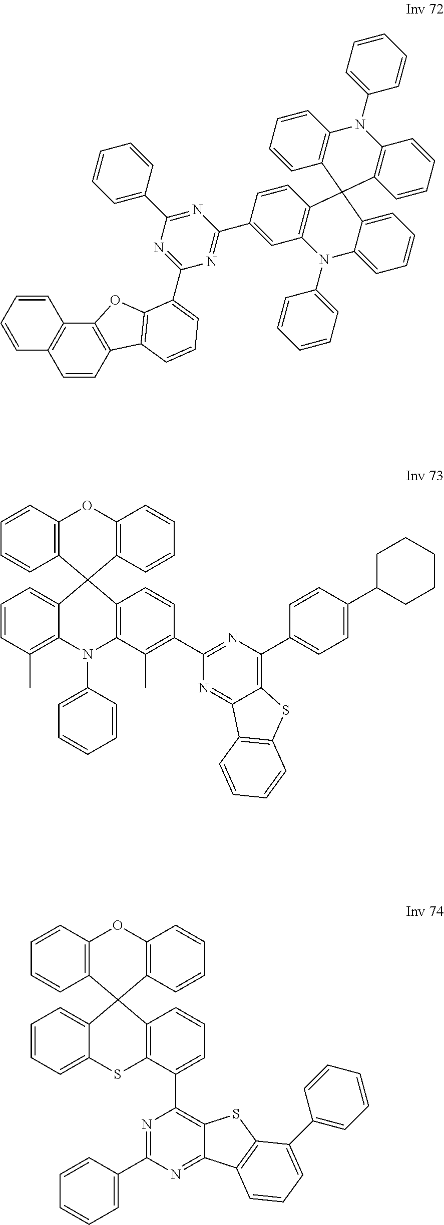 [SMILES: c1ccc(-c2nc(-c3ccc4c(c3)N(c3ccccc3)c3ccccc3C43c4ccccc4N(c4ccccc4)c4ccccc43)nc(-c3cccc4c3oc3c5ccccc5ccc43)n2)cc1 ; Cc1cccc2c1N(c1ccccc1)c1c(ccc(-c3nc(-c4ccc(C5CCCCC5)cc4)c4sc5ccccc5c4n3)c1C)C21c2ccccc2Oc2ccccc21 ; c1ccc(-c2nc(-c3cccc4c3Sc3ccccc3C43c4ccccc4Oc4ccccc43)c3sc4c(-c5ccccc5)cccc4c3n2)cc1]

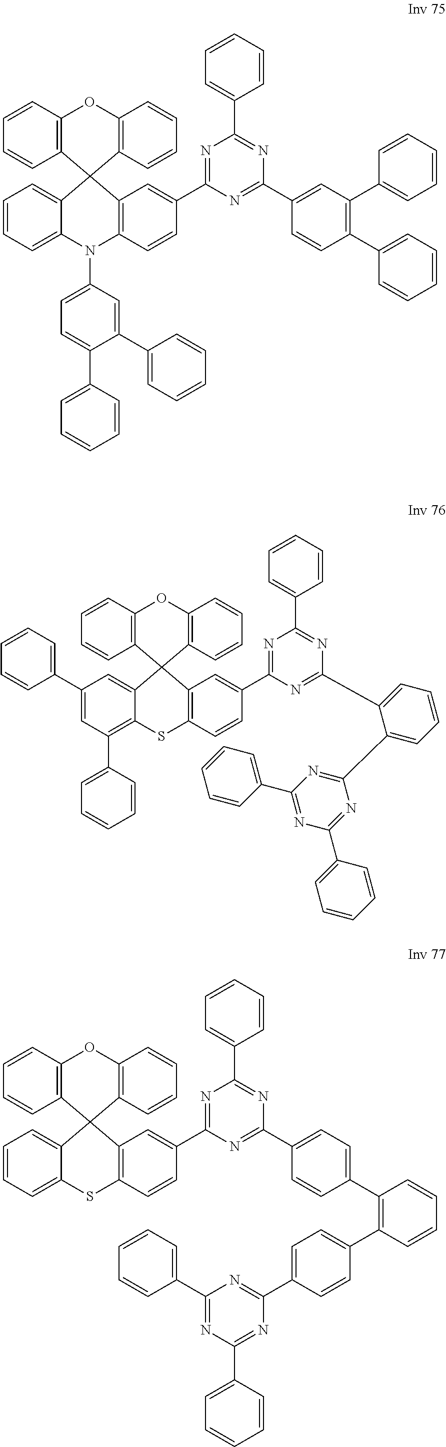 [SMILES: c1ccc(-c2nc(-c3ccc(-c4ccccc4)c(-c4ccccc4)c3)nc(-c3ccc4c(c3)C3(c5ccccc5Oc5ccccc53)c3ccccc3N4c3ccc(-c4ccccc4)c(-c4ccccc4)c3)n2)cc1 ; c1ccc(-c2cc(-c3ccccc3)c3c(c2)C2(c4ccccc4Oc4ccccc42)c2cc(-c4nc(-c5ccccc5)nc(-c5ccccc5-c5nc(-c6ccccc6)nc(-c6ccccc6)n5)n4)ccc2S3)cc1 ; c1ccc(-c2nc(-c3ccccc3)nc(-c3ccc(-c4ccccc4-c4ccc(-c5nc(-c6ccccc6)nc(-c6ccc7c(c6)C6(c8ccccc8Oc8ccccc86)c6ccccc6S7)n5)cc4)cc3)n2)cc1]

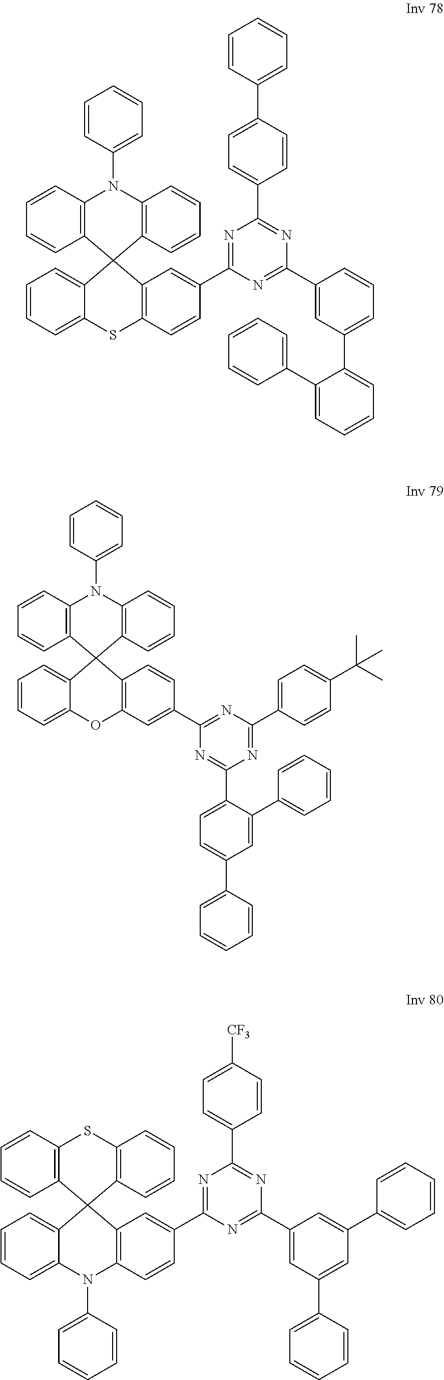 [SMILES: c1ccc(-c2ccc(-c3nc(-c4cccc(-c5ccccc5-c5ccccc5)c4)nc(-c4ccc5c(c4)C4(c6ccccc6S5)c5ccccc5N(c5ccccc5)c5ccccc54)n3)cc2)cc1 ; CC(C)(C)c1ccc(-c2nc(-c3ccc4c(c3)Oc3ccccc3C43c4ccccc4N(c4ccccc4)c4ccccc43)nc(-c3ccc(-c4ccccc4)cc3-c3ccccc3)n2)cc1 ; FC(F)(F)c1ccc(-c2nc(-c3cc(-c4ccccc4)cc(-c4ccccc4)c3)nc(-c3ccc4c(c3)C3(c5ccccc5Sc5ccccc53)c3ccccc3N4c3ccccc3)n2)cc1]

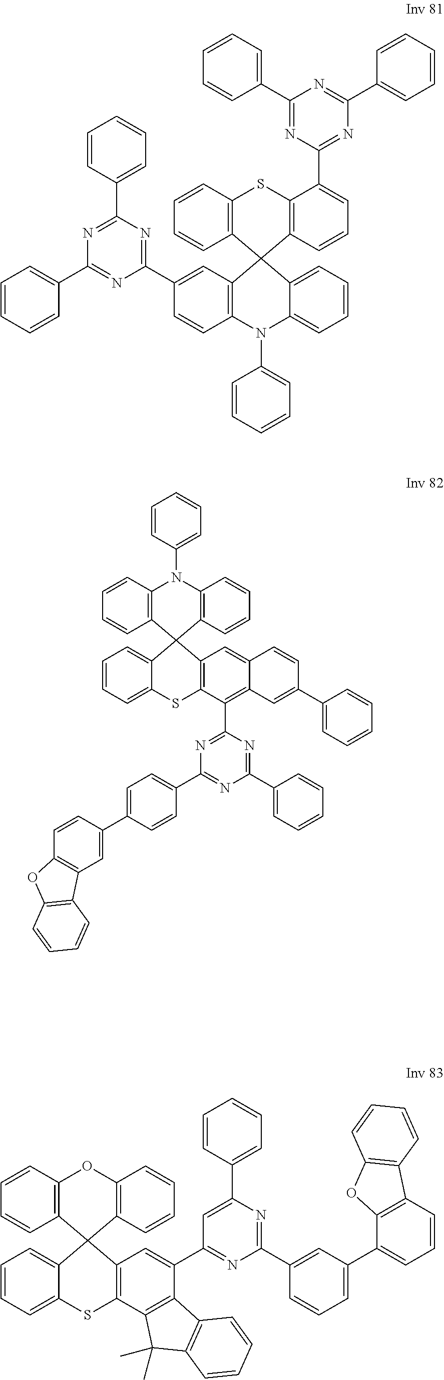 [SMILES: c1ccc(-c2nc(-c3ccccc3)nc(-c3ccc4c(c3)C3(c5ccccc5Sc5c(-c6nc(-c7ccccc7)nc(-c7ccccc7)n6)cccc53)c3ccccc3N4c3ccccc3)n2)cc1 ; c1ccc(-c2ccc3cc4c(c(-c5nc(-c6ccccc6)nc(-c6ccc(-c7ccc8oc9ccccc9c8c7)cc6)n5)c3c2)Sc2ccccc2C42c3ccccc3N(c3ccccc3)c3ccccc32)cc1 ; CC1(C)c2ccccc2-c2c(-c3cc(-c4ccccc4)nc(-c4cccc(-c5cccc6c5oc5ccccc56)c4)n3)cc3c(c21)Sc1ccccc1C31c2ccccc2Oc2ccccc21]

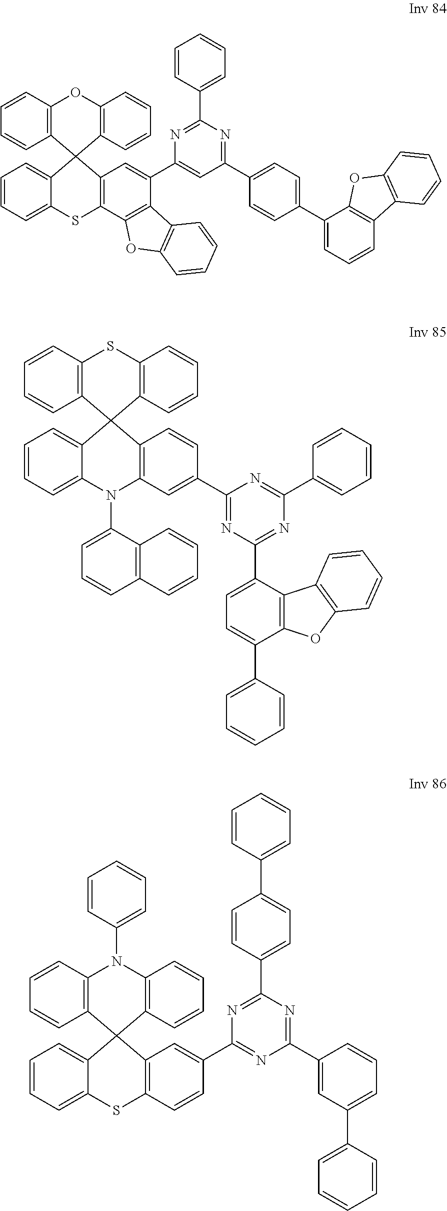 [SMILES: c1ccc(-c2nc(-c3ccc(-c4cccc5c4oc4ccccc45)cc3)cc(-c3cc4c(c5oc6ccccc6c35)Sc3ccccc3C43c4ccccc4Oc4ccccc43)n2)cc1 ; c1ccc(-c2nc(-c3ccc4c(c3)N(c3cccc5ccccc35)c3ccccc3C43c4ccccc4Sc4ccccc43)nc(-c3ccc(-c4ccccc4)c4oc5ccccc5c34)n2)cc1 ; c1ccc(-c2ccc(-c3nc(-c4cccc(-c5ccccc5)c4)nc(-c4ccc5c(c4)C4(c6ccccc6S5)c5ccccc5N(c5ccccc5)c5ccccc54)n3)cc2)cc1]

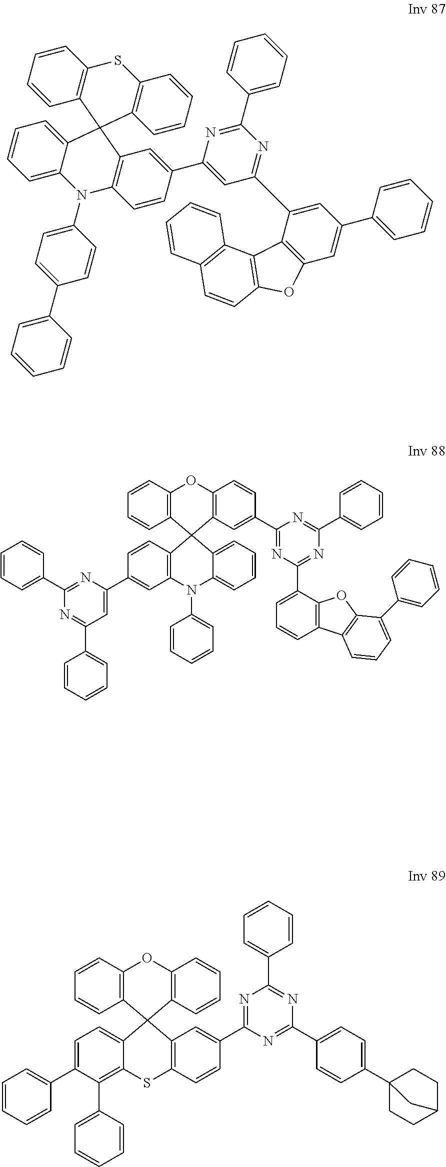 [SMILES: c1ccc(-c2ccc(N3c4ccccc4C4(c5ccccc5Sc5ccccc54)c4cc(-c5cc(-c6cc(-c7ccccc7)cc7oc8ccc9ccccc9c8c67)nc(-c6ccccc6)n5)ccc43)cc2)cc1 ; c1ccc(-c2cc(-c3ccc4c(c3)N(c3ccccc3)c3ccccc3C43c4ccccc4Oc4ccc(-c5nc(-c6ccccc6)nc(-c6cccc7c6oc6c(-c8ccccc8)cccc67)n5)cc43)nc(-c3ccccc3)n2)cc1 ; c1ccc(-c2nc(-c3ccc(C45CCC(CC4)C5)cc3)nc(-c3ccc4c(c3)C3(c5ccccc5Oc5ccccc53)c3ccc(-c5ccccc5)c(-c5ccccc5)c3S4)n2)cc1]

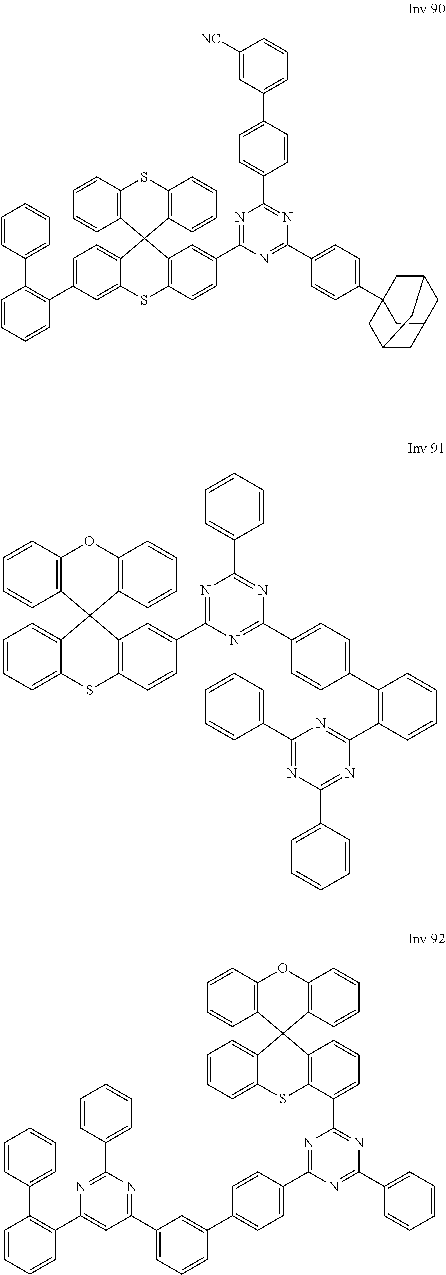 [SMILES: N#Cc1cccc(-c2ccc(-c3nc(-c4ccc(C56CC7CC(CC(C7)C5)C6)cc4)nc(-c4ccc5c(c4)C4(c6ccccc6Sc6ccccc64)c4ccc(-c6ccccc6-c6ccccc6)cc4S5)n3)cc2)c1 ; c1ccc(-c2nc(-c3ccc(-c4ccccc4-c4nc(-c5ccccc5)nc(-c5ccccc5)n4)cc3)nc(-c3ccc4c(c3)C3(c5ccccc5Oc5ccccc53)c3ccccc3S4)n2)cc1 ; c1ccc(-c2nc(-c3cccc(-c4ccc(-c5nc(-c6ccccc6)nc(-c6cccc7c6Sc6ccccc6C76c7ccccc7Oc7ccccc76)n5)cc4)c3)cc(-c3ccccc3-c3ccccc3)n2)cc1]

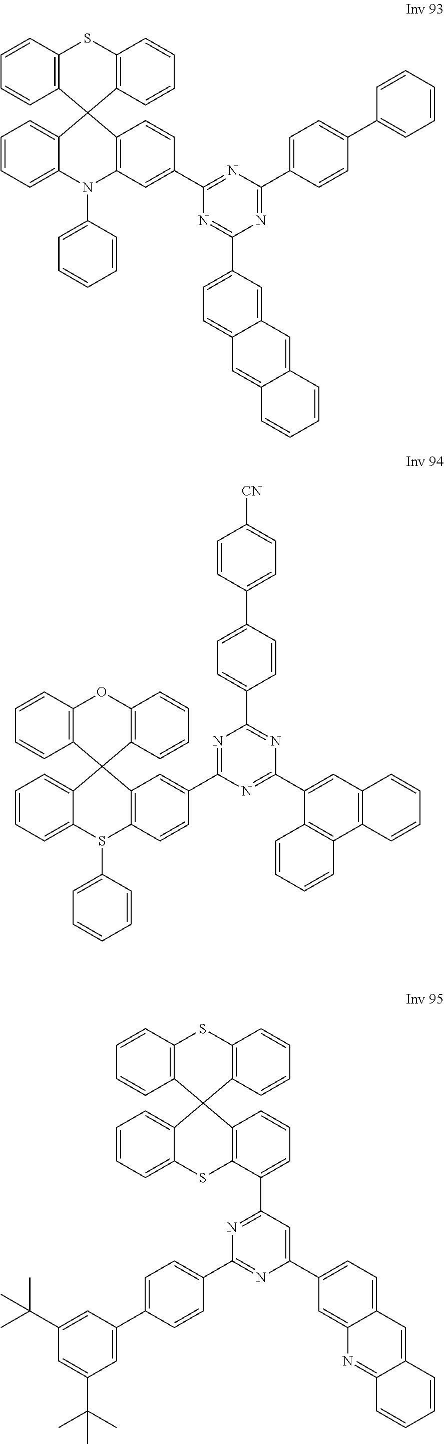

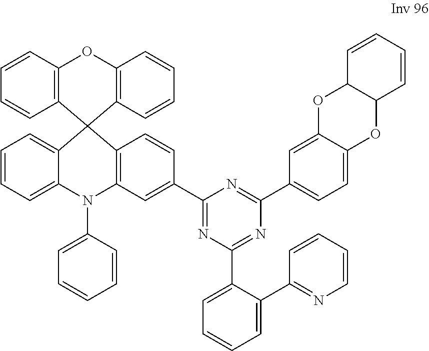 [SMILES: C1=CC2Oc3ccc(-c4nc(-c5ccc6c(c5)N(c5ccccc5)c5ccccc5C65c6ccccc6Oc6ccccc65)nc(-c5ccccc5-c5ccccn5)n4)cc3OC2C=C1]

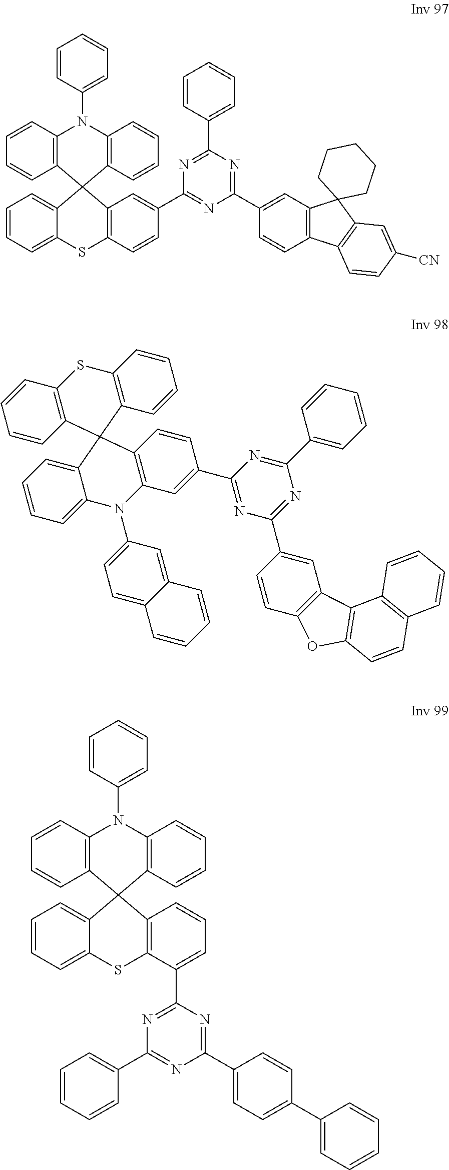 [SMILES: N#Cc1ccc2c(c1)C1(CCCCC1)c1cc(-c3nc(-c4ccccc4)nc(-c4ccc5c(c4)C4(c6ccccc6S5)c5ccccc5N(c5ccccc5)c5ccccc54)n3)ccc1-2 ; c1ccc(-c2nc(-c3ccc4c(c3)N(c3ccc5ccccc5c3)c3ccccc3C43c4ccccc4Sc4ccccc43)nc(-c3ccc4oc5ccc6ccccc6c5c4c3)n2)cc1 ; c1ccc(-c2ccc(-c3nc(-c4ccccc4)nc(-c4cccc5c4Sc4ccccc4C54c5ccccc5N(c5ccccc5)c5ccccc54)n3)cc2)cc1]

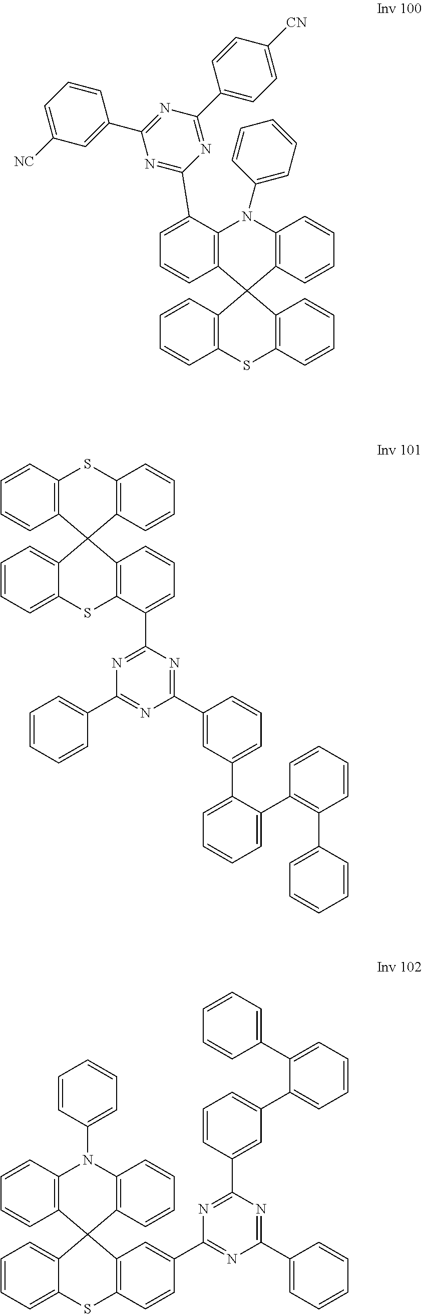 [SMILES: N#Cc1ccc(-c2nc(-c3cccc(C#N)c3)nc(-c3cccc4c3N(c3ccccc3)c3ccccc3C43c4ccccc4Sc4ccccc43)n2)cc1 ; c1ccc(-c2nc(-c3cccc(-c4ccccc4-c4ccccc4-c4ccccc4)c3)nc(-c3cccc4c3Sc3ccccc3C43c4ccccc4Sc4ccccc43)n2)cc1 ; c1ccc(-c2nc(-c3cccc(-c4ccccc4-c4ccccc4)c3)nc(-c3ccc4c(c3)C3(c5ccccc5S4)c4ccccc4N(c4ccccc4)c4ccccc43)n2)cc1]

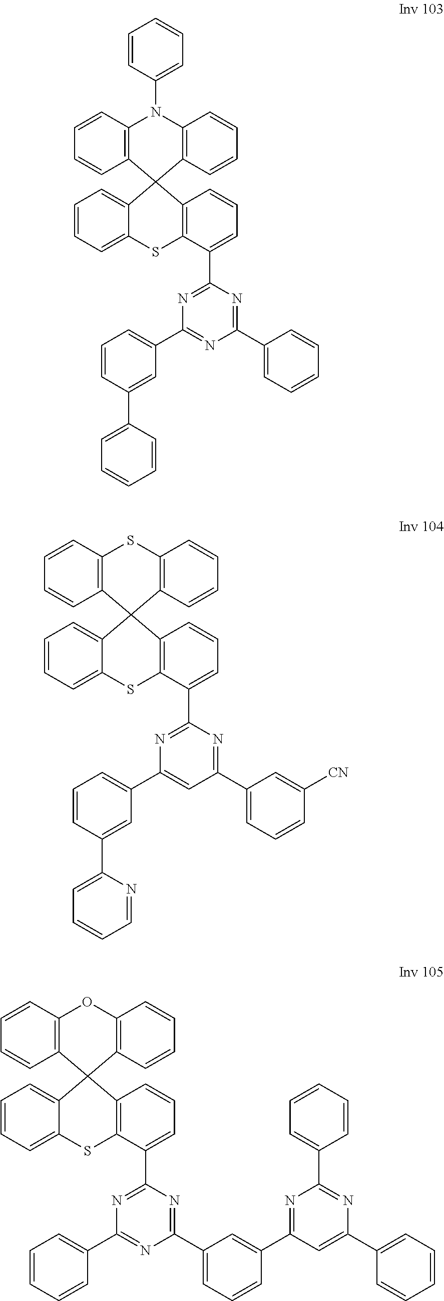 [SMILES: c1ccc(-c2cccc(-c3nc(-c4ccccc4)nc(-c4cccc5c4Sc4ccccc4C54c5ccccc5N(c5ccccc5)c5ccccc54)n3)c2)cc1 ; N#Cc1cccc(-c2cc(-c3cccc(-c4ccccn4)c3)nc(-c3cccc4c3Sc3ccccc3C43c4ccccc4Sc4ccccc43)n2)c1 ; c1ccc(-c2cc(-c3cccc(-c4nc(-c5ccccc5)nc(-c5cccc6c5Sc5ccccc5C65c6ccccc6Oc6ccccc65)n4)c3)nc(-c3ccccc3)n2)cc1]

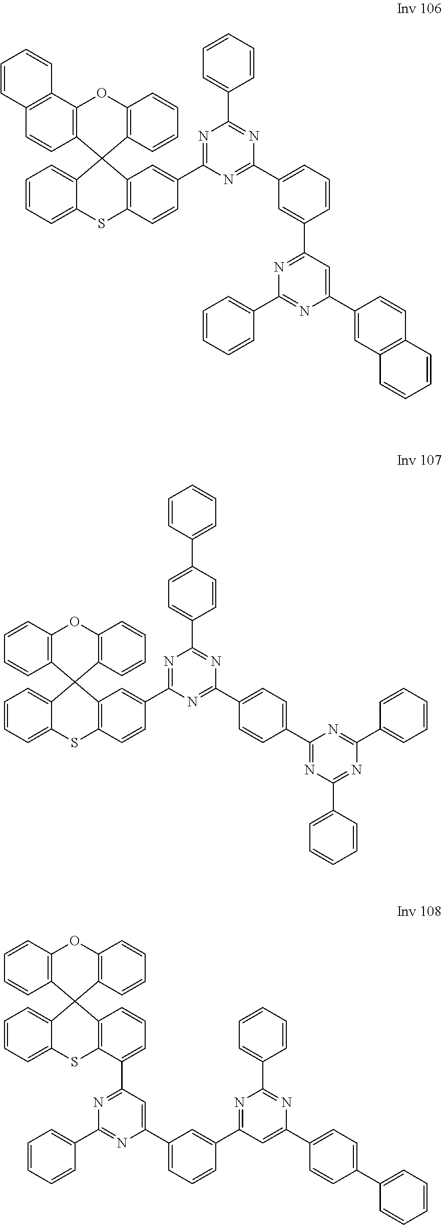 [SMILES: c1ccc(-c2nc(-c3cccc(-c4nc(-c5ccccc5)nc(-c5ccc6c(c5)C5(c7ccccc7Oc7c5ccc5ccccc75)c5ccccc5S6)n4)c3)cc(-c3ccc4ccccc4c3)n2)cc1 ; c1ccc(-c2ccc(-c3nc(-c4ccc(-c5nc(-c6ccccc6)nc(-c6ccccc6)n5)cc4)nc(-c4ccc5c(c4)C4(c6ccccc6Oc6ccccc64)c4ccccc4S5)n3)cc2)cc1 ; c1ccc(-c2ccc(-c3cc(-c4cccc(-c5cc(-c6cccc7c6Sc6ccccc6C76c7ccccc7Oc7ccccc76)nc(-c6ccccc6)n5)c4)nc(-c4ccccc4)n3)cc2)cc1]

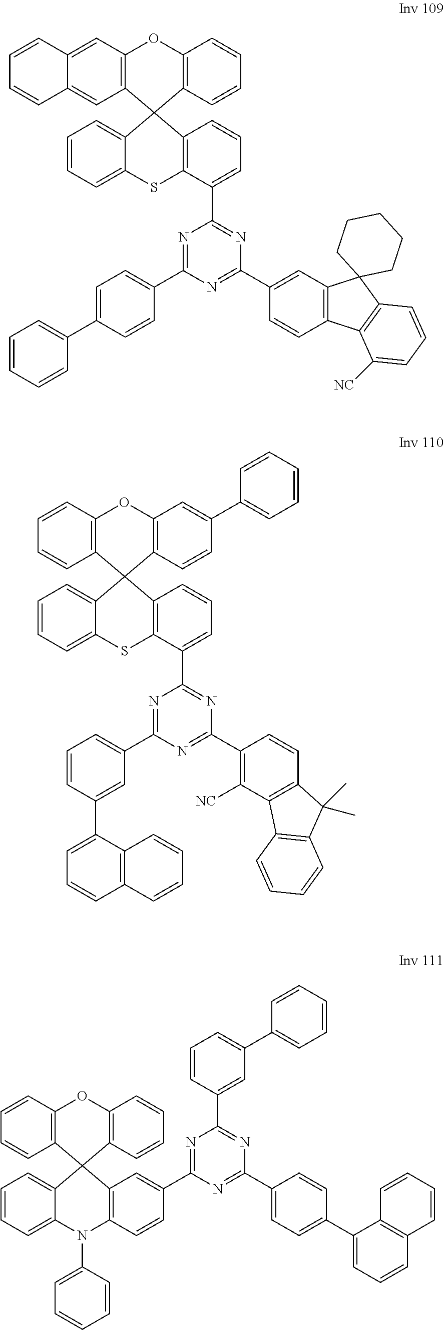 [SMILES: N#Cc1cccc2c1-c1ccc(-c3nc(-c4ccc(-c5ccccc5)cc4)nc(-c4cccc5c4Sc4ccccc4C54c5ccccc5Oc5cc6ccccc6cc54)n3)cc1C21CCCCC1 ; CC1(C)c2ccccc2-c2c1ccc(-c1nc(-c3cccc(-c4cccc5ccccc45)c3)nc(-c3cccc4c3Sc3ccccc3C43c4ccccc4Oc4cc(-c5ccccc5)ccc43)n1)c2C#N ; c1ccc(-c2cccc(-c3nc(-c4ccc(-c5cccc6ccccc56)cc4)nc(-c4ccc5c(c4)C4(c6ccccc6Oc6ccccc64)c4ccccc4N5c4ccccc4)n3)c2)cc1]

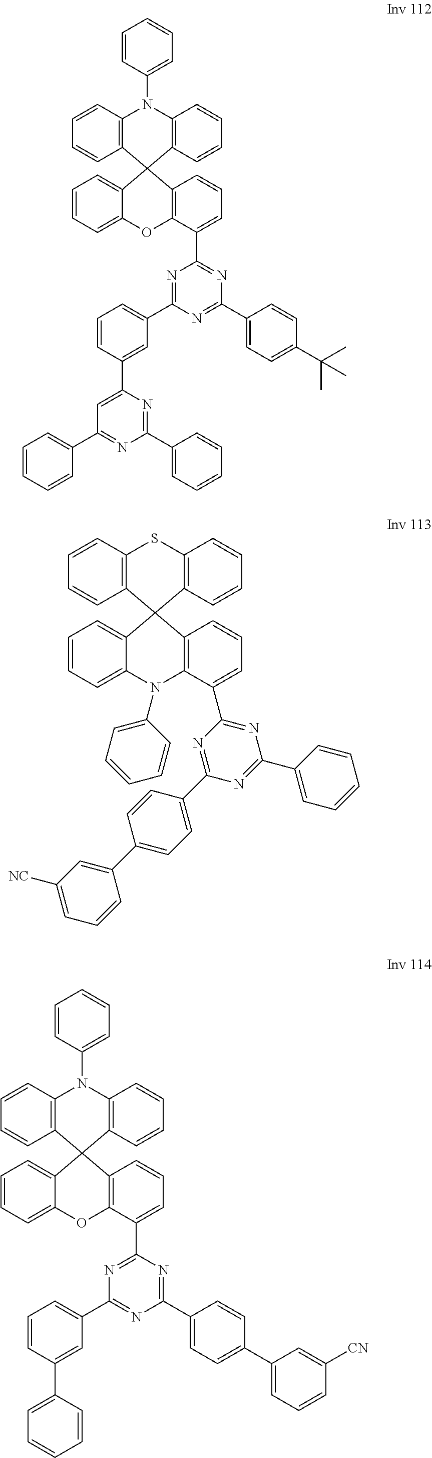 [SMILES: CC(C)(C)c1ccc(-c2nc(-c3cccc(-c4cc(-c5ccccc5)nc(-c5ccccc5)n4)c3)nc(-c3cccc4c3Oc3ccccc3C43c4ccccc4N(c4ccccc4)c4ccccc43)n2)cc1 ; N#Cc1cccc(-c2ccc(-c3nc(-c4ccccc4)nc(-c4cccc5c4N(c4ccccc4)c4ccccc4C54c5ccccc5Sc5ccccc54)n3)cc2)c1 ; N#Cc1cccc(-c2ccc(-c3nc(-c4cccc(-c5ccccc5)c4)nc(-c4cccc5c4Oc4ccccc4C54c5ccccc5N(c5ccccc5)c5ccccc54)n3)cc2)c1]

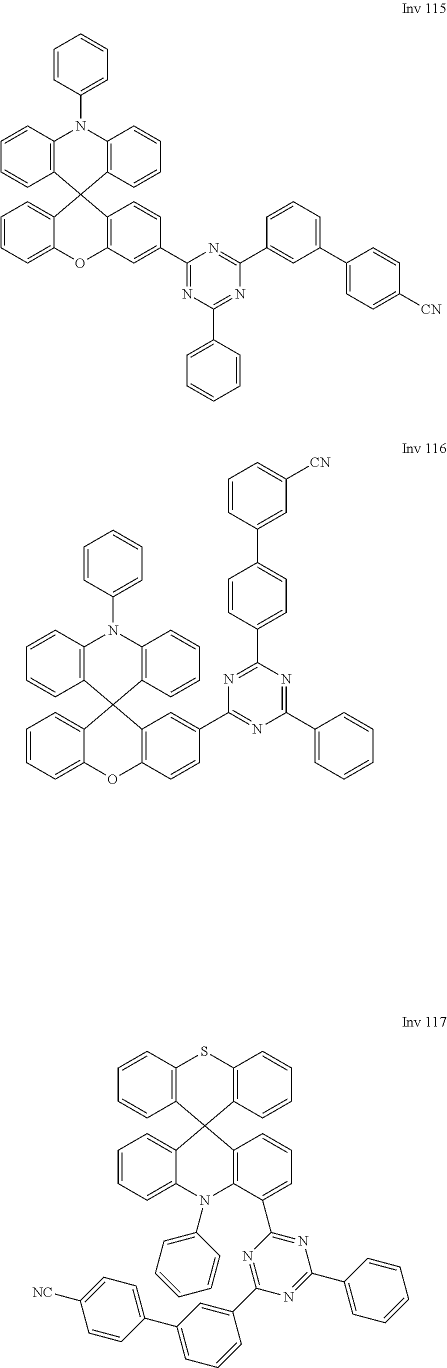 [SMILES: N#Cc1ccc(-c2cccc(-c3nc(-c4ccccc4)nc(-c4ccc5c(c4)Oc4ccccc4C54c5ccccc5N(c5ccccc5)c5ccccc54)n3)c2)cc1 ; N#Cc1cccc(-c2ccc(-c3nc(-c4ccccc4)nc(-c4ccc5c(c4)C4(c6ccccc6O5)c5ccccc5N(c5ccccc5)c5ccccc54)n3)cc2)c1 ; N#Cc1ccc(-c2cccc(-c3nc(-c4ccccc4)nc(-c4cccc5c4N(c4ccccc4)c4ccccc4C54c5ccccc5Sc5ccccc54)n3)c2)cc1]

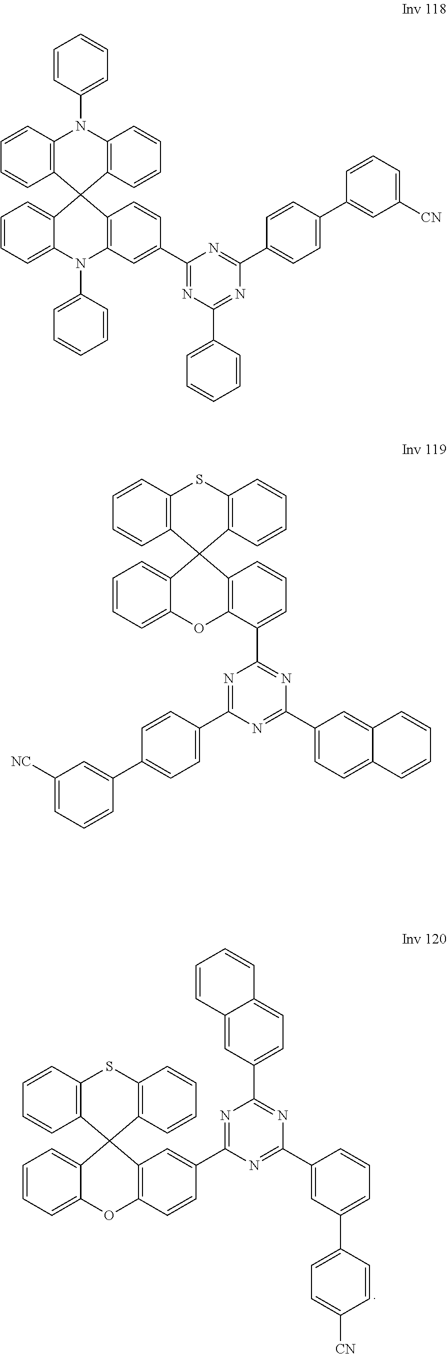 [SMILES: N#Cc1cccc(-c2ccc(-c3nc(-c4ccccc4)nc(-c4ccc5c(c4)N(c4ccccc4)c4ccccc4C54c5ccccc5N(c5ccccc5)c5ccccc54)n3)cc2)c1 ; N#Cc1cccc(-c2ccc(-c3nc(-c4ccc5ccccc5c4)nc(-c4cccc5c4Oc4ccccc4C54c5ccccc5Sc5ccccc54)n3)cc2)c1 ; N#Cc1ccc(-c2cccc(-c3nc(-c4ccc5c(c4)C4(c6ccccc6O5)c5ccccc5Sc5ccccc54)nc(-c4ccc5ccccc5c4)n3)c2)cc1 ; "."]

28. The compound of claim 16, wherein the compound represented by Chemical Formula 1 is used as a material of a light-emitting layer, an electron transport layer, or an auxiliary electron transport layer.

29. An organic electroluminescent device comprising:
an anode;
a cathode; and
one or more organic layers disposed between the anode and the cathode,
wherein at least one of the one or more organic layers comprises a compound represented by the following Chemical Formula 1: 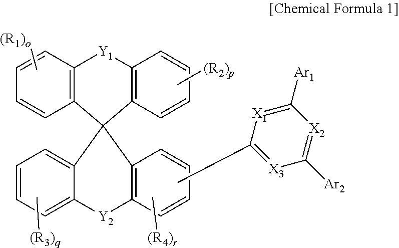
wherein in Chemical Formula 1,
X₁ to X₃ are the same as or different from each other and are each independently C(R₅) or N, provided that at least two of X₁ to X₃ are N;
Y₁ and Y₂ are the same as or different from each other and are each independently selected from the group consisting of O, S, and NR₁₁, provided that the case where both Y₁ and Y₂ are O is excluded;
Ar₁ and Ar₂ are the same as or different from each other and are each independently selected from the group consisting of a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 ring atoms, a C₆-C₆₀ aryl group, a heteroaryl group having 5 to 60 ring atoms, a C₁-C₄₀ alkyloxy group, a C₆-C₆₀ aryloxy group, a C₃-C₄₀ alkylsilyl group, a C₆-C₆₀ arylsilyl group, a C₁-C₄₀ alkylboron group, a C₆-C₆₀ arylboron group, a C₆-C₆₀ arylphosphine group, a C₆-C₆₀ arylphosphine oxide group, and a C₆-C₆₀ arylamine group, or combine with an adjacent group to form a fused ring;
R₁ to R₅ and R₁₁ are the same as or different from one another and are each independently selected from the group consisting of a hydrogen, a deuterium (D), a halogen, a cyano group, a nitro group, a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 ring atoms, a C₆-C₆₀ aryl group, a heteroaryl group having 5 to 60 ring atoms, a C₁-C₄₀ alkyloxy group, a C₆-C₆ aryloxy group, a C₃-C₄₀ alkylsilyl group, a C₆-C₆ arylsilyl group, a C₁-C₄₀ alkylboron group, a C₆-C₆ arylboron group, a C₆-C₆ aryl phosphine group, a C₆-C₆₀ arylphosphine oxide group, and a C₆-C₆₀ arylamine group, or combine with an adjacent group to form a fused ring;
o, p, and q are each independently an integer of 0 to 4, and r is an integer of 0 to 3; and
the alkyl group, alkenyl group, alkynyl group, aryl group, heteroaryl group, aryloxy group, alkyloxy group, cycloalkyl group, heterocycloalkyl group, alkylsilyl group, arylsilyl group, alkylboron group, arylboron group, arylphosphine group, arylphosphine oxide group, and arylamine group of Ar₁ to Ar₂, R₁ to R₅, and R₁ may be each independently substituted with at least one substituent selected from the group consisting of a deuterium (D), a halogen, a cyano group, a nitro group, a C₁ to C₄₀ alkyl group, a C₂ to C₄₀ alkenyl group, a C₂ to C₄₀ alkynyl group, a C₃ to C₆₀ cycloalkyl group, a heterocycloalkyl group having 3 to 40 ring atoms, a C₆ to C₆₀ aryl group, a heteroaryl group having 5 to 60 ring atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, and wherein when the substituent is present in a plural number, they may be the same or different from each other.

30. The organic electroluminescent device of claim 29, wherein the Y₁ and Y₂ containing ring is selected from the group of substituents represented by the following structural formulas: 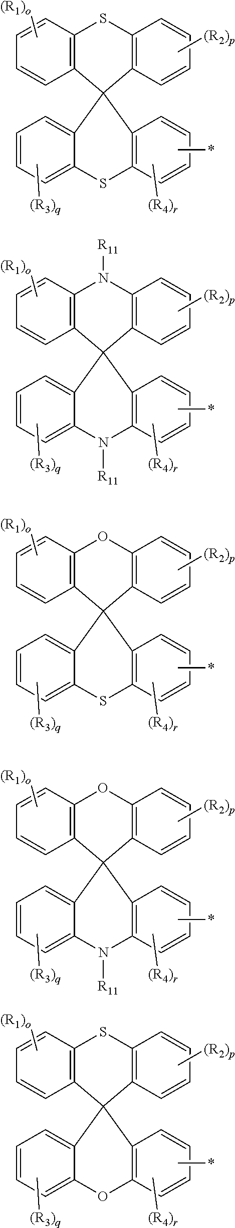 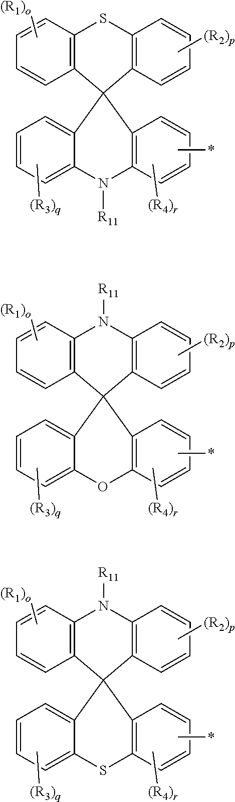
wherein in the formulas,
* indicates a site where a bond with Chemical Formula 1 is made,
R₁₁ is selected from the group consisting of a hydrogen, a deuterium (D), a C₁ to C₄₀ alkyl group, a C₆ to C₃₀ aryl group, and a heteroaryl group having 5 to 30 nuclear atoms,
R₁ to R₄, o, p, q and r are the same as defined in claim 29.

31. The organic electroluminescent device of claim 29, wherein the X-containing ring is selected from the group of substituents represented by the following structural formulas: 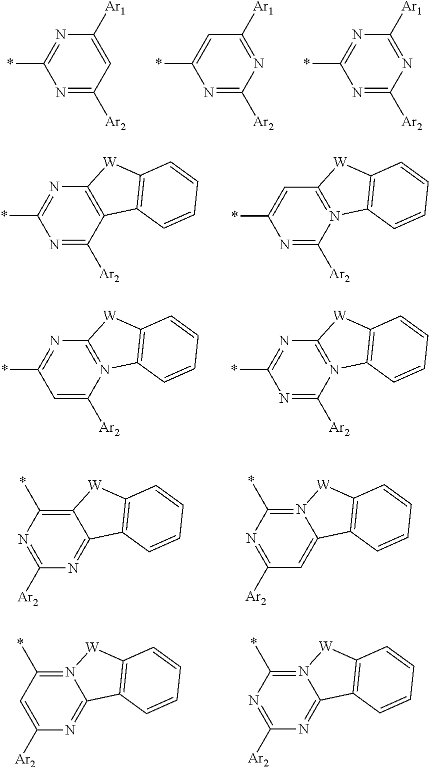
wherein in the formulas,
* indicates a site where a bond with Chemical Formula 1 is made,
W is 0 or S, and
Ar₁ and Ar₂ are the same as defined in claim 29.

32. The organic electroluminescent device of claim 29, wherein the compound represented by Chemical Formula 1 is a compound represented by any one of Chemical Formula 2 to Chemical Formula 9 below: 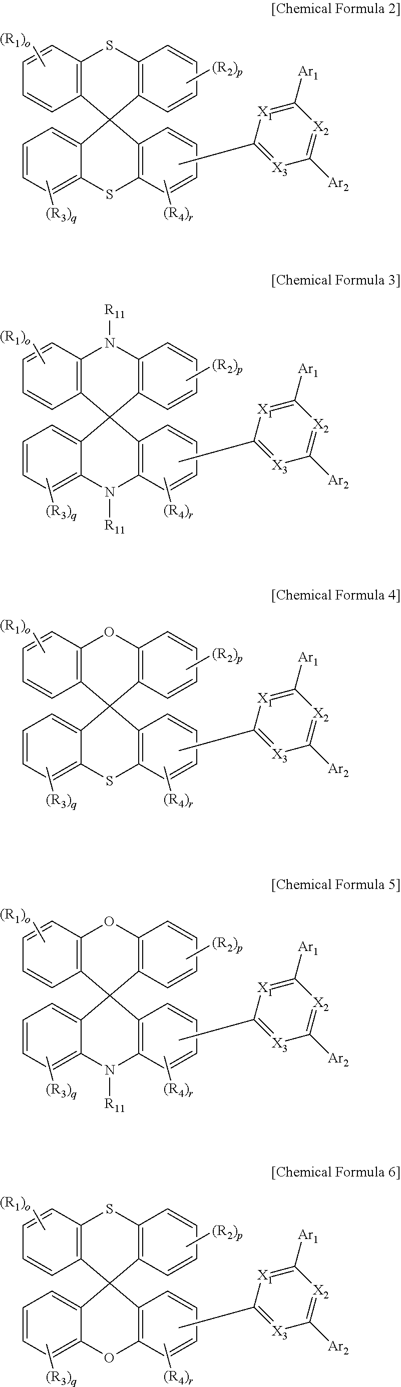 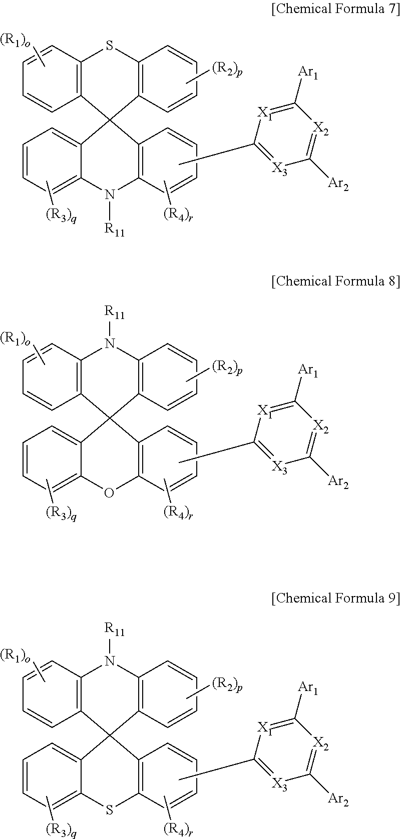
wherein in the formulas,
X₁ to X₃, Ar₁ to Ar₂, R₁ to R₄, R₁₁, o, p, q and r are the same as defined in claim 29.

33. The organic electroluminescent device of claim 29, wherein the compound represented by Chemical Formula 1 is a compound represented by any one of Chemical Formula 10 to Chemical Formula 20 below: 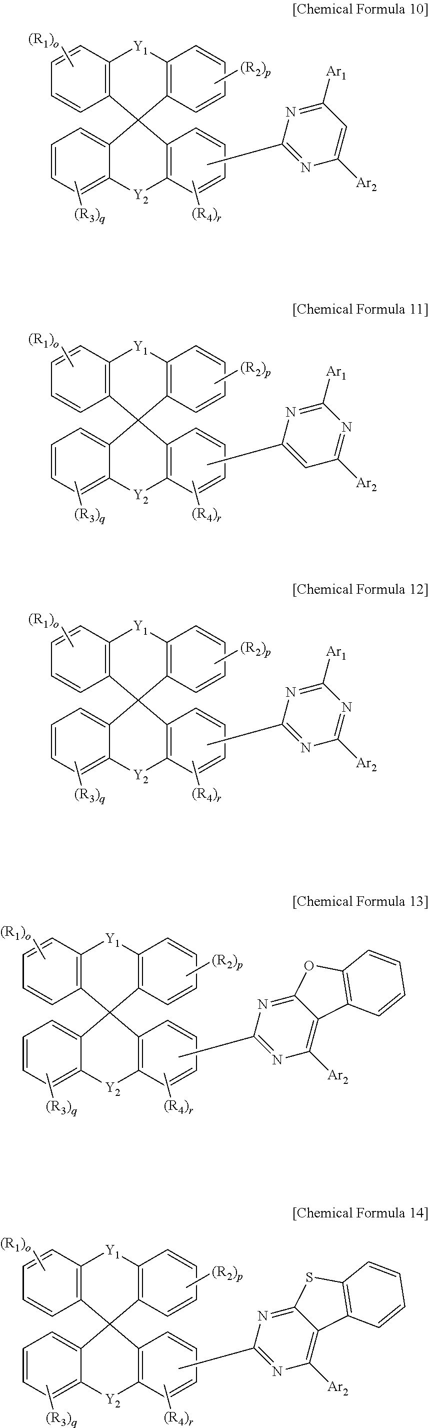 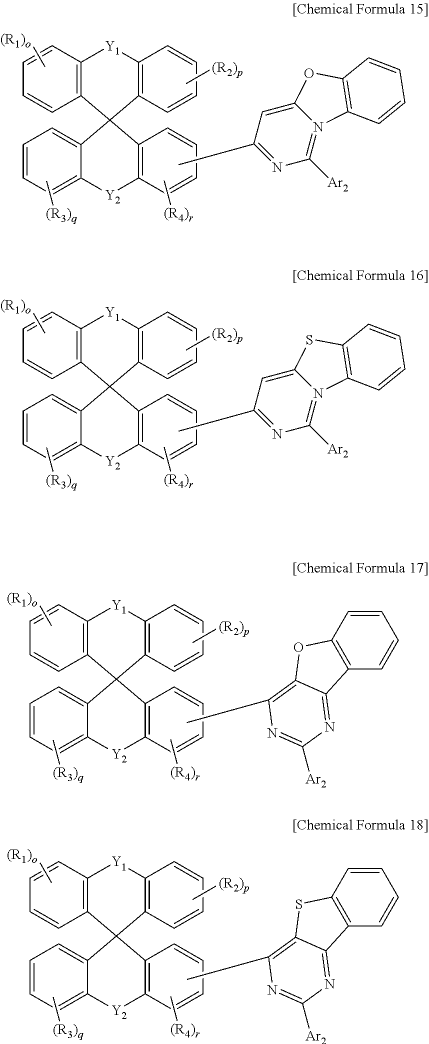 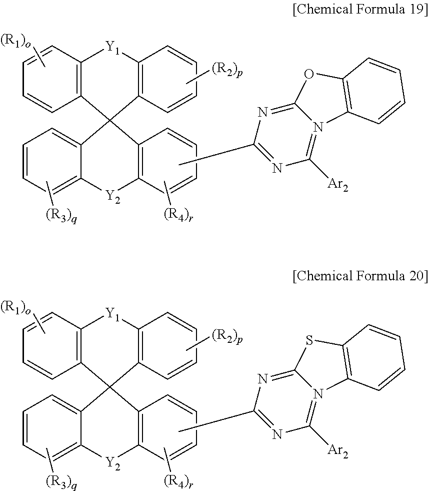
wherein in the formulas,
Y₁ to Y₂, Ar₁ to Ar₂, R₁ to R₄, o, p, q and r are the same as defined in claim 29.

34. The organic electroluminescent device of claim 29, wherein the compound represented by Chemical Formula 1 is a compound represented by any one of Chemical Formula 21 to Chemical Formula 28 below: 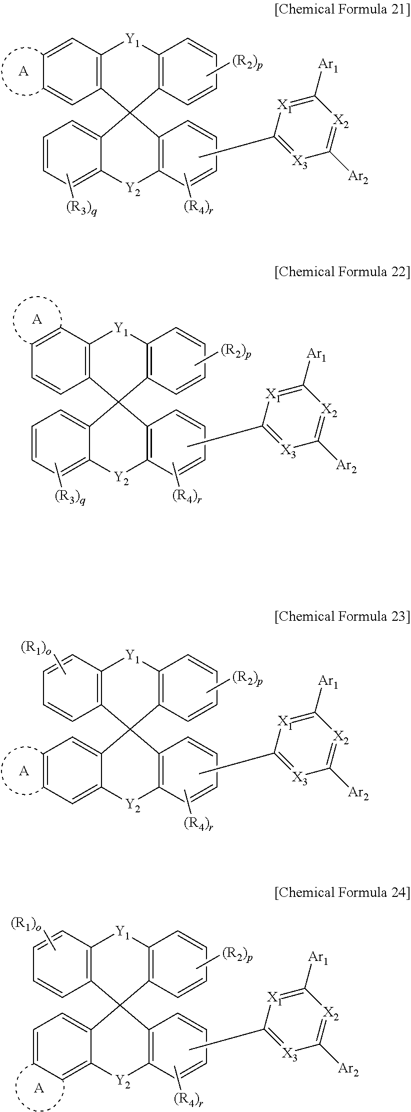 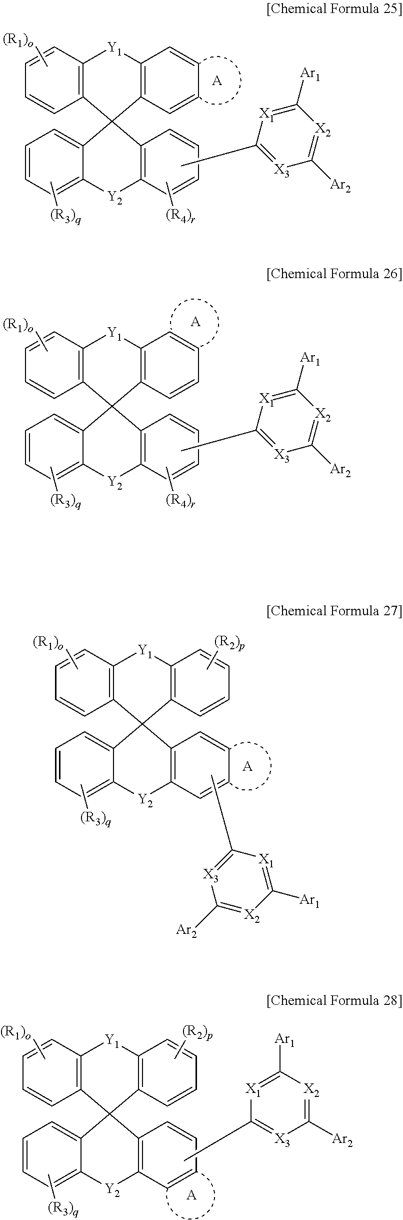
wherein in the formulas,
Ring A is a monocyclic or polycyclic alicyclic ring, a monocyclic or polycyclic heteroalicyclic ring, a monocyclic or polycyclic aromatic ring, or a monocyclic or polycyclic heteroaromatic ring,
X₁ to X₃, Y₁ to Y₂, Ar₁ to Ar₂, R₁ to R₄, o, p, q and r are the same as defined in claim 29.

35. The organic electroluminescent device of claim 29, wherein the compound is included as at least one of a light emitting layer, a light emitting auxiliary layer, an electron transport layer, and an auxiliary electron transport layer.
